(19) United States
(12) Patent Application Publication
(10) Pub. No.: US 2021/0079112 A1
(43) Pub. Date: Mar. 18, 2021

(54) COMBINATION OF NEAR INFRARED PHOTOIMMUNOTHERAPY TARGETING CANCER CELLS AND HOST-IMMUNE ACTIVATION

(71) Applicant: The United States of America, as represented by the Secretary, Department of Health and Human Ser, Bethesda, MD (US)

(72) Inventors: Hisataka Kobayashi, Laurel, MD (US); Peter Choyke, Rockville, MD (US)

(73) Assignee: The United States of America, as represented by the Secretary, Department of Health and Human Ser, Bethesda, MD (US)

(21) Appl. No.: 17/040,426

(22) PCT Filed: Apr. 9, 2019
(86) PCT No.: PCT/US2019/026488

§ 371 Date: Sep. 22, 2020

Related U.S. Application Data
(60) Provisional application No. 62/655,612, filed on Apr. 10, 2018.

(51) Int. Cl. C07K 16/30; A61P 35/00; C07K 16/28; C07K 16/32; A61N 5/00

(52) U.S. Cl. CPC C07K 16/30; A61P 35/00; C07K 16/2863; C07K 16/32; A61N 5/00; C07K 16/2887; C07K 16/2866; C07K 16/2803; C07K 16/2896; C07K 16/2884

(57) ABSTRACT

Provided herein are methods of treating a subject with cancer with a combination of antibody-IR700 molecules and immunomodulators. In particular examples, the methods include administering to a subject with cancer a therapeutically effective amount of one or more antibody-IR700 molecules, where the antibody specifically binds to a cancer cell surface protein, such as a tumor-specific antigen. The methods also include administering to the subject a therapeutically effective amount of one or more immunomodulators (such as an immune system activator or an inhibitor of immuno-suppressor cells), either simultaneously or substantially simultaneously with the antibody-IR700 molecules, or sequentially (for example, within about 0 to 24 hours). The subject or cancer cells in the subject (for example, a tumor or cancer cells in the blood) are then irradiated at a wavelength of 660 to 740 nm at a dose of at least 1 J/cm².

### CROSS-REFERENCE TO RELATED APPLICATION

This application claims priority to U.S. Provisional Application No. 62/655,612 filed Apr. 10, 2018, herein incorporated by reference in its entirety.

### ACKNOWLEDGMENT OF GOVERNMENT SUPPORT

This invention was made with Government support under project numbers Z01 ZIA BC 011513 and Z01 ZIA BC 010657 by the National Institutes of Health, National Cancer Institute. The Government has certain rights in the invention.

### FIELD

This disclosure relates to methods of using antibody-IR700 conjugates and in combination with one or more immunomodulators to kill cells, such as cancer cells, following irradiation with near infrared (NIR) light.

### BACKGROUND

Although there are several therapies for cancer, there remains a need for therapies that effectively kill the tumor cells while not harming non-cancerous cells.

In order to minimize the side effects of conventional cancer therapies, including surgery, radiation and chemotherapy, molecularly targeted cancer therapies have been developed. Among the existing targeted therapies, monoclonal antibodies (MAb) therapy have the longest history. Over 25 therapeutic MAbs have been approved by the Food and Drug Administration (FDA) (Waldmann, Nat Med 9:269-277, 2003; Reichert et al., Nat Biotechnol 23:1073-1078, 2005). Effective MAb therapy traditionally depends on three mechanisms: antibody-dependent cellular cytotoxicity (ADCC), complement-dependent cytotoxicity (CDC), and receptor blockade, and requires multiple high doses of the MAb. MAbs have also been used at lower doses as vectors to deliver therapies such as radionuclides (Goldenberg et al., J Clin Oncol 24, 823-834, 2006) or chemical or biological toxins (Pastan et al., Nat Rev Cancer 6:559-565, 2006). Ultimately, however, dose limiting toxicity relates to the biodistribution and catabolism of the antibody conjugates.

Conventional photodynamic therapy, which combines a photosensitizing agent with the physical energy of non-ionizing light to kill cells, has been less commonly employed for cancer therapy because the currently available non-targeted photosensitizers are also taken up in normal tissues, thus, causing side effects, although the excitation light itself is harmless in the near infrared (NIR) range. Cancer immunotherapy, which includes the use of immune modulatory antibodies, cancer vaccines, and cell-based therapies, has also become a strategy in the control of cancer (Chen and Mellman, Immunity 39:1-10, 2013; Childs and Carsten, Nat. Rev. Drug Discov. 14:487-498, 2015; June et al., Sci. Transl. Med. 7:280ps7, 2015; Melero et al., Nat. Rev. Cancer 15:457-472, 2015).

Near infrared photoimmunotherapy (NIR-PIT) is a cancer treatment that employs a targeted monoclonal antibody-photo-absorber conjugate (APC). Following antibody localization of the APC to a tumor cell surface antigen, NIR light is used to induce highly selective cytolysis. NIR-PIT induces rapid, necrotic cell death that yields innate immune ligands that activate dendritic cells (DCs), consistent with immunogenic cell death (ICD). A description of how NIR-PIT kills tumor cells is described in Sato et al. (ACS Cent. Sci. 4:1559-69, 2018). Briefly, following binding of the antibody-IR700 conjugate to its target, activation by NIR light causes physical changes in the shape of antibody-antigen complexes that induce physical stress within the cellular membrane, leading to increases in transmembrane water flow that eventually lead to cell bursting and necrotic cell death. Yet, NIR-PIT treatment of syngeneic tumors in wild-type mice has mostly failed to induce durable regression of established tumors.

### SUMMARY OF THE DISCLOSURE

Currently available cancer therapy aims either at directly targeting cancer cells or activating host immune system. No currently available cancer therapy achieves both killing cancer cells and activating host immune system against cancer cells. Additionally, no current cancer immunotherapies successfully produce long-time effective memory T-cells needed for complete treatment of cancer without concern about recurrence—a so-called “vaccine” effect. The methods disclosed herein can effectively produce long time acting memory T cells that significantly reduce or even prevent local or systemic recurrence of cancer.

Provided herein are methods of treating a subject with cancer with a combination of antibody-IR700 molecules and NIR-photoimmunotherapy (PIT) with immunomodulators. In particular examples, the methods include administering to a subject with cancer a therapeutically effective amount of one or more antibody-IR700 molecules, where the antibody specifically binds to a cancer cell surface molecule, such as a tumor-specific antigen. The methods also include administering to the subject a therapeutically effective amount of one or more immunomodulators (such as an immune system activator or an inhibitor of immuno-suppressor cells), either simultaneously or substantially simultaneously with the one or more antibody-IR700 molecules or sequentially (for example, within about 0 to 24 hours of one another). The subject or cancer cells in the subject (for example, a tumor, or cancer cells in the blood) are then irradiated at a wavelength of 660 to 740 nm, such as 660 to 710 nm (for example, 680 nm) at a dose of at least 1 J/cm² (such as at least 50 J/cm² or at least 100 J/cm²). In some examples, the method can further include selecting a subject with cancer having a tumor or cancer that expresses a cancer cell surface protein that can specifically bind to the antibody-IR700 molecule.

In some examples, the antibody-IR700 molecule includes an antibody that binds to one or more proteins on the cancer cell surface (such as a receptor), wherein the protein on the cancer cell surface is not significantly found on non-cancer cells (such as normal healthy cells) and thus the antibody will not significantly bind to the non-cancer cells. In one example the cancer cell surface protein is a tumor-specific protein, such as CD44, HER1, HER2, or PSMA. Additional exemplary tumor-specific proteins and antibodies are provided herein (including in Table 1, below).

In particular embodiments, the immunomodulators include one or more immune system activators and/or inhibitors of immuno-suppressor cells, such as an antagonistic PD-1 antibody, antagonistic PD-L1 antibody, or CD25 antibody-IR700 molecule. In some examples, the inhibitor of immuno-suppressor cells inhibits activity and/or kills regulatory T (Treg) cells. In other examples, the immune system activator includes one or more interleukins (such as IL-2 and/or IL-15). The immunomodulator may, in some examples, increase production of memory T cells specific for one or more proteins expressed by the cancer cells.

Also provided are methods of producing memory T cells specific for a target cell. In particular examples, the methods include administering to a subject a therapeutically effective amount of one or more antibody-IR700 molecules, where the antibody specifically binds to a cell surface molecule (such as a tumor-specific protein) on the target cell. The methods also include administering to the subject a therapeutically effective amount of one or more immunomodulators (such as an immune system activator or an inhibitor of immuno-suppressor cells), either simultaneously or substantially simultaneously with the antibody-IR700 molecules or sequentially (for example, within about 0 to 24 hours). The subject or target cells in the subject are then irradiated at a wavelength of 660 to 740 nm, such as 660 to 710 nm (for example, 680 nm) at a dose of at least 1 J/cm² (such as at least 50 J/cm² or at least 100 J/cm²), thereby producing memory T cells.

The foregoing and other features of the disclosure will become more apparent from the following detailed description of several embodiments, which proceeds with reference to the accompanying figures.

### BRIEF DESCRIPTION OF THE FIGURES

FIGS. 1A-1E are a series of panels showing in vitro effects of NIR-PIT with anti-CD44-IR700 on MC38-luc cells. FIG. 1A shows expression of CD44 in MC38-luc cells by FACS. FIG. 1B is a digital image showing differential interference contrast (DIC) and fluorescence microscopy images of control and anti-CD44-IR700 treated MC38-luc cells. Necrotic cell death was observed upon excitation with NIR light in treated cells. FIG. 1C is a digital image of bioluminescence imaging (BLI) of a 10-cm dish showing NIR light dose-dependent luciferase activity in MC38-luc cells. FIG. 1D is a graph showing luciferase activity in MC38-luc cells treated with NIR and with or without 10 μg/ml CD44-IR700. FIG. 1E is a graph showing percentage of cell death in MC38-luc cells treated with NIR with or without 10 μg/ml CD44-IR700, measured with dead cell count using propidium iodide (PI) staining. *, P<0.05 vs. untreated control; **, P<0.01 vs untreated control by Student t test.

FIGS. 1F and 1G are graphs showing percentage of cell death in (F) LLC cells or (G) MOC1 cells treated with NIR with or without 10 μg/ml CD44-IR700, measured with dead cell count using propidium iodide (PI) staining. *, P<0.05 vs. untreated control; **, P<0.01 vs untreated control by Student t test.

FIGS. 2A-2C Baseline CD44 expression within MOC1, LLC, and MC38-luc tumor compartments. (A) size matched MOC1 (day 24), LLC (day 10) and MC38-luc (day 10) tumors were harvested digested into a single cell suspension, and assessed for CD44 expression on individual cell types via flow cytometry (n=3/group). Representative dot plot and gating strategy of a tumor digest shown. Cell surface phenotype of each cell type shown above bar graphs.**p<0.01, ***p<0.001, t test with ANOVA. (B) In vivo CD44-IR700 fluorescence real-time imaging of tumor bearing mice. Images were obtained of MOC1 (day 18), LLC (day 4) and MC38-luc (day 4) tumors 24 hours after i.v. injection of CD44-IR700. The fluorescence intensity of CD44IR-700 was higher in MC38 tumor compared with the other two tumors. (C) Quantitative analysis of IR700 intensities in MOC1, LLC and MC38-luc tumors. The fluorescence intensities were significantly higher in MC38-luc tumors compared with other tumors (n≥10, ***p<0.001 vs MOC1 and LLC tumor, Tukey's test with ANOVA).

FIGS. 3A-3G are a series of panels showing in vivo effect of a combination therapy of cancer targeting PIT (anti-CD44-IR700) and a checkpoint inhibitor (anti-PD1) for MC38-luc tumor in a unilateral tumor model. (A) treatment scheme for unilateral tumor/NIR-PIT and fluorescence and bioluminescence imaging at the indicated timepoints; (B) In vivo IR700 fluorescence real-time imaging of tumor-bearing mice in response to NIR-PIT; (C) In vivo BLI of tumor bearing mice in response to NIR-PIT. Mice in the PD-1 mAb group also received CD44-IR700 but were not treated with NIR. (D) Quantification of luciferase activity in four treatment groups (n 10, **p<0.01 vs control, Tukey's t test with ANOVA; ^{#}p<0.05 vs PD-1 mAb and NIR-PIT groups, Tukey's t test with ANOVA). (E) Resected tumors (Day 10) were stained with H&E and assessed for necrosis and leukocyte infiltration. White scale bars=100 μm. Black scale bars=20 μm. (F) Tumor growth curves (n≥10, **p<0.01 vs control, Tukey's t test with ANOVA; ^{##}p<0.01 vs PD-1 mAb and NIR-PIT groups, Tukey's t test with ANOVA) and (G) Kaplan-Meier survival analysis following NIR-PIT treatment with and without PD-1 mAb (**p<0.01 vs control, Log rank test; np<0.01 vs PD-1 mAb and NIR-PIT groups, Log rank test).

FIGS. 4A-4D show the in vivo effect of NIR-PIT and PD-1 mAb in mice bearing a unilateral LLC tumor. (A) NIR-PIT regimen. Bioluminescence and fluorescence images were obtained at each time point as indicated. (B) In vivo IR700 fluorescence real-time imaging of tumor-bearing mice in response to NIR-PIT alone or in combination with PD-1 mAb. Mice in the PD-1 mAb group also received CD44-IR700 but were not treated with NIR. (C) LLC tumor growth curves following NIR-PIT treatment with and without PD-1 mAb (n≥10, **p<0.01 vs control, ##p<0.01 vs PD-1 mAb and NIR-PIT groups, Tukey's t test with ANOVA). (D) Kaplan-Meier survival analysis (n≥10, *p<0.05, **p<0.01 vs control, ##p<0.01 vs PD-1 mAb and NIR-PIT groups, Log rank test).

FIGS. 5A-5D show the in vivo effect of NIR-PIT and PD-1 mAb in mice bearing a unilateral MOC1 tumor. (A) NIR-PIT regimen. Bioluminescence and fluorescence images were obtained at each time point as indicated. (B) In vivo IR700 fluorescence real-time imaging of tumor-bearing mice in response to NIR-PIT alone or in combination with PD-1 mAb. Mice in the PD-1 mAb group also received CD44-IR700 but were not treated with NIR. (C) MOC1 tumor growth curves following NIR-PIT treatment with and without PD-1 (n≥10, **p<0.01 vs control, Tukey's test with ANOVA). (D) Kaplan-Meier survival analysis (n≥10, *p<0.05, **p<0.01 vs control, Log rank test).

FIGS. 6A-6F. Immune correlative and functional effects of NIR-PIT and PD-1 mAb in mice bearing a unilateral MC38-luc tumor. (A) MC38-luc tumors (day 10, n=5/group) treated with NIR-PIT with and without PD-1 mAb and controls were harvested, digested into single-cell suspensions, and analyzed for tumor infiltrating lymphocytes (TIL) infiltration via flow cytometry. Presented as absolute number of infiltrating cells per 1.5×10⁴ live cells analyzed. PD-1 expression shown as inset (MFI, mean fluorescence intensity). *p<0.05, **p<0.01, ***p<0.001, t test with ANOVA. (B) Multiplex immunofluorescence was used to validate flow cytometric data. Representative 400×images shown. Quantification of infiltrating TIL from 5 high power fields (HPF) per tumor, n=3/group. **p<0.01, ***p<0.001, t test with ANOVA. (C) TIL were extracted from tumors treated as above (n=5/group) via an IL-2 gradient, enriched via negative magnetic selection, and stimulated with irradiated splenocytes pulsed with peptides representing known MHC class I-restricted epitopes from selected tumor-associated antigens. IFNγ levels determined by ELISA from supernatants collected 24 hours after stimulation. Supernatants from splenocytes (APC) alone, TIL (T) alone, and a MHC-class I-restricted epitope from ovalbumin (OVA, SIINFEKL) used as controls. *p<0.05, **p<0.01, ***p<0.001, t test with ANOVA. (D) Flow cytometric analysis of tumor infiltrating dendritic cells (DC) and macrophages, with quantification of macrophage polarization based on MHC class II expression. **p<0.01, ***p<0.001, t test with ANOVA. (E) Flow cytometric analysis of tumor infiltrating neutrophilic myeloid cells (PMN-myeloid) and regulatory T-cells (T_{regs}). *p<0.05, **p<0.01, t test with ANOVA. (F) Flow cytometric analysis of PD-L1 expression on CD45.2⁻CD31⁻PDGFR⁻ tumor cells and CD45.2⁺CD31⁻ immune cells. **p<0.01 compared to control, t test with ANOVA. N=5/group.

FIGS. 7A-7E Immune correlative and functional effects of NIR-PIT and PD-1 mAb in mice bearing a unilateral LLC tumor. (A) LLC tumors (day 10, n=5/group) treated with NIR-PIT with and without systemic PD-1 mAb and controls were harvested, digested into single-cell suspensions, and analyzed for tumor infiltrating lymphocytes (TIL) infiltration via flow cytometry. Presented as absolute number of infiltrating cells per 1.5×10⁴ live cells analyzed. PD-1 expression shown as inset (MFI, mean fluorescence intensity). *p<0.05, **p<0.01, ***p<0.001, t test with ANOVA. (B) TIL were extracted from tumors treated as above (n=5/group) via an IL-2 gradient, enriched via negative magnetic selection, and stimulated with irradiated splenocytes pulsed with peptides representing known MHC class I-restricted epitopes from selected tumor-associated antigens. IFNγ levels determined by ELISA from supernatants collected 24 hours after stimulation. Supernatants from splenocytes (APC) alone, TIL (T) alone, and a MHC-class I-restricted epitope from ovalbumin (OVA, SIINFEKL) used as controls. *p<0.05, **p<0.01, t test with ANOVA. (C) Flow cytometric analysis of tumor infiltrating dendritic cells (DC) and macrophages, with quantification of macrophage polarization based on MHC class II expression. **p<0.01, ***p<0.001, t test with ANOVA. (D) Flow cytometric analysis of tumor infiltrating granulocytic myeloid derived suppressor cells PMN-myeloid and Tregs. **p<0.01, ***p<0.001, t test with ANOVA. (E) Flow cytometric analysis of PD-L1 expression on CD45.2-CD31-PDGFR-tumor cells and CD45.2⁺CD31-immune cells. N=5/group. *p<0.05, **p<0.01, ***p<0.001, t test with ANOVA.

FIGS. 8A-8E Immune correlative and functional effects of NIR-PIT and PD-1 mAb in MOC1 tumor-bearing mice. (A) MOC1 tumors (day 10, n=5/group) treated with NIR-PIT with and without systemic PD-1 mAb and controls were harvested, digested into single-cell suspensions, and analyzed for tumor infiltrating lymphocytes (TIL) infiltration via flow cytometry. Presented as absolute number of infiltrating cells per 1.5×104 live cells analyzed. PD-1 expression shown as inset (MFI, mean fluorescence intensity). *p<0.05, **p<0.01, t test with ANOVA. (B) TIL were extracted from tumors treated as above (n=5/group) via an IL-2 gradient, enriched via negative magnetic selection, and stimulated with irradiated splenocytes pulsed with peptides representing known MHC class I-restricted epitopes from selected tumor-associated antigens. IFNγ levels determined by ELISA from supernatants collected 24 hours after stimulation. Supernatants from splenocytes (APC) alone, TIL (T) alone, and a MHC-class I-restricted epitope from ovalbumin (OVA, SIINFEKL) used as controls. **p<0.01, t test with ANOVA. (C) Flow cytometric analysis of tumor infiltrating dendritic cells (DC) and macrophages, with quantification of macrophage polarization based on MHC class II expression. *p<0.05, **p<0.01, t test with ANOVA. (D) Flow cytometric analysis of tumor infiltrating PMN-myeloid and Tregs. (E) Flow cytometric analysis of PD-L1 expression on CD45.2-CD31-PDGFR-tumor cells and CD45.2⁺CD31-immune cells. N=5/group.

FIG. 9 Relative tumor associated antigen gene expression. MC38-luc, LLC and MOC1 cells were processed and assessed for gene expression of p15E, Birb5, Twist1 and Trp53by qRT-PCR using custom primers designed to flank the region encoding the MHC class I-restricted epitope (*p<0.05, **p<0.01, ***p<0.001, t test with ANOVA.). Two-dimensional plot of relative antigen expression level vs baseline antigen-specific IFNγ responses in TIL for each model shown on bottom.

FIGS. 10A-10H In vivo effect of NIR-PIT and PD-1 mAb in mice bearing bilateral MC38-luc tumors. (A) NIR-PIT regimen. Bioluminescence and fluorescence images were obtained at each time point as indicated. (B) NIR light was administered to the right-sided tumor only in mice bearing bilateral lower flank tumors. The untreated left-sided tumor was shielded from NIR light. (C) In vivo IR700 fluorescence real-time imaging of tumor-bearing mice in response to NIR-PIT to the right sided tumor only. (D) In vivo BLI of tumor bearing mice in response to combination NIR-PIT and PD-1 mAb. (E) Quantification of luciferase activity from each tumor, in controls and mice treated with combination NIR-PIT and PD-1 mAb (n=10, **p<0.01, Tukey's test with ANOVA). (F) Resected tumors (Day 10) were stained with H&E and assessed for necrosis and leukocyte infiltration. White scale bars=100 μm. Black scale bars=20 μm. (G) Growth curves of right- and left-sided tumors from controls and mice treated with combination NIR-PIT and PD-1 mAb. (H) Kaplan-Meier survival analysis from controls and mice treated with combination NIR-PIT and PD-1 mAb (n=10, **p<0.01, Tukey's test with ANOVA for growth curves; **p<0.01, Log-rank test for survival).

FIGS. 11A-11E. Immune correlative and functional effects of NIR-PIT and PD-1 mAb in mice bearing a bilateral MC38-luc tumors. (A) Bilateral MC38-luc tumors (day 10, n=5/group) treated with PD-1 mAb with or without NIR-PIT and bilateral control tumors were harvested, digested into single-cell suspensions, and analyzed for tumor infiltrating lymphocytes (TIL) infiltration via flow cytometry. Presented as absolute number of infiltrating cells per 1.5×10⁴ live cells analyzed. PD-1 expression shown as inset (MFI, mean fluorescence intensity). *p<0.05, ***p<0.001, t test with ANOVA. (B) TIL were extracted from tumors treated as above (n=5/group) via an IL-2 gradient, enriched via negative magnetic selection, and stimulated with irradiated splenocytes pulsed with peptides representing known MHC class I-restricted epitopes from selected tumor-associated antigens. IFNγ levels determined by ELISA from supernatants collected 24 hours after stimulation. Supernatants from splenocytes (APC) alone, TIL (T) alone, and a MHC-class I-restricted epitope from ovalbumin (OVA, SIINFEKL) used as controls. *p<0.05, ***p<0.001, t test with ANOVA. (C) Flow cytometric analysis of tumor infiltrating dendritic cells (DC) and macrophages, with quantification of macrophage polarization based on MHC class II expression. **p<0.01, ***p<0.001, t test with ANOVA. (D) Flow cytometric analysis of tumor infiltrating PMN-myeloid and T_{reg}s. *p<0.05, **p<0.01, t test with ANOVA. (E) Flow cytometric analysis of PD-L1 expression on CD45.2⁻CD31⁻PDGFR⁻ tumor cells. N=5/group.

FIGS. 12A-12H. In vivo effect of NIR-PIT and PD-1 mAb in mice bearing multiple MC38-luc tumors. (A) NIR-PIT regimen. Bioluminescence and fluorescence images were obtained at each time point as indicated. (B) NIR light was administered to the caudal right-sided tumor only in mice bearing four tumors. All other tumors were shielded from NIR light. (C) In vivo IR700 fluorescence real-time imaging of tumor-bearing mice in response to NIR-PIT treatment to the caudal right-sided tumor only. (D) In vivo BLI of tumor bearing mice in response to NIR-PIT treatment of the caudal right-sided tumor only. (E) Quantification of luciferase activity in all tumors from controls and mice treated with combination NIR-PIT and PD-1 mAb. Only the caudal right-sided tumor received NIR-PIT treatment (n=10, **p<0.01, Tukey's test with ANOVA). (F) Resected tumors (Day 10) were stained with H&E and assessed for necrosis and leukocyte infiltration. White scale bars=100 μm. Black scale bars=20 μm. (G) Growth curves from controls and treated and untreated tumors from mice receiving combination NIR-PIT and PD-1 mAb. (H) Kaplan-Meier survival analysis (n=10, **p<0.01, Tukey's test with ANOVA for growth curves; **p<0.01, Log-rank test for survival).

FIGS. 13A-13C. Resistance to re-challenge with MC38-luc cells following complete tumor rejection with combination NIR-PIT and PD-1 mAb treatment. (A) The regimen of tumor re-challenge in mice that completely rejected (CR) tumors with combination treatment. Tumor was inoculated on the contralateral side 30 days after first inoculation. Mice receiving re-inoculation of MC38-luc cells. (B) Growth curves of control and CR mice challenged with MC38-luc cells in the contralateral flank. (C) Kaplan-Meier survival analysis (n=9, ***p<0.001, by Tukey's test with ANOVA for growth curves, ***p<0.001, by Log-rank test for survival).

FIGS. 14A-14C. In vivo IR700 fluorescence imaging of MC38-luc, LL/2, and MOC1 tumor after injection of anti-CD25-mAb-IR700. (A) In vivo anti-CD25-mAb-IR700 fluorescence real-time imaging of tumor-bearing mice. In MC38-luc, LL/2, and MOC1 tumors, the tumor showed high fluorescence intensity after antibody-photo-absorber conjugate (APC) injection and the intensity gradually increased up to 24 hours after injection, stabilized and then decreased after 48 hours. (B) Quantitative analysis of mean fluorescence intensity (MFI) in MC38-luc, LL/2, and MOC1 tumors (n=5 in each group). The MFI of IR700 in MC38-luc, LL/2, and MOC1 tumors shows high uptake within 24 hours after APC injection whereupon it decreases after 48 hours. The overall MFI over time was significantly higher in MC38-luc tumors compared with MOC1 tumors at all time points (*p<0.05, MC38-luc vs. MOC1 tumors, Tukey-Kramer test), and the MFI at 24 and 48 hours was significantly higher in LL/2 tumors compared with MOC1 tumors (**p<0.05, LL/2 vs. MOC1 tumors, Tukey-Kramer test). (C) Quantitative analysis of target-to-background ratio (TBR) in MC38-luc, LL/2, and MOC1 tumors (n=5 in each group). TBR gradually increased up to 24 hours after APC injection, followed by decreased TBR after 48 hours. The TBR at 24 hours after was significantly higher in MC38-luc and LL/2 tumors compared with MOC1 tumors (*p<0.05, MC38-luc vs. MOC1 tumors, Tukey-Kramer test), and the TBR at 48 hours after was higher in LL/2 tumors compared with MOC1 tumors (**p<0.05, LL/2 vs. MOC1 tumors, Tukey-Kramer test).

FIGS. 15A-15F. In vivo effect of CD25- and/or CD44-targeted NIR-PIT for MC38-luc tumor model. (A) NIR-PIT regimen. Bioluminescence and fluorescence images were obtained at each time point as indicated. (B) In vivo IR700 fluorescence real-time imaging of tumor bearing mice in response to NIR-PIT. The tumor treated by NIR-PIT showed decreased IR700 fluorescence intensity immediately after NIR-PIT. (C) In vivo bioluminescence imaging of tumor bearing mice in response to NIR-PIT. Before NIR-PIT, tumors were approximately the same size and exhibited similar bioluminescence. The tumor treated by NIR-PIT showed decreased luciferase activity after NIR-PIT, whereupon it either gradually increased (regrowth) or disappeared (cure). (D) Quantitative analysis of luciferase activity before and after NIR-PIT in tumor bearing mice. Luciferase activity in all NIR-PIT treated groups showed significant decreases 2, 3, 4, 5, 6 and 7 days after NIR-PIT compared to the control group (n=13-14 mice in each group, *p<0.05 vs. the other groups, Tukey-Kramer test). Luciferase activity in combined CD25- and CD44-targeted NIR-PIT showed significant decrease 7 days after NIR-PIT compared to CD44-targeted NIR-PIT alone (n=13-14 mice in each group, **p<0.05 vs. combined NIR-PIT group, Tukey-Kramer test). (E) Tumor growth in all NIR-PIT treated groups was significantly inhibited 2, 5, 7 and 10 days after NIR-PIT compared to the control group (n=13-14 mice in each group, *p<0.05 vs. the other groups, Tukey-Kramer test). Combined CD25- and CD44-targeted NIR-PIT showed significant tumor reduction 7 and 10 days after NIR-PIT compared to CD44-targeted NIR-PIT alone (n=13-14 mice in each group, **p<0.05 vs. combined NIR-PIT group, Tukey-Kramer test). (F) Significantly prolonged survival was observed in all NIR-PIT treated groups compared to the control group (n=13-14 mice in each group, **p<0.01, Log-rank test). Combined CD25- and CD44-targeted NIR-PIT showed significantly prolonged survival compared to CD25-targeted NIR-PIT alone and CD44-targeted NIR-PIT alone (n=13-14 mice in each group, *p<0.05, **p<0.01, Log-rank test).

FIGS. 16A-16D. In vivo effect of CD25- and/or CD44-targeted NIR-PIT in LL/2 tumor model. (A) NIR-PIT regimen. IR700 fluorescence images were obtained at each time point as indicated. (B) In vivo IR700 fluorescence real-time imaging of tumor-bearing mice in response to NIR-PIT. The tumor treated by NIR-PIT showed decreased IR700 fluorescence intensity immediately after NIR-PIT. (C) Tumor growth in all NIR-PIT treated groups was significantly inhibited 5, 7, 10 and 12 days after NIR-PIT compared to the control group (n=9-10 mice in each group, *p<0.05 vs. the other groups, Tukey-Kramer test). Among all NIR-PIT treated groups, combined CD25- and CD44-targeted NIR-PIT showed significant tumor reduction 17 days after NIR-PIT compared with CD44-targeted NIR-PIT alone (n=9 mice in each group, **p<0.05 vs. combined NIR-PIT group, Tukey-Kramer test). (D) Significantly prolonged survival was observed in all NIR-PIT treated groups compared to the control group (n=9-10 mice in each group, **p<0.01, Log-rank test). Combined CD25- and CD44-targeted NIR-PIT showed significantly prolonged survival compared with CD25-targeted NIR-PIT alone and CD44-targeted NIR-PIT alone (n=9 mice in each group, *p<0.05, **p<0.01, Log-rank test).

FIGS. 17A-17D. In vivo effect of CD25- and/or CD44-targeted NIR-PIT in the MOC1 tumor model. (A) NIR-PIT regimen. IR700 fluorescence images were obtained at each time point as indicated. (B) In vivo IR700 fluorescence real-time imaging of tumor-bearing mice in response to NIR-PIT. The tumor treated by NIR-PIT showed decreased IR700 fluorescence intensity immediately after NIR-PIT. (C) Tumor growth in all NIR-PIT treated groups was significantly inhibited 4, 7, 10, 14, 17, 21, 24 and 28 days after NIR-PIT compared to the control group (n=9-10 mice in each group, *p<0.05 vs. the other groups, Tukey-Kramer test). Combined CD25- and CD44-targeted NIR-PIT showed significant tumor reduction 28 days after NIR-PIT compared with CD44-targeted NIR-PIT alone (n=9-10 mice in each group, **p<0.05 vs. combined NIR-PIT group, Tukey-Kramer test). (D) Significantly prolonged survival was observed in all NIR-PIT treated groups compared to the control group (n=9-10 mice in each group, **p<0.01, Log-rank test). Combined CD25- and CD44-targeted NIR-PIT showed significantly prolonged survival compared with CD44-targeted NIR-PIT alone (n=9-10 mice in each group, **p<0.01, Log-rank test).

FIG. 18. Scheme explaining the proposed mechanism of combined CD25- and CD44-targeted NIR-PIT-induced immunotherapy. Treg cells limit anti-tumor immunity through suppression of effector T cells and NK cells by inhibitory cytokines and cytolysis, as well as by metabolic disruption with IL-2 consumption, and by modulation of dendritic cell (DC) maturation or function. Combined CD25- and CD44-targeted NIR-PIT induces immunogenic cell death in CD44+ tumors and selectively depletes Treg cells highly expressing CD25. First, during the process of immunogenic cell death, exposure of surface calreticulin, heat shock protein (Hsp)70/90 and release of ATP and high mobility group box 1 (HMGB1) from dying tumor cells induce DC maturation. Second, Treg cell depletion induces activation and expansion of effector T cells and NK cells and simultaneously, differentiation into tumor-specific T cells. Taken together, this combined NIR-PIT results in effective tumor killing and promotion of long-lasting anti-tumor immunity.

### DETAILED DESCRIPTION OF SEVERAL EMBODIMENTS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

The sequences associated with all GenBank® Accession numbers referenced herein are incorporated by reference for the sequence available on Apr. 10, 2018.

In order to facilitate review of the various embodiments of the disclosure, the following explanations of specific terms are provided:

Administration: To provide or give a subject an agent, such as an antibody-IR700 molecule and/or an immunomodulator, by any effective route. Exemplary routes of administration include, but are not limited to, topical, systemic or local injection (such as subcutaneous, intramuscular, intradermal, intraperitoneal, intratumoral, and intravenous), oral, ocular, sublingual, rectal, transdermal, intranasal, vaginal, and inhalation routes.

Antibody: A polypeptide ligand comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies, such as those in an antibody-IR700 molecule, include intact immunoglobulins and the variants and portions of antibodies, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “V_{H}” refer to the variable region of an immunoglobulin heavy chain, including that of an Fv, scFv, dsFv or Fab. References to “V_{L}” or “V_{L}” refer to the variable region of an immunoglobulin light chain, including that of an Fv, scFv, dsFv or Fab.

A “monoclonal antibody” (mAb) is an antibody produced by a single clone of B lymphocytes or by a cell into which the light and heavy chain genes of a single antibody have been transfected. Monoclonal antibodies are produced for instance by making hybrid antibody-forming cells from a fusion of myeloma cells with immune spleen cells. Monoclonal antibodies include humanized monoclonal antibodies. In some examples, the antibody in an antibody-IR700 molecule is an mAb, such as a humanized mAb.

A “chimeric antibody” has framework residues from one species, such as human, and CDRs (which generally confer antigen binding) from another species, such as a murine antibody that specifically binds mesothelin.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, e.g., at least about 85-90%, such as about 95% or more identical.

Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

“Specifically binds” refers to the ability of individual antibodies to specifically immunoreact with an antigen, such as a tumor-specific antigen, relative to binding to unrelated proteins, such as non-tumor proteins, for example β-actin. For example, a HER2-specific binding agent binds substantially only the HER-2 protein in vitro or in vivo. As used herein, the term “tumor-specific binding agent” includes tumor-specific antibodies (and fragments thereof) and other agents that bind substantially only to a tumor-specific protein in that preparation.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody.”

In some examples, an antibody (such as one in an antibody-IR700 molecule) specifically binds to a target (such as a cell surface protein, such as a tumor specific protein) with a binding constant that is at least 10³ M⁻¹ greater, 10⁴M⁻¹ greater or 10⁵ M⁻¹ greater than a binding constant for other molecules in a sample or subject. In some examples, an antibody (e.g., mAb) or fragments thereof, has an equilibrium constant (Kd) of 1 nM or less. For example, an antibody binds to a target, such as tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Antibody-IR700 molecule or antibody-IR700 conjugate: A molecule that includes both an antibody, such as a tumor-specific antibody, conjugated to IR700. In some examples the antibody is a humanized antibody (such as a humanized mAb) that specifically binds to a surface protein on a cancer cell, such as a tumor-specific antigen.

Antigen (Ag): A compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific protein or peptide (such as one found on the surface of a cell, such as a cancer cell) or immunogenic fragment thereof.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, the cell killed by the disclosed methods is a cancer cell.

CD25 (IL-2 receptor alpha chain): (e.g., OMIM 147730) A type I transmembrane protein present on activated T cells, activated B cells, some thymocytes, myeloid precursors, and oligodendrocytes. CD25 has been used as a marker to identify CD4+FoxP3+ regulatory T cells in mice. CD25 is found on the surface of some cancer cells, including B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Exemplary CD25 sequences can be found on the GenBank® database (e.g., Accession Nos. CAA44297.1, NP_000408.1, and NP_001295171.1). Exemplary mAbs specific for CD25 are daclizumab and basiliximab, which can be attached to IR700, forming daclizumab-IR700 or basiliximab-IR700, which can be used in the disclosed methods to target CD25-expressing cancer cells, or used as an immunomodulator molecule (e.g., to reduce tumor-infiltrating Treg cells within the tumor).

CD44: (e.g., OMIM 107269) A cell-surface glycoprotein involved in cell-cell interactions, cell adhesion and migration. CD44 is found on the surface of some cancer cells, including cancer stem cells, head and neck cancer cells, breast cancer cells, and prostate cancer cells. Exemplary CD44 sequences can be found on the GenBank® database (e.g., Accession Nos. CAJ18532.1, ACI46596.1, and AAB20016.1). An exemplary mAb specific for CD44 is bivatuzumab, which can be attached to IR700, forming bivatuzumab-IR700, which can be used in the disclosed methods to target CD44-expressing cancer cells.

Contacting: Placement in direct physical association, including both a solid and liquid form. Contacting can occur in vitro, for example, with isolated cells, such as tumor cells, or in vivo by administering to a subject (such as a subject with a tumor, such as cancer).

Decrease: To reduce the quality, amount, or strength of something. In one example, a therapeutic composition that includes one or more antibody-IR700 molecules decreases the viability of cells to which the antibody-IR700 molecule specifically binds, following irradiation of the cells with NIR (for example at a wavelength of about 680 nm) at a dose of at least 1 J/cm², for example as compared to the response in the absence of the antibody-IR700 molecule. In some examples such a decrease is evidenced by the killing of the cells. In some examples, the decrease in the viability of cells is at least 20%, at least 50%, at least 75%, or even at least 90%, relative to the viability observed with a composition that does not include an antibody-IR700 molecule. In other examples, decreases are expressed as a fold change, such as a decrease in the cell viability by at least 2-fold, at least 3-fold, at least 4-fold, at least 5-fold, at least 8-fold, at least 10-fold, or even at least 15 or 20-fold, relative to the viability observed with a composition that does not include an antibody-IR700 molecule. Such decreases can be measured using the methods disclosed herein.

Immunomodulator: An immunomodulator is a substance that alters (for example, increases or decreases) one or more functions of the immune system. In some examples, an immunomodulator activates the immune system. In other examples, an immunomodulator inhibits activity of (or kills) immuno-suppressor cells.

IR700 (IRDye® 700DX): A dye having the following formula:

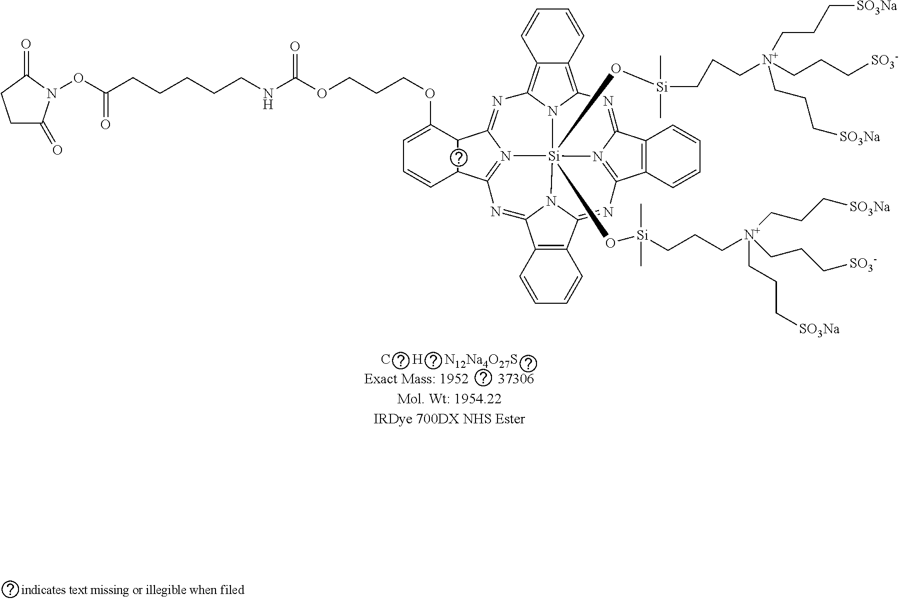

Commercially available from LI-COR (Lincoln, Nebr.). Amino-reactive IR700 is a relatively hydrophilic dye and can be covalently conjugated with an antiboidy using the NHS ester of IR700. IR700 also has more than 5-fold higher extinction coefficient (2.1×10⁵ M⁻¹cm⁻¹ at the absorption maximum of 689 nm), than conventional photosensitizers such as the hematoporphyrin derivative Photofrin® (1.2×10³M⁻¹ cm⁻¹ at 630 nm), meta-tetrahydroxyphenylchlorin; Foscan® (2.2×10⁴ M⁻¹cm⁻¹ at 652 nm), and mono-L-aspartylchlorin e6; NPe6/Laserphyrin® (4.0×10⁴ M⁻¹cm⁻¹ at 654 nm).

Pharmaceutical composition: A chemical compound or composition capable of inducing a desired therapeutic or prophylactic effect when properly administered to a subject. A pharmaceutical composition can include a therapeutic agent, such as one or more antibody-IR700 molecules and/or one or more immunomodulators. A therapeutic or pharmaceutical agent is one that alone or together with an additional compound induces the desired response (such as inducing a therapeutic or prophylactic effect when administered to a subject). In a particular example, a pharmaceutical composition includes a therapeutically effective amount of at least one antibody-IR700 molecule.

Pharmaceutically acceptable vehicles: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compounds, such as one or more antibody-IR700 molecules and/or one or more immunomodulators.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy (PIT): A molecularly targeted therapeutic that utilizes a target-specific photosensitizer based on a near infrared (NIR) phthalocyanine dye, IR700, conjugated to monoclonal antibodies (MAb) targeting cell surface protein. In one example the cell surface protein is one found specifically on cancer cells, and thus PIT can be used to kill such cells. Cell death occurs when the antibody-IR700 molecule binds to the cells and the cells are irradiated with NIR, while cells that do not express the cell surface protein recognized the antibody-IR700 molecule are not killed in significant numbers.

Programmed death 1 (PD-1): (e.g., OMIM 600244) A type 1 membrane protein on the surface of cells that has a role in regulating the immune system's response to the cells of the human body by down-regulating the immune system and promoting self-tolerance by suppressing T cell inflammatory activity. PD-1 binds to two ligands, PD-L1 and PD-L2. Exemplary PD-1 sequences can be found on the GenBank® database (e.g., Accession Nos. CAA48113.1, NP_005009.2, and NP_001076975.1).

Antibodies that antagonize PD-1 activity can be used as immunomodulators in the methods provided herein, for example in combination with a tumor-specific antigen Ab-IR700 molecule. Exemplary antagonistic mAbs specific for PD-1 include nivolumab, pembrolizumab, pidilizumab, cemiplimab, PDR001, AMP-224, and AMP-514.

Programmed death ligand 1 (PD-L1): (e.g., OMIM 605402) A type 1 membrane protein on the surface of cells that suppresses the adaptive arm of immune system during particular events such as pregnancy, tissue allografts, autoimmune disease and hepatitis. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM) motif. PD-L1 binds to PD-1, found on activated T cells, B cells, and myeloid cells, to modulate activation or inhibition. Exemplary PD-L1 sequences can be found on the GenBank® database (e.g., Accession Nos. ADK70950.1, NP_054862.1, and NP_001156884.1).

Antibodies that antagonize PD-L1 activity can be used can be used as immunomodulators in the methods provided herein, for example in combination with a tumor-specific antigen Ab-IR700 molecule. Exemplary antagonistic mAbs specific for PD-L1 include atezolizumab, avelumab, durvalumab, CK-301, and BMS-936559.

Subject or patient: A term that includes human and non-human mammals. In one example, the subject is a human or veterinary subject, such as a mouse, rat, dog, cat, or non-human primate. In some examples, the subject is a mammal (such as a human) who has cancer, or is being treated for cancer.

Therapeutically effective amount: An amount of a composition that alone, or together with an additional therapeutic agent(s) (such as a chemotherapeutic agent) sufficient to achieve a desired effect in a subject, or in a cell, being treated with the agent. The effective amount of the agent (such as an antibody-IR700 molecule, alone or in combination with an immunomodulator) can be dependent on several factors, including, but not limited to the subject or cells being treated, the particular therapeutic agent, and the manner of administration of the therapeutic composition. In one example, a therapeutically effective amount or concentration is one that is sufficient to prevent advancement (such as metastasis), delay progression, or to cause regression of a disease, or which is capable of reducing symptoms caused by the disease, such as cancer. In one example, a therapeutically effective amount or concentration is one that is sufficient to increase the survival time of a patient with a tumor.

In one example, a desired response is to reduce or inhibit one or more symptoms associated with cancer. The one or more symptoms do not have to be completely eliminated for the composition to be effective. For example, administration of a composition containing an antibody-IR700 molecule and a composition containing an immunomodulator (and/or a single composition containing both), in combination with irradiation can decrease the size of a tumor (such as the volume or weight of a tumor or metastasis of a tumor), for example by at least 20%, at least 50%, at least 80%, at least 90%, at least 95%, at least 98%, or even at least 100%, as compared to the tumor size in the absence of the treatment. In one particular example, a desired response is to kill a population of cells (such as cancer cells) by a desired amount, for example by killing at least 20%, at least 50%, at least 60%, at least 70%, at least 80%, at least 90%, at least 95%, at least 98%, or even at least 100% of the cells, as compared to the cell killing in the absence of the antibody-IR700 molecule, immunomodulator, and irradiation. In one particular example, a desired response is to increase the survival time of a patient with a tumor (or who has had a tumor recently removed) by a desired amount, for example increase survival by at least 20%, at least 50%, at least 60%, at least 70%, at least 80%, at least 90%, at least 95%, at least 98%, at least 100%, at least 200%, or at least 500%, as compared to the survival time in the absence of the antibody-IR700 molecule, immunomodulator, and irradiation. In some examples, a desired response is to increase an amount of memory T cells in a subject, for example increase by at least 20%, at least 50%, at least 60%, at least 70%, at least 80%, at least 90%, at least 95%, at least 98%, at least 100%, at least 200%, or at least 500%, as compared to an amount of memory T cells in the absence of the antibody-IR700 molecule, immunomodulator, and irradiation. In some examples, a desired response is to increase an amount of polyclonal antigen-specific TIC responses against MHC type I-restricted tumor specific antigens, in a subject, for example increase by at least 20%, at least 50%, at least 60%, at least 70%, at least 80%, at least 90%, at least 95%, at least 98%, at least 100%, at least 200%, or at least 500%, as compared to an amount of polyclonal antigen-specific TIC responses against MHC type I-restricted tumor specific antigens in the absence of the antibody-IR700 molecule, immunomodulator, and irradiation. In some examples, a desired response is to decrease an amount of Tregs (such as FOXP3⁺CD25⁺CD4⁺ Treg cells), in a targeted tumor, for example decrease by at least 20%, at least 50%, at least 60%, at least 70%, at least 80%, at least 90%, at least 95%, at least 98%, at least 100%, as compared to an amount of Tregs in the targeted tumor in the absence of the antibody-IR700 molecule, immunomodulator, and irradiation. In some examples, combinations of these effects are archived by the disclosed methods.

The effective amount of an agent that includes one or more of the disclosed antibody-IR700 molecules (alone or in combination with one or more immunomodulators) that is administered to a human or veterinary subject will vary depending upon a number of factors associated with that subject, for example the overall health of the subject. An effective amount of an agent can be determined by varying the dosage of the composition(s) and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount can be dependent on the treatment being applied, the subject being treated, the severity and type of the condition being treated, and the manner of administration.

In particular examples, a therapeutically effective dose of an antibody-IR700 molecule is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered iv. In another example, a therapeutically effective dose of an antibody-IR700 molecule is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered intratumorally or ip. In one example, a therapeutically effective dose is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in topical solution. However, one skilled in the art will recognize that higher or lower dosages also could be used, for example depending on the particular antibody-IR700 molecule. In particular examples, such daily dosages are administered in one or more divided doses (such as 2, 3, or 4 doses) or in a single formulation. The disclosed antibody-IR700 molecules can be administered alone, in the presence of a pharmaceutically acceptable carrier, in the presence of other therapeutic agents (such as other anti-neoplastic agents).

Generally a suitable dose of irradiation following administration of the one or more antibody-IR700 molecules and one or more immunomodulators is at least 1 J/cm² at a wavelength of 660-740 nm, for example, at least 10 J/cm² at a wavelength of 660-740 nm, at least 50 J/cm² at a wavelength of 660-740 nm, or at least 100 J/cm² at a wavelength of 660-740 nm, for example 1 to 500 J/cm² at a wavelength of 660-740 nm. In some examples the wavelength is 660-710 nm. In specific examples, a suitable dose of irradiation following administration of the antibody-IR700 molecule is at least 1.0 J/cm² at a wavelength of 680 nm for example, at least 10 J/cm² at a wavelength of 680 nm, at least 50 J/cm² at a wavelength of 680 nm, or at least 100 J/cm² at a wavelength of 680 nm, for example 1 to 500 J/cm² at a wavelength of 680 nm. In particular examples, multiple irradiations are performed (such as at least 2, at least 3, or at least 4 irradiations, such as 2, 3, 4, 5, 6, 7, 8, 9 or 10 separate administrations), following administration of the antibody-IR700 molecule and/or the immunomodulator.

Treating: A term when used to refer to the treatment of a cell or tissue with a therapeutic agent, includes contacting or incubating one or more agents (such as one or more antibody-IR700 molecules and one or more immunomodulators) with the cell or tissue and/or administering one or more agents to a subject, for example a subject with cancer. A treated cell is a cell that has been contacted with a desired composition in an amount and under conditions sufficient for the desired response. In one example, a treated cell is a cell that has been exposed to an antibody-IR700 molecule under conditions sufficient for the antibody to bind to a surface protein on the cell, contacted with an immunomodulator, and irradiated with NIR light, until sufficient cell killing is achieved. In other examples, a treated subject is a subject that has been administered one or more antibody-IR700 molecules under conditions sufficient for the antibody to bind to a surface protein on the cell, administered one or more immunomodulators, and irradiated with NIR light, until sufficient cell killing is achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Tumors include original (primary) tumors, recurrent tumors, and metastases (secondary) tumors. A tumor recurrence is the return of a tumor, at the same site as the original (primary) tumor, for example, after the tumor has been removed surgically, by drug or other treatment, or has otherwise disappeared. A metastasis is the spread of a tumor from one part of the body to another. Tumors formed from cells that have spread are called secondary tumors and contain cells that are like those in the original (primary) tumor. There can be a recurrence of either a primary tumor or a metastasis

Exemplary tumors, such as cancers, that can be treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, head and neck cancers (e.g., adenocarcinoma, squamous cell carcinoma, metastatic squamous, such as cancers caused by HPV or Epstein-Barr virus, such as HPV16; can include cancers of the mouth, tongue, nasopharynx, throat, hypopharynx, larynx, and trachea), stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The methods can also be used to treat liquid tumors (e.g., hematological malignancies), such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas.

Under conditions sufficient for: A phrase that is used to describe any environment that permits the desired activity. In one example, “under conditions sufficient for” includes administering an antibody-IR700 molecule to a subject sufficient to allow the antibody-IR700 molecule to bind to its targeted cell surface protein (such as a tumor-specific antigen). In particular examples, the desired activity is killing the cells to which the antibody-IR700 molecule is bound, following therapeutic irradiation of the cells.

Untreated: An untreated cell is a cell that has not been contacted with a therapeutic agent, such as an antibody-IR700 molecule, and immunomodulator, and/or irradiation. In an example, an untreated cell is a cell that receives the vehicle in which the therapeutic agent(s) was delivered. Similarly, an untreated subject is a subject that has not been administered a therapeutic agent, such as an antibody-IR700 molecule, and immunomodulator, and/or irradiation. In an example, an untreated subject is a subject that receives the vehicle in which the therapeutic agent(s) was delivered.

Disclosure of certain specific examples is not meant to exclude other embodiments. In addition, any treatments described herein are not necessarily exclusive of other treatment, but can be combined with other bioactive agents or treatment modalities.

### Overview of Technology

Near infrared photoimmunotherapy (NIR-PIT) is a highly-selective cancer treatment that induces necrotic/immunogenic cell death, utilizing a monoclonal antibody (mAb) conjugated to a photo-absorber IR700DX and NIR light. CD44 is associated with resistance to cancer treatment, but NIR-PIT employing an anti-CD44-mAb-IR700 conjugate is shown herein to inhibit cell growth and prolong survival in multiple tumor types. CD44 mAb-IR700 NIR-PIT targets a cancer antigen and initiates necrotic/immunogenic cell death, unlike apoptotic cell death that most other cancer therapies induce. Additional treatment with an immunomodulator (such as an immune checkpoint inhibitor, for example, an anti-PD1 antibody) synergized the anti-cancer effects of the anti-CD44-mAb-IR700 conjugate.

Furthermore, the methods successfully induced reduction of non-PIT treated distant tumors (e.g., metastases) and inhibited tumor recurrence upon later challenge with the same type of tumor cells. Thus the disclosed methods can also treat recurrences or metastases by eliciting host immunity (e.g., in some examples the methods reduce or eliminate tumor recurrence). PD-1 immune checkpoint blockade (ICB) reversed adaptive immune resistance following near infrared photoimmunotherapy to enhance a polyclonal T-cell response and induce rejection of established syngeneic tumors in both treated and distant untreated tumors. These polyclonal responses can also enhance formation of immunologic memory that suppress recurrence. This work is the first to definitively demonstrate development of de novo polyclonal T-cell responses (e.g., against multiple tumor associated antigens processed by dendritic cells) following tumor-targeting cytolytic therapy. In some examples, the disclosed methods cause selective depletion of Tregs, increase the number of memory T cells (such as tumor antigen specific T cells), increase dendritic cell tumor infiltration, or combinations thereof.

In some syngeneic mouse models, FOXP3⁺CD25⁺CD4⁺ Treg cells suppress host anti-tumor immunity mediated by inhibiting DC function through the CTLA4 axis or effector T or NK cell activation. Increased exposure to tumor antigens in the tumor micro environment (TME) in the presence of Treg cells may preferentially activate antigen-specific Treg cells rather than antigen-specific effector T cells. To overcome this, cancer and Treg cells were simultaneously targeted using combined CD44- and CD25-targeted NIR-PIT, which resulted in superior anti-tumor effects and prolonged survival compared to NIR-PIT using either target alone. In comparison, CD44-targeted NIR-PIT alone was markedly less effective in all three syngeneic tumor models investigated. Although Treg cells mediate tumor immune escape using various immunosuppressive mechanisms, CD25-targeted NIR-PIT can disable all of these mechanisms through selective Treg cell depletion. These results indicate that the disclosed methods result in superior in vivo therapeutic benefits (e.g., tumor growth inhibition and prolonged survival) over either cancer antigen-targeted NIR-PIT or elimination of immunosuppressive function alone. This combined NIR-PIT achieved some complete remissions whereas this was not the case with either type of NIR-PIT alone. Thus, the combined NIR-PIT method can result in long-term survival compared to conventional cancer antigen-targeted NIR-PIT, likely due to additive effects of direct tumor killing, induction of tumor immunogenicity through immunogenic cell death and effective activation of host anti-tumor immune cells derived from selective Treg cell depletion by CD25-targeted NIR-PIT. These three events, working together may elicit long term tumor responses in otherwise resistant tumors. Therefore, combined NIR-PIT with CD25- and CD44-targeted agents can eliminate both tumor cells and FOXP3⁺CD25⁺CD4⁺ Treg cells within targeted tumors. In addition, combined NIR-PIT simultaneously targeting cancer antigens and immunosuppressive cells in the TME may be even more highly efficient than one type of NIR PIT alone, which can be used to induce tumor vaccination.

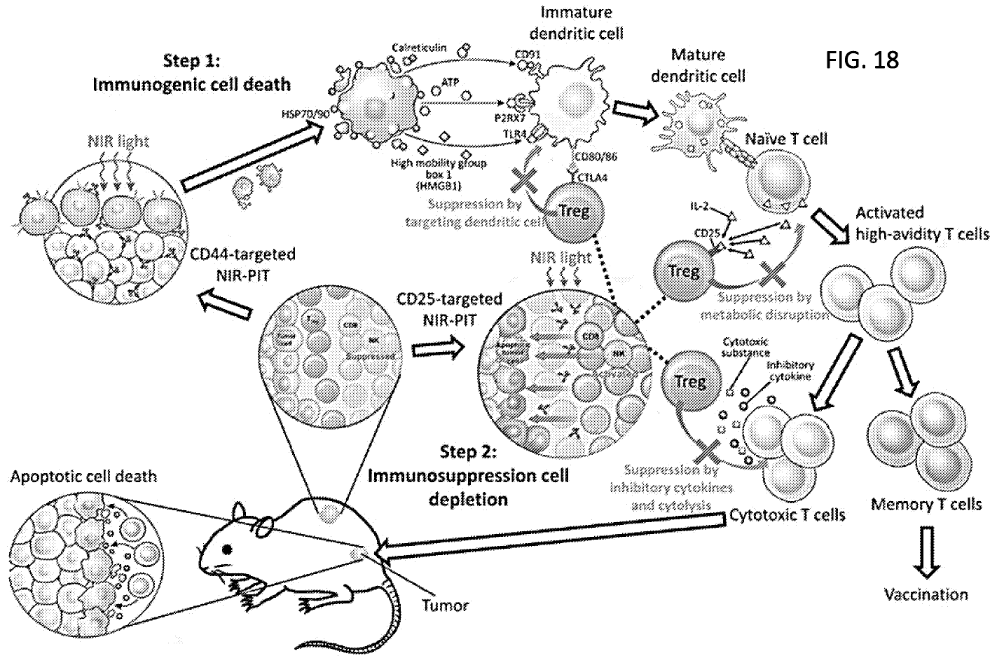

The presence of FOXP3⁺CD25⁺CD4⁺ Treg cells hinders development of tumor-specific high-avidity effector T cells although low-avidity effector T cells can function and expand. Treg-cell depletion enables activation and expansion of tumor-specific high-avidity T cells from naive T cell precursors, allowing their differentiation into high-avidity effector T cells capable of mediating potent anti-tumor immune responses. When this occurs, tumor vaccination is possible with combined CD25- and CD44-targeted NIR-PIT, due to activation of tumor-specific high-avidity effector or memory T cells which can lead to long-lasting anti-tumor immunity (FIG. 18). NIR-PIT can be repeatedly performed because it causes minimal damage to surrounding normal adjacent cells. Repeated dosing of antibody-photo-absorber conjugates (APCs) and NIR light can improve efficacy of NIR-PIT, increasing the frequency of successful vaccination in targeted tumors.

Based on these observations, provided herein are methods of treating a subject using NIR-PIT in combination with immunomodulation, which can locally kill cancer cells with minimal damage to surrounding cells or other cells not targeted by the antibody-IR700 molecule, and also provide effective anti-tumor host immune activation, resulting in highly effective treatment of various cancers using the subject's own immune system, both locally and even in distant metastases away from the treated site, with minimal side effects. In some examples, treatment of a single local site with the disclosed methods permits systemic host immunity against cancers, leading to rapid tumor regression at the treated site as well as untreated distant metastatic lesions, while inducing minimal side effects.

### Methods for Treating Cancer

The present disclosure provides methods for treating a subject with cancer, such as a subject with a tumor or a hematological malignancy. The methods include administering to the subject an antibody that is conjugated to the dye IR700 (referred to herein as an antibody-IR700 molecule), wherein the antibody specifically binds to a cancer (e.g., tumor) cell surface protein (also referred to herein as a tumor-specific antigen or protein). The subject is administered a therapeutically effective amount of one or more antibody-IR700 molecules (for example in the presence of a pharmaceutically acceptable carrier, such as a pharmaceutically and physiologically acceptable fluid), under conditions that permit the antibody to specifically bind to the cancer cell surface protein. For example, the antibody-IR700 molecule can be present in a pharmaceutically effective carrier, such as water, physiological saline, balanced salt solutions (such as PBS/EDTA), aqueous dextrose, sesame oil, glycerol, ethanol, combinations thereof, or the like, as a vehicle. The carrier and composition can be sterile, and the formulation suits the mode of administration. In a specific example, the antibody-IR700 molecule is CD44 antibody-IR700.

The methods also include administering to the subject a therapeutically effective amount of one or more immunomodulators, such as one or more immune system activators and/or one or more inhibitors of immuno-suppressor cells (for example in the presence of a pharmaceutically acceptable carrier, such as a pharmaceutically and physiologically acceptable fluid). In a specific example, the immunomodulatory agent is a PD-1 or PD-L1 antibody. In another specific example, the immunomodulatory agent is CD25 antibody-IR700. In some examples, the one or more immunomodulators are administered to the subject concurrently (for example, simultaneously or substantially simultaneously) with the one or more antibody-IR700 molecules that bind to the cancer cell surface protein, for example in the same composition, or if administered as separate compositions, within about 1 hour of one another (for example, within about 5 minutes, about 10 minutes, about 15 minutes, about 20 minutes, about 30 minutes, about 40 minutes, about 50 minutes, or about 60 minutes). In other examples, the one or more antibody-IR700 molecules that bind to the cancer cell surface protein and the one or more immunomodulators are administered to the subject sequentially (in either order), for example, separated by at least about 1 hour to one week (for example, separated by about 2 hours, about 12 hours, about 24 hours, about 48 hours, about 3 days, about 4 days, about 5 days, about 6 days, or about 7 days).

After administering the one or more antibody-IR700 molecules, the one or more antibody-IR700 molecules are allowed to accumulate in the targeted tumor. The cancer cells (or the subject having the cancer) are then irradiated under conditions that permit killing of the cells, for example irradiation at a wavelength of 660 to 710 nm at a dose of at least 1 J/cm². In one example, there is at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours) in between administering the antibody-IR700 molecules and the irradiation. In one example, the one or more antibody-IR700 molecules are administered (e.g., i.v.) and at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours, such as about 24 hours) later, the tumor (or the subject) is irradiated. The one or more immunomodulators may be administered before or after the one or more antibody-IR700 molecules and/or before or after the irradiation. In some examples, the one or more immunomodulators are administered before and after irradiation, for example, at least one dose of immunomodulators prior to irradiation and at least one dose of immunomodulators after irradiation (such as 24 hours before and one or more of 24, 48, 72, 96, or more hours after irradiation). In additional examples, a dose of immunomodulators may also be administered on the same day as at least one irradiation treatment.

In some examples, multiple doses of one or more of the antibody-IR700 molecule(s), immunomodulator(s), and irradiation with NIR are administered to the subject, such as at least 2, at least 3, at least 4, at least 5, at least 6, at least 7, at least 8, at least 9, or at least 10 separate doses (or administrations). In a specific example, the subject is administered at least one dose of the one or more of the antibody-IR700 molecule(s), at least two doses of the one or more immunomodulator(s), and at least two administrations of NIR irradiation.

The NIR excitation light wavelength allows penetration of at least several centimeters into tissues. For example, by using fiber-coupled laser diodes with diffuser tips, NIR light can be delivered within several centimeters of otherwise inaccessible tumors located deep with respect to the body surface. In addition to treating solid cancers, circulating tumor cells (including, but not limited to hematological malignancies) can be targeted since they can be excited when they traverse superficial vessels (for example using the NIR LED wearable devices described herein).

In one example, administering to the subject one or more antibody-IR700 molecules and one or more immunomodulators, in combination with irradiation, kills target cells (such as cancer cells) that express a cell surface protein (such as a tumor-specific antigen) that specifically binds to the antibody. For example, the disclosed methods in some examples kill at least 10%, for example at least 20%, at least 40%, at least 50%, at least 80%, at least 90%, at least 95%, at least 98%, or more of the treated target cells (such as cancer cells expressing the tumor-specific antigen) relative to the absence of treatment with of one or more antibody-IR700 molecules and administration of one or more immunomodulators, in combination with irradiation.

In one example, administration of one or more antibody-IR700 molecules and administration of one or more immunomodulators to a subject having a tumor, in combination with irradiation, selectively kills the cells that express a cell surface protein (such as a tumor-specific antigen) that can specifically bind to the antibody, thereby treating the tumor. By selective killing of tumor cells relative to normal cells is meant that the methods are capable of killing tumor cells more effectively than normal cells such as, for example, cells not expressing the cell surface protein (such as a tumor-specific antigen) that specifically binds to the antibody administered. For example, the disclosed methods in some examples decrease the size or volume of a tumor, slow the growth of a tumor, decrease or slow recurrence of the tumor, decrease or slow metastasis of the tumor (for example by reducing the number of metastases or decreasing the volume or size of a metastasis), or combinations thereof. For example, the disclosed methods in some examples reduce tumor size (such as weight or volume of a tumor) or number of tumors and/or reduce metastatic tumor size or number of metastatic tumors, such as by at least 10%, for example by at least 20%, at least 40%, at least 50%, at least 80%, at least 90%, at least 95%, at least 98%, or more, relative to the absence of administration of one or more antibody-IR700 molecules and administration of one or more immunomodulators, in combination with irradiation.

In one example, administration of one or more antibody-IR700 molecules and administration of one or more immunomodulators to a subject having a tumor, in combination with irradiation, decreases Tregs (such as FOXP3⁺CD25⁺CD4⁺ Treg cells). For example, the disclosed methods in some examples decrease the number of circulating Tregs by at least 10%, for example by at least 20%, at least 40%, at least 50%, at least 80%, at least 90%, at least 95%, at least 98%, or more, relative to the absence of administration of one or more antibody-IR700 molecules and administration of one or more immunomodulators, in combination with irradiation. In some examples, the disclosed methods decrease the number of Tregs in a tumor by at least 10%, for example by at least 20%, at least 40%, at least 50%, at least 80%, at least 90%, at least 95%, at least 98%, or more, relative to the absence of administration of one or more antibody-IR700 molecules and administration of one or more immunomodulators, in combination with irradiation.

In one example, administration of one or more antibody-IR700 molecules and administration of one or more immunomodulators to a subject having a tumor, in combination with irradiation, increases memory T cells. For example, the disclosed methods in some examples increase the number of circulating memory T cells by at least 10%, for example by at least 20%, at least 40%, at least 50%, at least 80%, at least 90%, at least 95%, at least 98%, at least 100%, at least 200%, at least 300%, at least 400%, at least 500%, or more, relative to the absence of administration of one or more antibody-IR700 molecules and administration of one or more immunomodulators, in combination with irradiation.

In some examples, the disclosed methods decrease one or more symptoms associated with a tumor, a recurrence, and/or a metastatic tumor. In one example, the disclosed methods slow the growth of a tumor, such as by at least 10%, for example by at least 20%, at least 40%, at least 50%, at least 80%, at least 90%, or more, relative to the absence of administration of the antibody-IR700 molecules and one or more immunomodulators, in combination with irradiation. In one example, the disclosed methods reduce or eliminates tumor recurrence, such as by at least 10%, for example by at least 20%, at least 40%, at least 50%, at least 80%, at least 90%, at least 95%, at least 98%, at least 99% or even 100%, relative to the absence of administration of the antibody-IR700 molecules and one or more immunomodulators, in combination with irradiation.

In some examples, the disclosed methods can increase a subject's (such as a subject with a tumor or who has had a tumor previously removed) survival time, for example relative to the absence of administration of one or more antibody-IR700 molecules and one or more immunomodulators and irradiation, such as an increase of at least 20%, at least 40%, at least 50%, at least 80%, at least 90%, or more. For example, the disclosed methods in some examples increase a subject's overall survival time and/or progression-free survival time (for example, lack of recurrence of the primary tumor or lack of metastasis) by at least 1 months, at least 2 months, at least 3 months, at least 6 months, at least 12 months, at least 18 months, at least 24 months, at least 36 months, at least 48 months, at least 60 months, or more, relative to average survival time in the absence of administration of an antibody-IR700 molecule, one or more immunomodulators, and irradiation.

In one example, administration of a composition containing an antibody-IR700 molecule and administration of one or more immunomodulators (concurrently or sequentially), in combination with NIR irradiation of a primary tumor can decrease the size and/or number of a distant non-irradiated tumors or tumor metastases (such as the volume of a distant tumor or metastasis, weight of a distant tumor or metastasis, number of distant tumors or metastases, or combinations thereof), for example by at least 20%, at least 50%, at least 80%, at least 90%, at least 95%, at least 98%, or even at least 100%, as compared to the volume/weight/number of distant tumors or metastases in the absence of the antibody-IR700 molecule, the immunomodulator, and NIR irradiation of the primary tumor.

In one example, the disclosed methods increase an amount of polyclonal antigen-specific TIC responses against MHC type I-restricted tumor specific antigens, in a subject, for example increase by at least 20%, at least 50%, at least 60%, at least 70%, at least 80%, at least 90%, at least 95%, at least 98%, at least 100%, at least 200%, or at least 500%, as compared to an amount of polyclonal antigen-specific TIC responses against MHC type I-restricted tumor specific antigens in the absence of the antibody-IR700 molecule, immunomodulator, and irradiation.

In one example, combinations of these effects are achieved with the disclosed methods.

The disclosed methods can be used to treat fixed tumors in the body as well as hematological malignancies and/or tumors in the circulation (e.g., leukemia cells, metastases, and/or circulating tumor cells). However, circulating cells, by their nature, cannot be exposed to light for very long. Thus, if the cell to be killed is one that is circulating throughout the body, the methods can be accomplished by using a device that can be worn, or that covers parts of the body. For example, such a device can be worn for extended time periods. Everyday wearable items (e.g., wristwatches, jewelry (such as a necklace or bracelet), blankets, clothing (e.g., underwear, socks, and shoe inserts) and other everyday wearable items) which incorporate NIR emitting light emitting diodes (LEDs) and a battery pack, can be used. Such devices produce light on the skin underlying the device over long periods leading to continual exposure of light to superficial vessels over prolonged periods. Circulating tumor cells are exposed to the light as they transit thru the area underlying the device. As an example, a wristwatch or bracelet version of this device can include a series of NIR LEDs with battery power pack to be worn for most of the day. After administration of the one or more antibody-IR700 molecules (e.g., intravenously), circulating cells bind the antibody-IR700 conjugate and become susceptible to killing by PIT. As these cells flow within the vessels adjacent to the LED present in the everyday wearable item (e.g., bracelet or wristwatch), they would be exposed to NIR light rendering them susceptible to cell killing. The dose of light may be adjustable according to diagnosis and cell type.

In some examples, the method further includes monitoring the therapy, such as killing of tumor cells. In such examples, the subject is administered the antibody-IR700 conjugate and immunomodulators and irradiated as described herein. However, a lower dose of the antibody-IR700 conjugate and NIR light can be used for monitoring (as cell killing may not be required, just monitoring of the therapy). In one example, the amount of antibody-IR700 conjugate administered for monitoring is at least 2-fold less (such as at least 3-, 4-, 5-, 6-, 7-, 8-, 9-, or 10-fold less than the therapeutic dose). In one example, the amount of antibody-IR700 conjugate administered for monitoring is at least 20% or at least 25% less than the therapeutic dose. In one example, the amount of NIR light used for monitoring is at least 1/1000 or at least 1/10,000 of the therapeutic dose. This permits detection of the cells being treated. For example, by using such methods, the size of the tumor and metastases can be monitored.

In some examples, the method is useful during surgery, such as endoscopic procedures. For example, after the antibody-IR700 conjugate and the immunomodulator are administered to the subject and the cells irradiated as described above, this not only results in cell killing, but permits a surgeon or other medical care provider to visualize the margins of a tumor, and help ensure that resection of the tumor (such as a tumor of the skin, breast, lung, colon, head and neck, or prostate) is complete and that the margins are clear. In some examples, a lower dose of the antibody-IR700 conjugate can be used for visualization, such as at least 2-fold less (such as at least 3-, 4-, 5-, 6-, 7-, 8-, 9-, or 10-fold less than the therapeutic dose).

The antibody-IR700 molecules and immunomodulators can be administered locally or systemically, for example to subjects having a tumor, such as a cancer, or who has had a tumor previously removed (for example via surgery). Although specific examples are provided, one skilled in the art will appreciate that alternative methods of administration of the disclosed antibody-IR700 molecules and immunomodulators can be used. Such methods may include for example, the use of catheters or implantable pumps to provide continuous infusion over a period of several hours to several days into the subject in need of treatment.

In one example, the antibody-IR700 molecules and/or immunomodulators are administered by parenteral means, including direct injection or infusion into a tumor (intratumorally). In some examples, the antibody-IR700 molecules and/or immunomodulators are administered to the tumor by applying the antibody-IR700 molecules and/or immunomodulators to the tumor, for example by local injection of antibody-IR700 molecules and/or immunomodulators, bathing the tumor in a solution containing the antibody-IR700 molecules and/or immunomodulators, or by pouring the antibody-IR700 molecules and/or immunomodulators onto the tumor.

In addition, or alternatively, the disclosed compositions can be administered systemically, for example intravenously, intramuscularly, subcutaneously, intradermally, intraperitoneally, subcutaneously, or orally, to a subject having a tumor (such as cancer). The one or more antibody-IR700 molecules and one or more immunomodulators may be administered by the same or different routes. In one example, the antibody-IR700 molecules may be administered intratumorally and the immunomodulators may be delivered systemically (for example, intravenously or intraperitoneally). In another example, the antibody-IR700 molecule and the immunomodulator are administered systemically (for example, intravenously or intraperitoneally). In one example, the antibody-IR700 molecule is administered intravenously, and the immunomodulator intraperitoneally. In one example, the antibody-IR700 molecule and the immunomodulator are administered intravenously.

The dosages of the antibody-IR700 molecules and immunomodulators to be administered to a subject are not subject to absolute limits, but will depend on the nature of the composition, its active ingredients and its potential unwanted side effects (e g, immune response against the antibody), the subject being treated and the type of condition being treated, and the manner of administration. Generally the dose will be a therapeutically effective amount, such as an amount sufficient to achieve a desired biological effect, for example an amount that is effective to decrease the size (e.g., volume and/or weight) of the tumor, or attenuate further growth of the tumor, or decrease undesired symptoms of the tumor.

For intravenous administration of antibody-IR700 molecules (including tumor-specific antibody-IR700 molecules and immunomodulator antibody-IR700 molecules), exemplary dosages for administration to a subject for a single treatment can range from 0.5 to 100 mg/60 kg of body weight, 1 to 100 mg/60 kg of body weight, 1 to 50 mg/60 kg of body weight, 1 to 20 mg/60 kg of body weight, for example about 1 or 2 mg/60 kg of body weight. In yet another example, a therapeutically effective amount of intraperitoneally or intratumorally administered antibody-IR700 molecules is 10 μg to 5000 μg of antibody-IR700 molecule per 1 kg of body weight, such as 10 μg/kg to 1000 μg/kg, 10 μg/kg to 500 μg/kg, or 100 μg/kg to 1000 μg/kg. In one example, the dose of antibody-IR700 molecule administered to a human patient is at least 50 mg, such as at least 100 mg, at least 300 mg, at least 500 mg, at least 750 mg, or even 1 g. Similar amounts of antibodies that are not conjugated to IR700 (such as immunomodulator antibodies, such as those specific for PD-1 or PD-L1) may also be used.

Treatments with disclosed antibody-IR700 molecules and immunomodulators can be completed in a single day, or may be done repeatedly on multiple days with the same or a different dosage. Repeated treatments may be done on the same day, on successive days, or every 1-3 days, every 3-7 days, every 1-2 weeks, every 2-4 weeks, every 1-2 months, or at even longer intervals. In some examples, the antibody-IR700 molecules and immunomodulators are administered on the same day. In other examples, the antibody-IR700 molecules and immunomodulators are administered on different days. In one non-limiting example, the one or more antibody-IR700 molecules and one or more immunomodulators are administered to the subject on the same day and repeated doses of the one or more immunomodulators (at the same or different dosing level) are administered to the subject (for example, 1, 2, 3, 4, 5, or more additional doses of the immunomodulator) on successive days, or every 1-3 days, every 3-7 days, every 1-2 weeks, every 2-4 weeks, every 1-2 months, or at even longer intervals. In some examples, the amount of the repeated doses of the immunomodulator is reduced compared to the initial dose (for example, reduced by 50% in some cases).

In additional embodiments, the methods also include administering to the subject one or more additional therapeutic agents. As described in International Patent Application Publication No. WO 2013/009475 (incorporated by reference herein in its entirety), there is about an 8 hour window following irradiation (for example irradiation at a wavelength of 660 to 710 nm at a dose of at least 10 J/cm², at least 20 J/cm², at least 30 J/cm², at least 40 J/cm², at least 50 J/cm², at least 70 J/cm², at least 80 J/cm² or at least 100 J/cm², such as at least 10 to 100 J/cm²), during which uptake of additional agents (e.g., nano-sized agents, such as those about at least 1 nm in diameter, at least 10 nm in diameter, at least 100 nm in diameter, or at least 200 nm in diameter, such as 1 to 500 nm in diameter) by the PIT-treated cells is enhanced. Thus, one or more additional therapeutic agents can further be administered to the subject contemporaneously or sequentially with the PIT. In one example, the additional therapeutic agents are administered after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation). Additional therapeutic agents that can be used are discussed below.

In additional embodiments, methods are provided that permit detection or monitoring of cell killing in real-time. Such methods are useful for example, to ensure sufficient amounts of antibody-IR700 molecules and/or immunomodulators, or sufficient amounts of irradiation, were delivered to the cell or tumor to promote cell killing. These methods permit detection of cell killing before morphological changes become evident. In one example, the methods include contacting a cell having a cell surface protein with a therapeutically effective amount of one or more antibody-IR700 molecules and one or more immunomodulators, wherein the antibody specifically binds to the cell surface protein (for example, administering the antibody-IR700 molecule(s) and immunomodulator(s) to a subject); irradiating the cell at a wavelength of 660 to 740 nm and at a dose of at least 20 J/cm²; and detecting the cell with fluorescence lifetime imaging about 0 to 48 hours after irradiating the cell (such as at least 1 hour, at least 2 hours, at least 4 hours, at least 6 hours, at least 12 hours, at least 18 hours, at least 24 hours, at least 36 hours, at least 48 hours, or at least 72 hours after irradiating the cell, for example 1 minute to 30 minutes, 10 minutes to 30 minutes, 10 minutes to 1 hour, 1 hour to 8 hours, 6 hours to 24 hours, or 6 hours to 48 hours after irradiating the cell), thereby detecting the cell killing in real-time. Shortening FLT serves as an indicator of acute membrane damage induced by PIT. Thus, the cell is irradiated under conditions sufficient to shorten IR700 FLT by at least 25%, such as at least 40%, at least 50%, at least 60% or at least 75%. In one example, the cell is irradiated at a wavelength of 660 nm to 740 nm (such as 680 nm to 700 nm) and at a dose of at least 20 J/cm² or at least 30 J/cm², such as at least 40 J/cm² or at least 50 J/cm² or at least 60 J/cm², such as 30 to 50 J/cm².

In some examples, methods of detecting cell killing in real time includes contacting the cell with one or more additional therapeutic agents, for example about 0 to 8 hours after irradiating the cell. The real-time imaging can occur before or after contacting the cell with one or more additional therapeutic agents. For example, if insufficient cell killing occurs after administration of the one or more antibody-IR700 molecules and one or more immunomodulators as determined by the real-timing imaging, then the cell can be contacted with one or more additional therapeutic agents. However, in some examples, the cell is contacted with the antibody-IR700 molecules and immunomodulators and additional therapeutic agents prior to detecting the cell killing in real-time.

### Exemplary Cells

The target cell can be a cell that is not desired or whose growth is not desired, such as a cancer cell (e.g., a tumor cell). The cells can be present in a mammal to be treated, such as a subject (for example, a human or veterinary subject) with cancer. Any target cell can be treated with the claimed methods. In one example, the target cell expresses a cell surface protein that is not substantially found on the surface of other normal (desired) cells, an antibody can be selected that specifically binds to such protein, and an antibody-IR700 molecule generated for that protein. In one example, the cell surface protein is a tumor-specific protein (e.g., antigen). In one non-limiting example, the cell surface protein is CD44.

In one example, the tumor cell is a cancer cell, such as a cell in a patient with cancer. Exemplary cells that can be killed with the disclosed methods include cells of the following tumors: a hematological malignancy such as a leukemia, including acute leukemia (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrdm's macroglobulinemia, heavy chain disease). In another example the cell is a solid tumor cell, such as cells from sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinomna, lung cancer, colorectal cancer, squamous cell carcinoma, a head and neck cancer (such as head and neck squamous cell carcinoma), basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS cancers (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

In a specific example, the cell is a lung cancer cell.

In a specific example, the cell is a breast cancer cell.

In a specific example, the cell is a colon cancer cell.

In a specific example, the cell is a head and neck cancer cell.

In a specific example, the cell is a prostate cancer cell.

### Exemplary Subjects

In some examples the disclosed methods are used to treat a subject who has cancer or a subject with a tumor, such as a tumor described herein. In some examples, the tumor has been previously treated, such as surgically or chemically removed, and the disclosed methods are used subsequently to kill any remaining undesired tumor cells that may remain in the patient and/or reduce recurrence or metastasis of the tumor.

The disclosed methods can be used to treat any mammalian subject (such as a human or veterinary subject, such as a dog or cat), such as a human, who has a tumor, such as a cancer, or has had such previously removed or treated. Subjects in need of the disclosed therapies can include human subjects having cancer, wherein the cancer cells express a tumor-specific protein on their surface that can specifically bind to the antibody-IR700 molecule. For example, the disclosed methods can be used as initial treatment for cancer either alone, or in combination with radiation or other chemotherapy. The disclosed methods can also be used in patients who have failed previous radiation or chemotherapy. Thus, in some examples, the subject is one who has received other therapies, but those other therapies have not provided a desired therapeutic response. The disclosed methods can also be used in patients with localized and/or metastatic cancer and/or a recurrence of a primary tumor.

In some examples the method includes selecting a subject that will benefit from the disclosed therapies, such as selecting a subject having a tumor that expresses a cell surface protein (such as a tumor-specific protein) that can specifically bind to an antibody-IR700 molecule. For example, if the subject is determined to have a breast cancer that expresses HER2, the subject can be selected to be treated with an anti-HER2-IR700 molecule, such as Trastuzumab-IR700 and one or more immunomodulators, and the subject is subsequently irradiated as described herein.

### Exemplary Cell Surface Proteins

In one example, the protein on the cell surface of the target cell to be killed is not present in significant amounts on other cells. For example, the cell surface protein can be a receptor that is only found on the target cell type.

In a specific example, the cell surface protein is a cancer- or tumor-specific protein (also known in the art as a tumor-specific antigen or tumor-associated antigen), such as members of the EGF receptor family (e.g., HER1, 2, 3, and 4) and cytokine receptors (e.g., CD20, CD25, IL-13R, CD5, CD52, etc.). Thus, in some examples, the cell surface protein is an antigen expressed on the cell membrane of tumor cells. Tumor-specific proteins are proteins that are unique to cancer cells or are much more abundant on them, as compared to other cells, such as normal cells. For example HER2 is primarily found in breast cancers, while HER1 is primarily found in adenocarcinomas, which can be found in many organs, such as the pancreas, breast, prostate and colon.

Exemplary tumor-specific proteins that can be found on a target cell (and to which an antibody specific for that protein can be used to formulate an antibody-IR700 molecule), include but are not limited to: any of the various MAGEs (Melanoma-Associated Antigen E), including MAGE 1 (e.g., GenBank Accession Nos. M77481 and AAA03229), MAGE 2 (e.g., GenBank Accession Nos. L18920 and AAA17729), MAGE 3 (e.g., GenBank Accession Nos. U03735 and AAA17446), MAGE 4 (e.g., GenBank Accession Nos. D32075 and A06841.1), etc.; any of the various tyrosinases (e.g., GenBank Accession Nos. U01873 and AAB60319); mutant ras; mutant p53 (e.g., GenBank Accession Nos. X54156, CAA38095 and AA494311); p97 melanoma antigen (e.g., GenBank Accession Nos. M12154 and AAA59992); human milk fat globule (HMFG) associated with breast tumors (e.g., GenBank Accession Nos. 556151 and AAB19771); any of the various BAGEs (Human B melanoma-Associated Antigen E), including BAGE1 (e.g., GenBank Accession No. Q13072) and BAGE2 (e.g., GenBank Accession Nos. NM_182482 and NP_872288), any of the various GAGEs (G antigen), including GAGE1 (e.g., GenBank Accession No. Q13065) or any of GAGE2-6; various gangliosides, CD25 (e.g., GenBank Accession Nos. NP_000408.1 and NM_000417.2).

Other tumor-specific antigens include the HPV 16/18 and E6/E7 antigens associated with cervical cancers (e.g., GenBank Accession Nos. NC_001526, FJ952142.1, ADB94605, ADB94606, and U89349), mucin (MUC 1)-KLH antigen associated with breast carcinoma (e.g., GenBank Accession Nos. J03651 and AAA35756), CEA (carcinoembryonic antigen) associated with colorectal cancer (e.g., GenBank Accession Nos. X98311 and CAA66955), gp100 (e.g., GenBank Accession Nos. 573003 and AAC60634) associated with for example melanoma, MARTI antigens associated with melanoma (e.g., GenBank Accession No. NP_005502), cancer antigen 125 (CA125, also known as mucin 16 or MUC16) associated with ovarian and other cancers (e.g., GenBank Accession Nos. NM_024690 and NP_078966); alpha-fetoprotein (AFP) associated with liver cancer (e.g., GenBank Accession Nos. NM_001134 and NP_001125); Lewis Y antigen associated with colorectal, biliary, breast, small-cell lung, and other cancers; tumor-associated glycoprotein 72 (TAG72) associated with adenocarcinomas; and the PSA antigen associated with prostate cancer (e.g., GenBank Accession Nos. X14810 and CAA32915).

Other exemplary tumor-specific proteins include, but are not limited to, PMSA (prostate membrane specific antigen; e.g., GenBank Accession Nos. AAA60209 and AAB81971.1) associated with solid tumor neovasculature, as well prostate cancer; HER-2 (human epidermal growth factor receptor 2, e.g., GenBank Accession Nos. M16789.1, M16790.1, M16791.1, M16792.1 and AAA58637) associated with breast cancer, ovarian cancer, stomach cancer and uterine cancer, HER-1 (e.g., GenBank Accession Nos. NM_005228 and NP_005219) associated with lung cancer, anal cancer, and gliobastoma as well as adenocarcinomas; NY-ESO-1 (e.g. GenBank Accession Nos. U87459 and AAB49693) associated with melanoma, sarcomas, testicular carcinomas, and other cancers, hTERT (aka telomerase) (e.g., GenBank Accession. Nos. NM_198253 and NP_937983 (variant 1), NM_198255 and NP_937986 (variant 2)); proteinase 3 (e.g., GenBank Accession Nos. M29142, M75154, M96839, X55668, NM 00277, M96628, X56606, CAA39943 and AAA36342), and Wilms tumor 1 (WT-1, e.g. GenBank Accession Nos. NM_000378 and NP_000369 (variant A), NM_024424 and NP_077742 (variant B), NM_024425 and NP_077743 (variant C), and NM_024426 and NP_077744 (variant D)).

In one example the tumor-specific protein is CD52 (e.g., GenBank Accession. Nos. AAH27495.1 and CAI15846.1) associated with chronic lymphocytic leukemia; CD33 (e.g., GenBank Accession. Nos. NM_023068 and CAD36509.1) associated with acute myelogenous leukemia; and CD20 (e.g., GenBank Accession. Nos. NP_068769 NP_031667) associated with Non-Hodgkin lymphoma.

In a specific example, the tumor-specific protein is CD44 (e.g., OMIM 107269, GenBank Accession. Nos. ACI46596.1 and NP_000601.3). CD44 is a marker of cancer stem cells and is implicated in intercellular adhesion, cell migration, cell spatial orientation, and promotion of matrix-derived survival signal. High expression of CD44 on the plasma membrane of tumors can be associated with tumor aggressiveness and poor outcome.

Thus, the disclosed methods can be used to treat any cancer that expresses a tumor-specific protein.

### Exemplary Antibody-IR700 Molecules

Because cell surface protein sequences are publically available (for example as described above), making or purchasing antibodies (or other small molecules that can be conjugated to IR700) specific for such proteins can be accomplished. For example, if the tumor-specific protein HER2 is selected as a target, antibodies specific for HER2 (such as Trastuzumab) can be purchased or generated and attached to the IR700 dye. Other specific examples are provided in Table 1. In one example, the antibody is a humanized monoclonal antibody. Antibody-IR700 molecules can be generated using methods such as those described in WO 2013/009475 (incorporated by reference herein in its entirety).

TABLE 1
Exemplary tumor-specific antigens and antibodies

| Tumor- | | |
|---|---|---|
| Specific | | Exemplary Antibody/Small |
| Antigen | Exemplary Tumors | Molecules |
| HER1 | Adenocarcinoma (e.g., | Cetuximab, panitumumab, |
| | colorectal cancer, head and | zalutumumab, nimotuzumab, |
| | neck cancer) | matuzumab, necitumumab, |
| | | imgatuzumab, 806. Small |
| | | molecule inhibitors gefitinib, |
| | | erlotinib, and lapatinib can |
| | | also be used. |
| HER2 | breast cancer, ovarian | Trastuzumab (Herceptin ®), |
| | cancer, stomach cancer, | pertuzumab (Perjeta ®, |
| | uterine cancer | Omnitarg ®) |
| HER3 | Breast, colon, lung, | Patritumab, Duligotumab, |
| | ovarian, prostate, and head | MM-121 |
| | and neck squamous cell | |
| | cancer | |
| CD19 | B cell lymphoma, CLL, | GBR 401, MEDI-551, |
| | ALL | Blinatumomab (Blincyto ®) |
| CD20 | Non-Hodgkin lymphoma | Tositumomab (Bexxar ®); |
| | | Rituximab (Rituxan, |
| | | Mabthera); Ibritumomab |
| | | tiuxetan (Zevalin, |
| | | for example in combination |
| | | with yttrium-90 or |
| | | indium-111 |
| | | therapy); Ofatumumab |
| | | (Arzerra ®), veltuzumab, |
| | | obinutuzumab, ublituximab, |
| | | ocaratuzumab |
| CD22 | Non-Hodgkin's | Narnatumab, inotuzumab |
| | lymphoma, CLL, hairy cell | ozogamicin, moxetumomab |
| | leukemia, ALL, solid | pasudotox |
| | tumors | |
| CD25 | T-cell lymphoma | Daclizumab (Zenapax), |
| | | Basiliximab |
| CD30 | Hodgkin's lymphoma | Brentuximab vedotin |
| | | (ADCETRIS ®), iratumumab |
| CD33 | Acute myelogenous | Gemtuzumab (Mylotarg, for |
| | leukemia | example in combination with |
| | | calicheamicin therapy); |
| | | lintuzumab |
| CD37 | CLL, non-Hodgkin | Otlertuzumab |
| | lymphoma, mantle cell | |
| | lymphoma | |
| CD38 | Multiple myeloma | Daratumumab |
| CD40 | Multiple myeloma, non- | Lucatumumab, dacetuzumab |
| | Hodgkin's or Hodgkin's | |
| | lymphoma | |
| CD44 | Cancer stem cells, breast, | bivatuzumab |
| | prostate, renal, head and | RG3756 |
| | neck cancer, lymphoma, | |
| | leukemia | |
| CD52 | chronic lymphocytic | Alemtuzumab (Campath) |
| | leukemia | |
| CD56 | Small cell lung cancer, | Lorvotuzumab mertansine |
| | ovarian cancer | |
| CD70 | Renal cell carcinoma | Vorsetuzumab mafodotin |
| CD74 | Multiple myeloma | Milatuzumab |
| CD140 | Glioblastoma, non-small | Tovetumab |
| | cell lung cancer | |
| CAIX | Renal cell carcinoma | Girentuximab, cG250 |
| CEA | colorectal cancer, some | Arcitumomab (CEA-scan |
| | gastric cancers, biliary | (Fab fragment, approved |
| | cancer | by FDA), colo101; |
| | | Labetuzumab |
| | | (CEA-Cide ®) |
| Cancer | ovarian cancer, | OC125 monoclonal antibody |
| antigen | mesothelioma, breast | |
| 125 (CA125) | cancer | |
| Alpha- | hepatocellular carcinoma | ⁹⁰Y-tacatuzumab tetraxetan; |
| fetoprotein | | ab75705 (available from |
| (AFP) | | Abcam) and other |
| | | commercially |
| | | available AFP antibodies |
| Cytokeratin | Colorectal cancer | ^{99m}Tc- Votumumab |
| | | (HUMASPECT ®) |
| EGFL7 | Non-small cell lung cancer, | Parsatuzumab |
| | colorectal cancer | |
| EpCAM | Epithelial tumors (breast, | IGN101, oportuzumab |
| | colon and lung) | monatox, tucotuzumab |
| | | celmoleukin, adecatumumab |
| EPHA3 | Lung, kidney and colon | KB004, IIIA4 |
| | tumors, melanoma, glioma | |
| | and hematological | |
| | malignancies | |
| FAP | Colon, breast, lung, | Sibrotuzumab, F19 |
| | pancreas, and head and | |
| | neck tumors | |
| Fibronectin | Hodgkin's lymphoma | Radretumab |
| Folate- | Ovarian cancer | MOv18 and MORAb-003 |
| binding | | (farletuzumab) |
| protein | | |
| Folate | Ovarian cancer | Farletuzumab |
| receptor | | |
| alpha | | |
| Frizzled | Breast, pancreatic, non- | Vantictumab |
| receptor | small cell lung cancer | |
| Gangliosides | Neuroectodermal tumors | 3F8, ch14.18, KW-2871 |
| (e.g., GD2, | and some epithelial tumors | |
| GD3 and | | |
| GM2) | | |
| gpA33 | Colorectal cancer | huA33 |
| HGF | Solid tumors | Rilotumumab, ficlatuzumab |
| IGF1R | Glioma, lung, breast, head | Cixutumumab, dalotuzumab, |
| | and neck, prostate and | figitumumab, ganitumab, |
| | thyroid cancer | robatumumab, |
| | | teprotumumab, |
| | | AVE1642, IMC-A12, |
| | | MK-0646, R1507, |
| | | and CP 751871 |
| IGLF2 | Breast cancer; | Dusigitumab |
| | Hepatocellular carcinoma; | |
| | Solid tumors | |
| IL-6 | renal cell cancer, prostate | Siltuximab |
| | cancer, Castleman's | |
| | disease | |
| Integrin αVβ3 | Tumor vasculature | Etaracizumab |
| | | (ABEGRIN ®), |
| | | intetumumab |
| Integrin α5β1 | Tumor vasculature | Volociximab |
| Lewis Y | colorectal cancer, biliary | B3 (Humanized), hu3S193, |
| | cancer | IgN311 |
| Mesothelin | Mesothelioma, pancreatic | Amatuximab |
| | cancer | |
| MET | Breast, ovarian, and lung | AMG 102, METMAB, SCH |
| | cancer | 900105 |
| Mucins | Breast, colon, lung and | Pemtumomab |
| | ovarian cancer | (THERAGYN ®), |
| | | cantuzumab mertansine, ⁹⁰Y |
| | | clivatuzumab tetraxetan, |
| | | oregovomab (OVAREX ®) |
| PDGFR-alpha | Soft tissue sarcoma | Olaratumab |
| Phosphatidyl- | Breast, pancreatic, | Bavituximab |
| serine | prostate, non-small cell | |
| | lung cancer, hepatocellular | |
| | carcinoma | |
| PSMA | Prostate cancer | J591 |
| RANKL | Prostate cancer, bone | Denosumab (XGEVAC ®) |
| | metastases | |
| Scatter factor | Non-small cell lung, | Onartuzumab |
| receptor | stomach, glioblastoma | |
| kinase | | |
| SLAMF7 | Multiple myeloma | Elotuzumab |
| (CD319) | | |
| Syndecan 1 | Multiple myeloma, breast, | Indatuximab ravtansine |
| | bladder cancer | |
| TAG72 | adenocarcinomas including | B72.3 (FDA-approved |
| | colorectal, pancreatic, | monoclonal antibody), CC49 |
| | gastric, ovarian, | (minretumomab) |
| | endometrial, mammary, | |
| | and non-small cell lung | |
| | cancer | |
| Tenascin | Glioma, breast and prostate | 81C6 |
| | tumors | |
| TRAILR1 | Colon, lung and pancreas | Mapatumumab (HGS-ETR1) |
| | tumors and hematological | |
| | malignancies | |
| TRAILR2 | Non-small cell lung cancer, | Conatumumab, |
| | non-Hodgkin's lymphoma, | lexatumumab, |
| | multiple myeloma | mapatumumab, |
| | | tigatuzumab, |
| | | HGS-ETR2, CS-1008 |
| Vascular | Colorectal cancer | Bevacizumab (Avastin ®) |
| endothelial | | |
| growth | | |
| factor | | |
| VEGFR | Epithelium-derived solid | IM-2C6, CDP791 |
| | tumors | |
| VEGFR2 | Gastric, non-small cell | Ramucirumab (Cyramza ™) |
| | lung, colorectal cancer | |
| Vimentin | Brain cancer | Pritumumab |

Additional antibodies that can be conjugated to IR700 include 3F8, Abagovomab, Afutuzumab, Alacizumab, Altumomab pentetate, Anatumomab mafenatox, Apolizumab, Bectumomab, Belimumab, Besilesomab, Capromab pendetide, Catumaxomab, Citatuzumab bogatox, Detumomab, Ecromeximab, Eculizumab, Edrecolomab, Epratuzumab, Ertumaxomab, Galiximab, Glembatumumab vedotin, Igovomab, Imciromab, Lumiliximab, Mepolizumab, Metelimumab, Mitumomab, Morolimumab, Nacolomab tafenatox, Naptumomab estafenatox, Nofetumomab merpentan, Pintumomab, Satumomab pendetide, Sonepcizumab, Taplitumomab paptox, Tenatumomab, TGN1412, Ticilimumab (tremelimumab), TNX-650, or Tremelimumab.

In one example, a patient is treated with at least two different antibody-IR700 molecules specific for cancer cell surface antigens. In one example, the two different antibody-IR700 molecules are specific for the same protein (such as HER-2), but are specific for different epitopes of the protein (such as epitope 1 and epitope 2 of HER-2). In another example, the two different antibody-IR700 molecules are specific for two different proteins or antigens. For example, anti-HER1-IR700 and anti-HER2-IR700 could be injected together as a cocktail to facilitate killing of cells bearing either HER1 or HER2.

In one specific example, the antibody-IR700 molecule is anti-CD44-IR700, such as RG7356-IR700 or bivatuzumab-IR700. RG7356 is a recombinant human antibody of the IgGl-kappa isotype that specifically binds to the constant region of the extracellular domain of the human cell-surface glycoprotein CD44 that is present on CD44 standard as well as on all CD44 splice variants. Bivatuzumab is a humanized mAb specific for CD44 v6.

### Immunomodulators

Immunomodulators of use in the disclosed methods include agents or compositions that activate the immune system and/or inhibit immuno-suppressor cells (also referred to herein as suppressor cells). Without being bound by theory, and as shown in FIG. 18, inhibition of immuno-suppressor cells and/or activation of immune responses increases tumor cell killing and also leads to production of memory T cells, which can provide a “vaccine” effect against recurrences and/or distant tumors or metastases.

In some embodiments, the immunomodulator is an inhibitor of immuno-suppressor cells, for example, an agent that inhibits or reduces activity of immuno-suppressor cells. In some cases, the immunomodulator kills immuno-suppressor cells. In some examples, the immuno-suppressor cells are regulatory T (Treg) cells. In some examples, not all of the suppressor cells are killed in vivo, as such could lead to development of autoimmunity. Thus, in some examples, the method reduces the activity or number of immuno-suppressor cells in an area of subject, such as in the area of a tumor or an area that used to have a tumor, by at least 50%, at least 60%, at least 75%, at least 80%, at least 90%, or at least 95%. In some examples, the method reduces the total number of suppressor cells in a subject by at least 50%, at least 60%, at least 75%, at least 80%, at least 90%, or at least 95%.

Inhibitors of immuno-suppressor cells include tyrosine kinase inhibitors (such as sorafenib, sunitinib, and imatinib), chemotherapeutic agents (such as cyclophosphamide or interleukin-toxin fusions, for example denileukin difitox (IL2-diphtheria toxin fusion), or anti-CD25 antibodies (such as daclizumab or basiliximab) or other antibodies that bind to suppressor cell surface proteins (such as those described below). In other examples, inhibitors of immuno-suppressor cells include immune checkpoint inhibitors, for example, anti-PD-1 or anti-PD-L1 antagonizing antibodies, thereby preventing PD-L1 from binding to PD-1 (referred to herein as PD-1/PD-L1 mAb-mediated immune checkpoint blockade (ICB)). Thus, in some examples, the immunomodulator is a PD-1 or PD-L1 antagonizing antibody, such as nivolumab, pembrolizumab, atezolizumab, avelumab, durvalumab, MPDL3280A, pidilizumab, CT011, AMP-224, AMP-514, MEDI-0680, BMS-936559, BMS935559, MEDI-4736, MPDL-3280A, MGA-271, indoximod, epacadostat, BMS-986016, MEDI-4736, MEDI-4737, MK-4166, BMS-663513, PF-05082566 (PF-2566), lirilumab, and MSB-0010718C. Checkpoint inhibitors also include anti-CTLA-4 antibodies, including ipilimumab and tremelimumab. The inhibitor of immuno-suppressor cells can also be a LAG-3 or B7-H3 antagonist, such as BMS-986016, and MGA271. In some examples, two or more of the inhibitors of immuno-suppressor cells can be administered to a subject. In one non-limiting example, a subject is administered an anti-PD1 and an anti-LAG-3 antibody.

In some examples, the agent that inhibits or reduces activity of suppressor cells includes one or more antibody-IR700 molecules, wherein the antibody specifically binds to a suppressor cell surface protein (such as CD25, CD4, C-X-C chemokine receptor type 4 (CXCR4), C-C chemokine receptor type 4 (CCR4), cytotoxic T-lymphocyte-associated protein 4 (CTLA4), glucocorticoid induced TNF receptor (GITR), OX40, folate receptor 4 (FR4), CD16, CD56, CD8, CD122, CD23, CD163, CD206, CD11b, Gr-1, CD14, interleukin 4 receptor alpha chain (IL-4Ra), interleukin-1 receptor alpha (IL-1Ra), interleukin-1 decoy receptor, CD103, fibroblast activation protein (FAP), CXCR2, CD33, and CD66b) and in some examples does not include a functional Fc region (e.g., consists of one or more F(ab′) 2 fragments). The presence of a functional Fc portion can result in autoimmune toxicity (such as antibody-dependent cell-mediated cytotoxicity (ADCC)). The result of ADCC is that too many suppressor cells may be killed, instead of only those suppressor cells exposed to the NIR light. Thus, the Fc portion of the antibody can be mutated or removed to substantially decrease its function (such as a reduction of at least 50%, at least 75% at least 80%, at least 90%, at least 95%, at least 99%, or 100% of the Fc function as compared to a non-mutated Fc region, such as an ability to bind to the Fcγ receptor). Methods and compositions for reducing activity of or killing suppressor cells are described in International Patent Publication No. WO 2017/027247 (incorporated herein by reference in its entirety).

In a non-limiting example, the immunomodulator is a CD25 antibody-IR700 molecule, such as daclizumab-IR700 or basiliximab-IR700.

In other embodiments, the immunomodulator is an immune system activator. In some examples, an immune system activator stimulates (activates) one or more T cells and/or natural killer (NK) cells. In one example, the immune system activator includes one or more interleukins (IL), such as IL-2, IL-15, IL-7, IL-12, and/or IL-21. In a non-limiting example, the immunomodulator includes IL-2 and IL-15. In another example, the immune system activator includes one or more agonists to co-stimulatory receptors, such as 4-1BB, OX40, or GITR. In a non-limiting example, the immunomodulator includes stimulatory anti-4-1BB, anti-OX40, and/or anti-GITR antibodies.

In some examples, one or more (such as 1, 2, 3, 4, 5, or more) doses of the immunomodulator is administered to the subject. Thus, administering the immunomodulator can be completed in a single day, or may be done repeatedly on multiple days with the same or a different dosage (such as administering at least 2 different times, 3 different times, 4 different times 5 different times or 10 different times). In some examples, the repeated administration are the same dose. In other examples, the repeated administrations are different does (such as a subsequent dose that is higher than the preceding dose or a subsequent dose that is lower than the preceding dose). Repeated administration of the immunomodulator may be done on the same day, on successive days, every other day, every 1-3 days, every 3-7 days, every 1-2 weeks, every 2-4 weeks, every 1-2 months, or at even longer intervals. In some examples, at least one dose of the immunomodulator is administered prior to irradiation and at least one dose of the immunomodulator is administered after irradiation.

### Irradiation

After the subject is administered one or more antibody-IR700 molecules, and after (or optionally before) the subject is administered one or more immunomodulators, the subject (or a tumor in the subject) is irradiated. As only cells expressing the cell surface protein will be recognized by the antibody, only those cells will have sufficient amounts of the antibody-IR700 molecules bound to it to kill the cells. This decreases the likelihood of undesired side effects, such as killing of normal cells, as the irradiation will only kill the cells to which the antibody-IR700 molecules are bound, not the other cells.

The subject (for example, a tumor in the subject) is irradiated with a therapeutic dose of radiation at a wavelength of 660-710 nm, such as 660-700 nm, 680-7000 nm, 670-690 nm, for example, 680 nm. In particular examples, the cells are irradiated at a dose of at least 1 J/cm², such as at least 10 J/cm², at least 30 J/cm², at least 50 J/cm², at least 100 J/cm², or at least 500 J/cm², for example, 1-1000 J/cm², 1-500 J/cm², 30-50 J/cm², 10-100 J/cm², or 10-50 J/cm².

The subject can be irradiated one or more times. Thus, irradiation can be completed in a single day, or may be done repeatedly on multiple days with the same or a different dosage (such as irradiation at least 2 different times, 3 different times, 4 different times 5 different times or 10 different times). In some examples, the repeated irradiations are the same dose. In other examples, the repeated irradiations are different does (such as a subsequent dose that is higher than the preceding dose or a subsequent dose that is lower than the preceding dose). Repeated irradiations may be done on the same day, on successive days, every other day, every 1-3 days, every 3-7 days, every 1-2 weeks, every 2-4 weeks, every 1-2 months, or at even longer intervals. In one example, a first irradiation is 50 J/cm² and a second irradiation is at 100 J/cm², where the irradiations are on consecutive days (for example, about 24 hours apart).

In some examples, the irradiation is provided with a wearable device incorporating an NIR LED. In other examples, another type of device that can be used with the disclosed methods is a flashlight-like device with NIR LEDs. Such a device can be used for focal therapy of lesions during surgery, or incorporated into endoscopes to apply NIR light to body surfaces after the administration of one or more PIT agents. Such devices can be used by physicians or qualified health personnel to direct treatment to particular targets on the body.

### Treatment Using Wearable NIR LEDs

As described herein, the disclosed methods are highly specific for cancer cells. However, in order to kill the cells circulating in the body or present on the skin, the patient can wear a device that incorporates an NIR LED. In some examples, the patient uses at least two devices, for example an article of clothing or jewelry during the day, and a blanket at night. In some example the patient uses at least two devices at the same time, for example two articles of clothing. These devices make it possible to expose the patient to NIR light using portable everyday articles of clothing and jewelry so that treatment remains private and does not interfere with everyday activities. In some examples, the device can be worn discreetly during the day for PIT therapy. Exemplary devices incorporating an NIR LED are disclosed in International Patent Application Publication No. WO 2013/009475 (incorporated by reference herein).

In one example, the patient is administered one or more antibody-IR700 molecules and one or more immunomodulators, using the methods described herein. The patient then wears a device that incorporates an NIR LED, permitting long-term therapy and treatment of tumor cells that are present in the blood or lymph or on the skin. In some examples, the dose is at least at least 1 J/cm², at least 10 J/cm², at least 20 J/cm², at least 30 J/cm², at least 40 J/cm², or at least 50 J/cm², such as 20 J/cm² or 30 J/cm². In some examples, administration of the antibody-IR700 molecule is repeated over a period of time (such as bi-weekly or monthly), to ensure therapeutic levels are present in the body.

In some examples, the patient wears or uses the device, or combination of devices, for at least 1 week, such as at least 2 weeks, at least 4 weeks, at least 8 weeks, at least 12 weeks, at least 4 months, at least 6 months, or even at least 1 year. In some examples, the patient wears or uses the device, or combination of devices, for at least 4 hours a day, such as at least 12 hours a day, at least 16 hours a day, at least 18 hours a day, or 24 hours a day. It is quite possible that multiple devices of a similar “everyday” nature (blankets, bracelets, necklaces, underwear, socks, shoe inserts) could be worn by the same patient during the treatment period. At night the patient can use the NIR LED blanket or other covering.

### Administration of Additional Therapies

As discussed above, prior to, during, or following administration of one or more antibody-IR700 molecules, immunomodulators, and/or irradiation, the subject can receive one or more other therapies. In one example, the subject receives one or more treatments to remove or reduce the tumor prior to administration of the antibody-IR700 molecules. In other examples, additional treatments or therapeutic agents (such as anti-neoplastic agents) can be administered to the subject to be treated, for example, after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation).

Examples of such therapies that can be used in combination with the disclosed methods, which enhance accessibility of the tumor to additional therapeutic agents for about 8 hours after the PIT, include but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. In some examples, the additional therapeutic agent is conjugated to a nanoparticle. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

“Microtubule binding agent” refers to an agent that interacts with tubulin to stabilize or destabilize microtubule formation thereby inhibiting cell division. Examples of microtubule binding agents that can be used in conjunction with the disclosed methods include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds can be used with the methods disclosed herein: suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevec® (imatinib), Iressa® (gefitinib), and Tarceva® (erlotinib) that prevent phosphorylation and activation of growth factors.

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof. For example, in some embodiments the anti-angiogenesis agent is an antibody that specifically binds to VEGF (e.g., Avastin, Roche) or a VEGF receptor (e.g., a VEGFR2 antibody). In one example the anti-angiogenic agent includes a VEGFR2 antibody, or DMXAA (also known as Vadimezan or ASA404; available commercially, e.g., from Sigma Corp., St. Louis, Mo.) or both. The anti-angiogenic agent can be bevacizumab, sunitinib, an anti-angiogenic tyrosine kinase inhibitors (TM), such as sunitinib, xitinib and dasatinib. These can be used individually or in any combination.

Other therapeutic agents, for example anti-tumor agents, that may or may not fall under one or more of the classifications above, also are suitable for administration in combination with the disclosed methods. By way of example, such agents include adriamycin, apigenin, rapamycin, zebularine, cimetidine, and derivatives and analogs thereof.

In some examples, the subject receiving the therapeutic antibody-IR700 molecule composition is also administered interleukin-2 (IL-2), for example via intravenous administration. In particular examples, IL-2 (Chiron Corp., Emeryville, Calif.) is administered at a dose of at least 500,000 IU/kg as an intravenous bolus over a 15 minute period every eight hours beginning on the day after administration of the antibody-IR700 molecules and continuing for up to 5 days. Doses can be skipped depending on subject tolerance.

Exemplary additional therapeutic agents include anti-neoplastic agents, such as chemotherapeutic and anti-angiogenic agents or therapies, such as radiation therapy. In one example the agent is a chemotherapy immunosuppressant (such as Rituximab, steroids) or a cytokine (such as GM-CSF). Chemotherapeutic agents are known in the art (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Combination chemotherapy is the administration of more than one agent to treat cancer.

Exemplary chemotherapeutic agents that can be used with the methods provided herein include but are not limited to, carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone, Doxil (liposome encapsulated doxiorubicine) and vinorelbine. Additional examples of chemotherapeutic agents that can be used include alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Specific non-limiting examples of alkylating agents are temozolomide and dacarbazine. Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone).

Examples of commonly used chemotherapy drugs include Adriamycin, Alkeran, Ara-C, BiCNU, Busulfan, CCNU, Carboplatinum, Cisplatinum, Cytoxan, Daunorubicin, DTIC, 5-fluoruracil (5-FU), Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

In some examples, the additional therapeutic agent is conjugated to (or otherwise associated with) a nanoparticle, such as one at least 1 nm in diameter (for example at least 10 nm in diameter, at least 30 nm in diameter, at least 100 nm in diameter, at least 200 nm in diameter, at least 300 nm in diameter, at least 500 nm in diameter, or at least 750 nm in diameter, such as 1 nm to 500 nm, 1 nm to 300 nm, 1 nm to 100 nm, 10 nm to 500 nm, or 10 nm to 300 nm in diameter).

In one example, at least a portion of the tumor (such as a metastatic tumor) is surgically removed (for example via surgical resection and/or cryotherapy), irradiated (for example administration of radioactive material or energy (such as external beam therapy) to the tumor site to help eradicate the tumor or shrink it), chemically treated (for example via chemoembolization) or combinations thereof, prior to administration of the disclosed therapies (such as administration of antibody-IR700 molecules and/or immunomodulators). For example, a subject having a metastatic tumor can have all or part of the tumor surgically excised prior to administration of the disclosed therapies. In an example, one or more chemotherapeutic agents are administered following treatment with antibody-IR700 molecules, immunomodulators, and irradiation. In another particular example, the subject has a metastatic tumor and is administered radiation therapy, chemoembolization therapy, or both concurrently with the administration of the disclosed therapies.

In some examples, the additional therapeutic agent administered is a monoclonal antibody, for example, 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

### Production of Memory T Cells

Also provided are methods of producing memory T cells specific for a target cell. In particular examples, the methods include administering to a subject a therapeutically effective amount of one or more antibody-IR700 molecules, where the antibody specifically binds to a target cell surface molecule, such as a tumor specific antigen (such as those listed in Table 1). The methods also include administering to the subject a therapeutically effective amount of one or more immunomodulators (such as an immune system activator or an inhibitor of immuno-suppressor cells), either simultaneously or substantially simultaneously with the antibody-IR700 molecules or sequentially (for example, within about 0 to 24 hours). In a specific example, the immunomodulatory agent is a PD-1 or PD-L1 antagonistic antibody. In another specific example, the immunomodulatory agent is a CD25 antibody-IR700 molecule. The subject or cells in the subject are then irradiated at a wavelength of 660 to 740 nm, such as 660 to 710 nm (for example, 680 nm) at a dose of at least 1 J/cm² (such as at least 50 J/cm² or at least 100 J/cm²).

Memory T cells may be either CD4+ or CD8+ and usually express CD45RO. Thus, in some examples, memory T cells are identified by detecting cells expressing CD45RO. A number of subtypes of memory T cells are known. For example, central memory T cells (T_{CM} cells) express CD45RO, C-C chemokine receptor type 7 (CCR7), and L-selectin (CD62L). Central memory T cells also have intermediate to high expression of CD44. This memory subpopulation is commonly found in the lymph nodes and in the peripheral circulation. Effector memory T cells (T_{EM} cells and TEMRA cells) express CD45RO, but lack expression of CCR7 and L-selectin. They also have intermediate to high expression of CD44. These memory T cells lack lymph node-homing receptors and are thus found in the peripheral circulation and tissues. T_{EMRA} cells also express CD45RA. Tissue resident memory T cells (T_{RM}) express integrin αeβ7. Specific to T_{RM}s are genes involved in lipid metabolism, being highly active, roughly 20- to 30-fold more active than in other types of T-cells. Stem memory (T_{SCM} cells) are CD45RO⁻, CCR7⁺, CD45RA⁺, CD62L⁺ (L-selectin), CD27⁺, CD28⁺ and IL-7Rα⁺, but they also express large amounts of CD95, CXCR3, and LFA-1.

In some examples, the disclosed methods increase memory T cells by at least 1% (for example, at least 2%, 5%, 7%, 10%, 20%, 30%, 40%, 50%, 60%, 70%, 80%, 90%, 95%, 2-fold, 3-fold, 4-fold, 5-fold, 10-fold, or more) compared to the amount of memory T cells in a subject who has not been treated with the disclosed methods. In some examples, total memory T cells are increased, while in other examples, one or more subtypes of memory T cells are increased compared to an untreated subject. In other examples, the methods increase memory T cells for a specific antigen, such as a tumor-specific antigen, by at least 1% (for example, at least 2%, 5%, 7%, 10%, 20%, 30%, 40%, 50%, 60%, 70%, 80%, 90%, 95%, 2-fold, 3-fold, 4-fold, 5-fold, 10-fold, or more) compared to the amount of memory T cells in a subject who has not been treated with the disclosed methods. In non-limiting examples, the memory T cells recognize one or more of p15E, birc5, twist, and p53 (see Example 3).

The number and/or type of memory T cells can be determined in a sample from a subject (such as a treated subject). In some examples, immunoassays and/or genetic analysis are used to detect memory T cells in a blood sample from a subject. For example, presence and/or amount of one or more memory T cell surface markers can be measured, for example by flow cytometry. In another example, tumor infiltrating lymphocytes (TIL) can be obtained from a treated subject, and checked for functional reactivity against antigens, such as tumor-specific antigens. Exemplary methods are provided in Example 3, below. The number, type, and/or reactivity profile of memory T cells can be compared to a control, such as an untreated subject, the subject prior to treatment, and/or a reference number (such as an average obtained from a population of normal (e.g., healthy) individuals).

### Production of Polyclonal Antigen-Specific TIC

Also provided are methods of increasing polyclonal antigen-specific TIC responses against MHC type I-restricted tumor specific antigens. In particular examples, the methods include administering to a subject a therapeutically effective amount of one or more antibody-IR700 molecules, where the antibody specifically binds to a target cell surface molecule, such as a tumor specific antigen (such as those listed in Table 1). The methods also include administering to the subject a therapeutically effective amount of one or more immunomodulators (such as an immune system activator or an inhibitor of immuno-suppressor cells), either simultaneously or substantially simultaneously with the antibody-IR700 molecules or sequentially (for example, within about 0 to 24 hours). In a specific example, the immunomodulatory agent is a PD-1 or PD-L1 antagonistic antibody. In another specific example, the immunomodulatory agent is a CD25 antibody-IR700 molecule. The subject or cells in the subject are then irradiated at a wavelength of 660 to 740 nm, such as 660 to 710 nm (for example, 680 nm) at a dose of at least 1 J/cm² (such as at least 50 J/cm² or at least 100 J/cm²).

### Example 1

### Materials and Methods

This example describes materials and methods used to obtain the results in Examples 2-9 (see also Nagaya et al., Cancer Immunol. Res. 7:401-13, 2019, herein incorporated by reference in its entirety).

### Reagents

Water soluble, silica-phthalocyanine derivative, IRDye 700DX NHS ester (IR700) was from LI-COR Biosciences (Lincoln, Nebr., USA). Anti-mouse/human CD44-specific mAb (clone IM7) and an anti-mouse PD-1 (CD279) specific mAb (clone RMP1-14) were from BioXCell (West Lebanon, N.H., USA). All other chemicals were of reagent grade.

### Synthesis of IR700-Conjugated Anti-CD44 mAb

Anti-CD44 mAb (1.0 mg, 6.7 nmol) was incubated with IR700 NHS ester (65.1 μg, 33.3 nmol) in 0.1 M Na₂HPO₄ (pH 8.6) at room temperature for 1 h and purified with a Sephadex G25 column (PD-10; GE Healthcare, Piscataway, N.J., USA). Protein concentration was determined with Coomassie Plus protein assay kit (Thermo Fisher Scientific Inc, Rockford, Ill., USA) by measuring the absorption at 595 nm with UV-Vis (8453 Value System; Agilent Technologies, Santa Clara, Calif., USA). IR700 concentration was measured by absorption at 689 nm to confirm the number of fluorophore molecules per mAb. CD44-IR700 conjugate synthesis was controlled so that an average of two IR700 molecules were bound to each CD44 antibody. Fluorescence at 700 nM and molecular weight of CD44-IR700 conjugates was verified using sodium dodecyl sulfate-polyacrylamide (4-20% gradient) gel electrophoresis (SDS-PAGE).

### Cell Culture

MC38 (colon cancer) cells stably expressing luciferase (MC38-luc), LLC (Lewis lung carcinoma) cells, and MOC1 (murine oral carcinoma) cells were maintained in culture as previously described (Farsaci et al., Cancer Immunol Res. 2014; 2:1090-102; Hodge et al., Cancer Biother Radiopharm. 2012; 27:12-22; Judd et al., Cancer Res. 2012; 72:365-74). Cells were maintained in culture for no more than 30 passages and routinely tested negative for mycoplasma.

### In Vitro NIR-PIT

MC38-luc, LLC or MOC1 cells (2×10⁵) were seeded into 12 well plates, incubated for 24 h, then exposed to media containing 10 μg/mL of CD44-IR700 for 6 h at 37° C. Cells were irradiated with a red light-emitting diode (LED, 690±20 nm wavelength, L690-66-60; Marubeni America Co., Santa Clara, Calif., USA) at a power density of 50 mW/cm². Cells were harvested with a cell scraper, stained with propidium iodide (PI, 2 μg/mL) at room temperature for 30 min, then analyzed on a BD FACSCalibur (BD Biosciences) using CellQuest software.

### Animal and Tumor Models

Six to eight-week-old female wild-type C57BL/6 mice (strain #000664) were from Jackson Laboratory (Sacramento, Calif., USA). Mice were shaved at sites of subcutaneous tumor transplantation prior to injection. Tumors were established via subcutaneous injection of 6×10⁶ cells for each model. In some experiments, multiple MC38 tumors were established. Established tumors were treated at volumes of approximately 50 mm³ (4 to 5 mm in diameter; day 4 for MC38-luc and LLC tumors; day 18 for MOC1 tumors). For NIR-PIT treatments and fluorescence/bioluminescence imaging (BLI), mice were anesthetized with inhaled 3-5% isoflurane and/or via intraperitoneal injection of 1 mg of sodium pentobarbital (Nembutal Sodium Solution, Ovation Pharmaceuticals Inc., Deerfield, Ill., USA). CD44-IR700 was administered via IV (tail-vein) injection and NIR light was administered at 50 J/cm² on day 5 and 100 J/cm² on day 6. Previous results demonstrated that two NIR light doses kill up to 80% of target-expressing cells (Mitsunaga et al., Nat Med. 2011; 17:1685-91; Nagaya et al., Mol Cancer Res. 2017; 15:1667-77). For mice bearing multiple tumors, tumors not exposed to NIR were shielded from NIR light exposure with aluminum foil. PD-1 mAb was administered via intraperitoneal injection using standard technique. Tumor volumes were based on caliper measurements (tumor volume=length×width²×0.5). In some MC38 experiments, mice cured after combination NIR-PIT and PD-1 mAb treatment were challenged via subcutaneous injection of MC38 (6×10⁶) cells in the contralateral flank. Tumor volume and animal weight was measured three times a week for MC38-luc and LLC tumors and two times a week for MOC1 tumor until the tumor volume reached 2000 mm³, whereupon the mice were euthanized with inhalation of carbon dioxide gas. For all immune correlative experiments, mice were euthanized via awake cervical dislocation.

### Fluorescence Imaging

In vitro, MC38-luc, LLC or MOC1 cells (1×10⁴) were seeded on cover-glass-bottom dishes, incubated for 24 h, then exposed to 10 μg/mL CD44-IR700 for 6 h at 37° C. Cells were then analyzed via fluorescence microscopy (BX61; Olympus America, Inc., Melville, N.Y., USA) using a 590-650 nm excitation filter and a 665-740 nm band pass emission filter. Transmitted light differential interference contrast (DIC) images were also acquired. In vivo, IR700 fluorescence and white light images were obtained using a Pearl Imager (700 nm fluorescence channel) and analyzed using Pearl Cam Software (LICOR Biosciences, Lincoln, Nebr.). Regions of interest (ROIs) within the tumor were compared to adjacent non-tumor regions as background (left dorsum). Average fluorescence intensity of each ROI was calculated. (n 10).

### Bioluminescence Imaging (BLI)

In vitro, MC38-luc cells were seeded into 12 well plates (2×10⁵ cells/well) or a 10 cm dish (2×10⁷ cells), incubated for 24 h, then exposed to 10 μg/mL of CD44-IR700 for 6 h at 37° C. Cells were treated with LED or NIR laser light (690±5 nm, BWF5-690-8-600-0.37; B&W TEK INC., Newark, Del., USA) in phenol-red-free culture medium. For luciferase activity, cells were exposed to 150 μg/mL D-luciferin (Gold Biotechnology, St. Louis, Mo., USA) 1 h after NIR-PIT treatment, and luciferase activity (photons/min) was obtained on a BLI system (Photon Imager; Biospace Lab, Paris, France) using M3 Vision Software (Biospace Lab). In vivo, D-luciferin (15 mg/mL, 200 μL) was injected intraperitoneally and the mice were analyzed on a BLI system (Photon Imager) for luciferase activity (photons/min/cm²). ROIs were set to include the entire tumor with the adjacent non-tumor region as background.

### Histological Analysis

Tumors (day 10 for MC38-luc and LLC tumors, day 24 for MOC1 tumors) were excised, formalin-fixed and paraffin embedded, and sectioned at 10 μm. Following standard H&E staining, sectioned were analyzed on an Olympus BX61 microscope.

### Immunofluorescence

Formalin fixed paraffin embedded sections were stained as described (18). Briefly, sections were deparaffinized in an ethanol gradient, then blocked in separate incubations with bloxall (Vector Laboratories), 2.5% normal goat serum (Vector Laboratories) and Renaissance Ab diluent (Biocare Medical). Primary antibody targeting CD4 (Invitrogen, clone 4SM95, 1:75 dilution) in Renaissance Ab diluent was added for 45 minutes on an orbital shaker. Slides were washed five times then stained with an anti-rat secondary antibody (Vector Laboratories). Following four more washes, slides were stained with TSA conjugated Opa1650 (Perkin Elmer, 1:150 dilution) in Amplification plus buffer (Perkin Elmer). Slides were washed four times with 1×TBS-T. Slides were washed, exposed to antigen stripping buffer (0.1 M glycine pH10+0.5% tween 20), and re-blocked as above. Primary antibody targeting CD8 (Invitrogen, clone 4SM15, 1:75 dilution) in Reinassance Ab diluent was added for 45 minutes. A nti-rat secondary antibody (Vector Laboratories) was added as above. Following four more washes, slides were stained with TSA conjugated Opa1520 (Perkin Elmer, 1:150 dilution) in Amplification plus buffer (Perkin Elmer). Nuclei counter-staining was achieved with Spectral DAPI (Perkin Elmer, 1:500). Slides were rinsed once with ddH2O, coverslipped with Vectashield hard mount (Vector Laboratories) and sealed with nail polish.

### Flow Cytometry

In vitro, MC38-luc, LLC or MOC1 cells (2×10⁵) were seeded into 12 well plates and incubated for 24 h then exposed to media containing 10 μg/mL of CD44-IR700 for 6 h at 37° C. Cells were harvested and analyzed on a BD FACSCalibur (BD Biosciences) using CellQuest software. To validate specific binding of CD44-IR700, cells were incubated with excess unconjugated CD44 antibody (100 μg) prior to incubation with CD44-IR700. In vivo, tumors were harvested (day 10 for MC38-luc and LLC tumors, day 24 for MOC1 tumors) and immediately digested as previously described (Moore et al., Cancer Immunol Res. 2016; 4:1061-71). Following FcγR (CD16/32) block, single cell suspensions were stained with primary antibodies. Suspensions were stained with fluorophore-conjugated primary antibodies including anti-mouse CD45.2 clone 104, CD3 clone 145-2C11, CD8 clone 53-6.7, CD4 clone GK1.5, PD-1 clone 29F.1A12, CD11c clone N418, F4/80 clone BM8, CD11b clone M1/70, Ly-6C clone HK1.4, Ly-6G clone 1A8, I-A/I-E clone M5/114.15.2, PD-L1 clone 10F.9G2, CD25 clone PC61.5.3, CTLA-4 clone UC10-4B9, CD31 clone 390, PDGFR clone APAS and CD44 clone IM7 (Biolegend) for one hour in a 1% BSA/1×PBS buffer. Suspensions were washed, stained with a viability marker (7AAD or zombie dye; Biolegend) and analyzed by flow cytometry on a BD Canto using BD FACS Diva software. Isotype controls and “fluorescence minus one” methods were used to validate staining specificity. FoxP3⁺ regulatory CD4+T-lymphocytes (Le_{g}s) were stained using the Mouse Regulatory T Cell Staining Kit #1 (eBioscience) per manufacturer protocol. Post-acquisition analysis was performed with FlowJo vX10.0.7r2.

### Antigen-Specific TIL Reactivity

Minced fragments of fresh tumor were incubated in RPMI 1640-based media supplemented with glutamine, HEPES, nonessential amino acids, sodium pyruvate, β-mercaptoethanol, 5% FBS, and 100 U/mL recombinant murine IL-2 for 72 hours to extract TIL. Untouched TIL were enriched with negative magnetic sorting (AutoMACSpro, Miltenyi Biotec). Antigen presenting cells (APC; splenocytes from naive, WT B5 mice irradiated to 50Gy) were pulsed for one hour with peptides of interest including class I-restricted antigens p15E₆₀₄₋₆₁₁ (H-2K^{b}-restricted KSPWFTTL), Survivin/Birc5₅₇₋₆₄ (H-2K^{b}-restricted QCFFCFKEL), Twist₁₂₅₋₁₃₃ (H-2D^{b}-restricted TQSLNEAFA), and Trp5₃₂₃₂₋₂₄₀ (H-2D^{b}-restricted KYMCNSSCM) Antigen-pulsed APC and TIL were co-incubated for 24 hours at a 3:1 APC:TIL ratio. Supernatants were analyzed for IFNγ production by ELISA (R&D) per manufacturer recommendations. TIL alone, APC alone, and peptide stimulations with Ovalbumin₂₅₇₋₂₆₄ (H-2K^{b}-restricted SIINFEKL) and VSV-N₅₂₋₅₉ (H-2Db-restricted RGYVYQGL) were used as controls.

### RT-PCR

RNA from whole tumor lysates was purified using the RNEasy Mini Kit (Qiagen) per the manufacturer's protocol. cDNA was synthesized utilizing a high capacity cDNA reverse transcription kit with RNase inhibitor (Applied Biosystems). A Taqman Universal PCR master mix was used to assess the relative expression of target genes compared to GAPDH on a Viia7 qPCR analyzer (Applied Biosystems). Custom primers were designed to flank nucleotide regions encoding the MHC class I-restricted epitopes for each tumor associated antigen.

### Statistical Analysis

Data are expressed as means±SEM from a minimum of five experiments, unless otherwise indicated. Statistical analyses were carried out using GraphPad Prism version 7 (GraphPad Software, La Jolla, Calif., USA). Student's t test was used to compare the treatment effects with that of control in vitro. To compare tumor growth in a re-inoculated tumor model of MC38-luc, the Mann Whitney test was used. For multiple comparisons, a one-way analysis of variance (ANOVA) followed by the Tukey's test was used. The cumulative probability of survival based on volume (2000 mm³) were estimated in each group with a Kaplan-Meier survival curve analysis, and the results were compared with use of the log-rank test. A p-value of <0.05 was considered statistically significant.

### Example 2

### In Vitro Effects of NIR-PIT on Cancer Cells

MC38-luc is a mouse colon cancer cell line expressing luciferase under control of the CMV promoter (Zabala et al., Mol. Cancer 8:2, 2009). LLC (Lewis lung carcinoma) cells and MOC1 (murine oral carcinoma) cells were also used. Anti-CD44-IR700 was produced using the methods described in WO 2013/009475 (incorporated by reference herein). Briefly, anti-CD44 mAb (1.0 mg, 6.7 nmol, clone IM7 from BioXCell, West Lebanon, N.H.) was incubated with IR700 NHS ester (65.1 μg, 33.3 nmol) in 0.1 M Na2HPO4 (pH 8.6) at room temperature for 1 h and purified with a Sephadex G25 column (PD-10; GE Healthcare, Piscataway, N.J., USA). CD44-IR700 conjugate synthesis was controlled so that an average of two IR700 molecules were bound to each CD44 antibody. Conjugates demonstrated strong fluorescent intensity and peak absorbance around 690 nm.

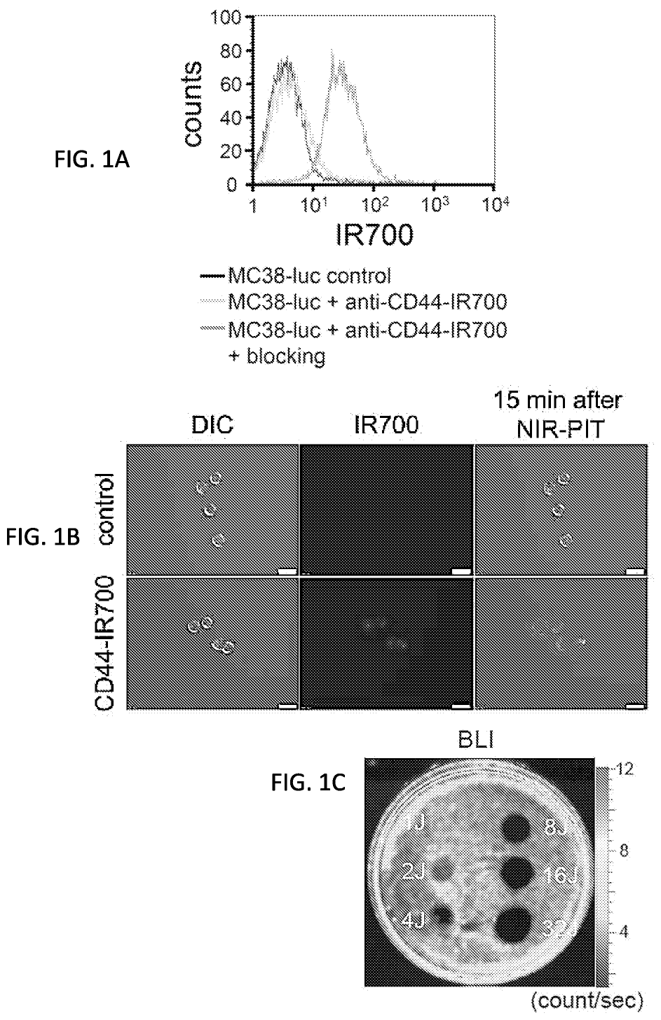

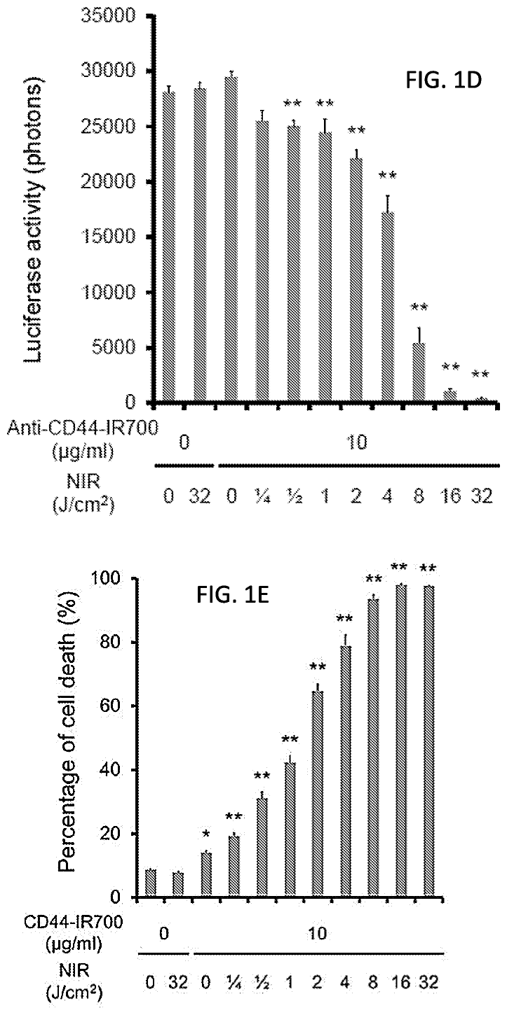

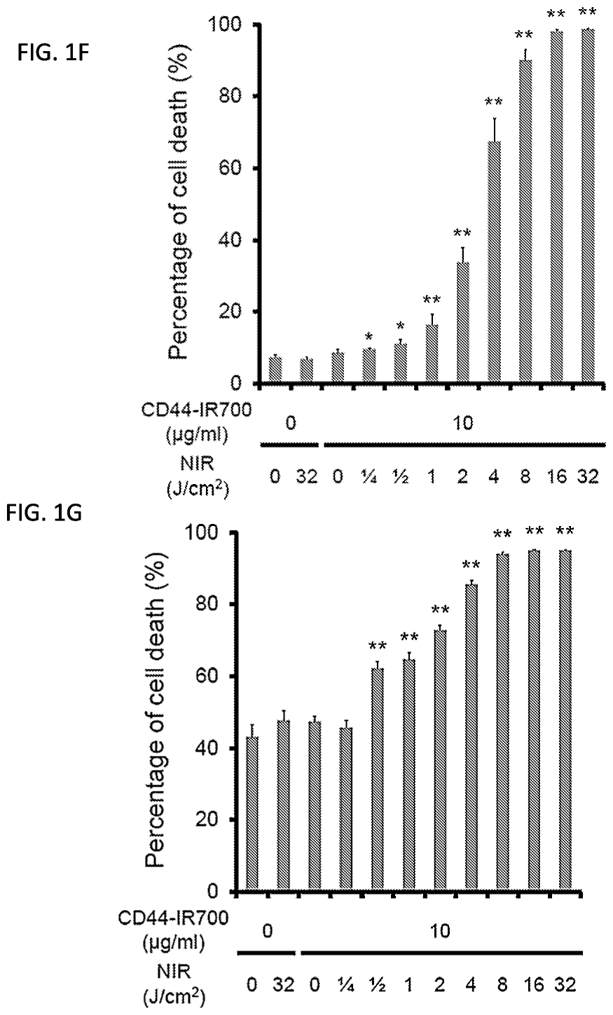

The effect of anti-CD44-IR700 on MC38-luc cells was evaluated in vitro. To verify anti-CD44-IR700 binding, fluorescence from cells after incubation with anti-CD44-IR700 was measured using a flow cytometer (FACS Calibur, BD BioSciences) and CellQuest software (BD BioSciences). MC38-luc cells were seeded into 12-well plates and incubated for 24 hours. Medium was replaced with fresh culture medium containing 10 mg/mL of anti-CD44-IR700 and incubated for 6 hours at 37° C. To validate the specific binding of the conjugated antibody, excess antibody (100 mg) was used to block 10 mg of anti-CD44-IR700 (FIG. 1A).

To detect the antigen specific localization and effect of NIR-PIT, fluorescence microscopy was performed (BX61; Olympus America, Inc.). MC38-luc, LLC or MOC1 cells (1×10⁴) were seeded on cover-glass-bottomed dishes and incubated for 24 hours. Anti-CD44-IR700 was then added to the culture medium at 10 mg/mL and incubated for 6 hours at 37° C. After incubation, the cells were washed with phosphate buffered saline (PBS). The filter set to detect IR700 consisted of a 590 to 650 nm excitation filter, a 665 to 740 nm band pass emission filter. Transmitted light differential interference contrast (DIC) images were also acquired. FIG. 1B is a digital image showing differential interference contrast (DIC) and fluorescence microscopy images of control and anti-CD44-IR700 treated MC38-luc cells. Necrotic cell death was observed upon excitation with NIR light in treated cells. This signal was completely reversed in the presence of excess unconjugated CD44 mAb, verifying binding specificity. NIR light exposure of tumor cells exposed to CD44-IR700 induced immediate cellular swelling, bleb formation, and rupture of vesicles indicative of necrotic cell death in all three cell lines (MC38-luc, LLC, and MOC1). These morphologic changes were observed within 15 min of NIR exposure (FIG. 1B).

For bioluminescence imaging (BLI), MC38-luc cells were seeded into 12 well plates (2×10⁵ cells/well) or a 10 cm dish (2×10⁷ cells) were seeded onto a 10-cm dish and preincubated for 24 hours. After replacing the medium with fresh culture medium containing 10 mg/mL of anti-CD44-IR700, the cells were incubated for 6 hours at 37° C. in a humidified incubator. After washing with PBS, phenol-red-free culture medium was added. Then, cells were exposed with a LED or a NIR laser which emits light at a 685 to 695 nm wavelength (BWF5-690-8-600-0.37; B&W TEK INC.) in phenol-red-free culture medium. The output power density in mW/cm² was measured with an optical power meter (PM 100, Thorlabs). FIG. 1C is a digital image of bioluminescence imaging (BLI) of a 10-cm dish showing NIR-light dose dependent luciferase activity in MC38-luc cells.

For luciferase activity (FIG. 1D), 150 mg/mL D-luciferin-containing media (Gold Biotechnology) was administered to PBS-washed cells 1 hour after NIR-PIT and images were obtained on a BLI system (Photon Imager; Biospace Lab). Regions of interest (ROI) were placed on each entire well, and the luciferase activity (photons/min) was then calculated using M3 Vision Software (Biospace Lab).

The cytotoxic effects of NIR-PIT with anti-CD44-IR700 were determined by flow cytometric propidium iodide (PI; Life Technologies) staining, which can detect compromised cell membranes. Two hundred thousand MC38-luc cells were seeded into 12-well plates and incubated for 24 hours. Medium was replaced with fresh culture medium containing 10 mg/mL of anti-CD44-IR700 and incubated for 6 h at 37° C. After washing with PBS, PBS was added, and cells were irradiated with a red light-emitting diode (LED), which emits light at 670 to 710 nm wavelength (L690-66-60; Marubeni America Co.) at a power density of 50 mW/cm² as measured with an optical power meter (PM 100, Thorlabs). Cells were scratched 1 hour after treatment. PI was then added in the cell suspension (final 2 mg/mL) and incubated at room temperature for 30 minutes, followed by flow cytometry. Each value represents mean±SEM of five experiments. FIG. 1E shows percentage of cell death in MC38-luc cells treated with NIR with or without 10 μg/ml CD44-IR700, measured with dead cell count using propidium iodide (PI) staining.

Bioluminescence imaging demonstrated decreased luciferase activity in a light-dose dependent manner (FIGS. 1C, 1D) in MC38-luc cells. Based on incorporation of propidium iodine (e.g., membrane permeability), NIR induced cell death in a light-dose dependent manner in MC38-luc (FIG. 1E), LLC (FIG. 1F) and MOC1 (FIG. 1G) cells exposed to CD44-IR700. NIR or CD44-1R700 alone did not induce significant alterations in cell viability.

These data demonstrate that NIR-PIT targeting CD44 induced specific cell death in MC38-luc, LLC and MOC1 cells in vitro.

### Example 3

### CD44 Expression within MOC1, LLC and MC38-Luc Tumor Compartments

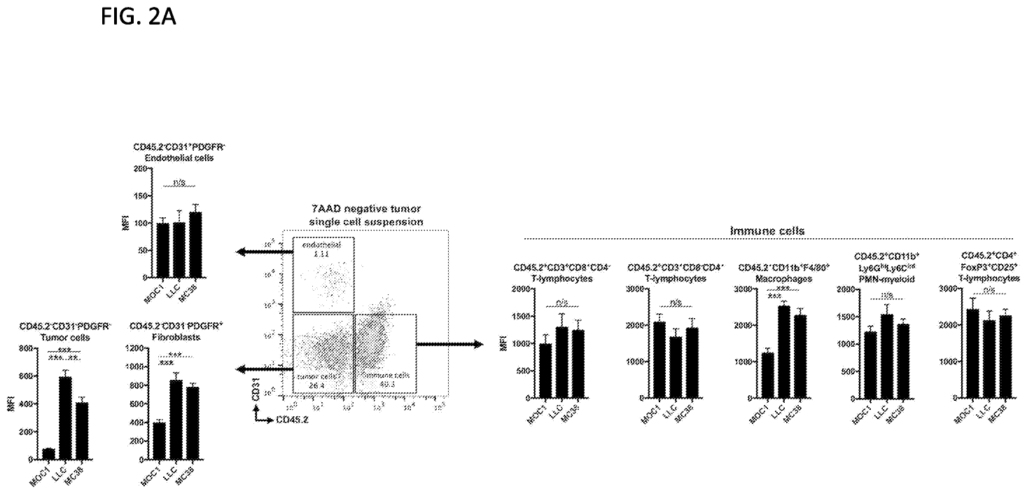

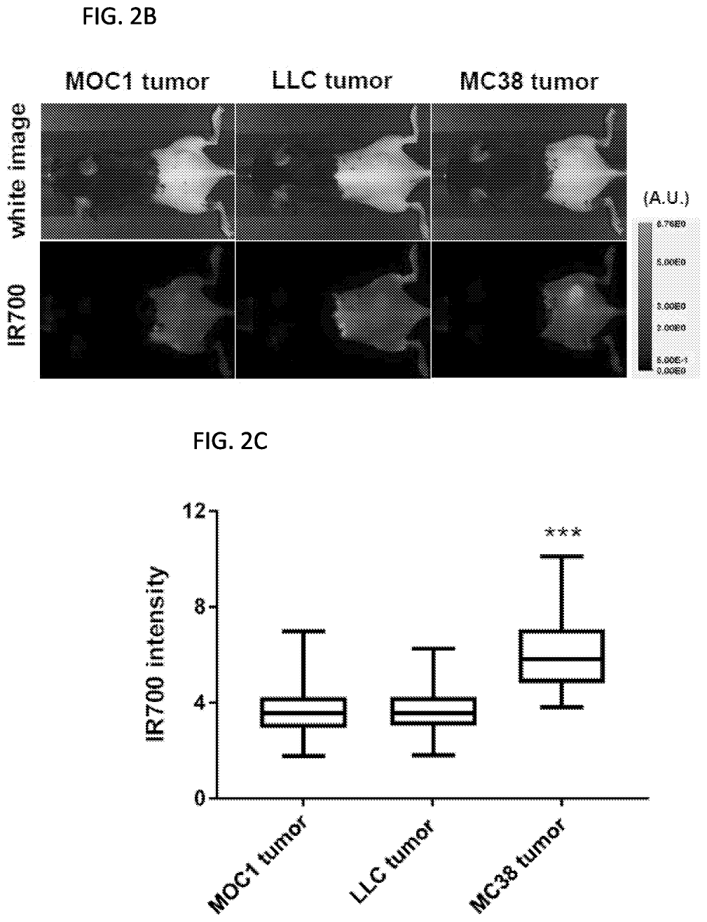

To verify target expression of CD44 in vivo, size matched MOC1 (day 24), LLC (day 10) and MC38 (day 10) tumors were assessed for CD44 expression within different tumor compartments via flow cytometry (FIG. 2A). Significant heterogeneity in tumor and stromal cell-specific CD44 expression was observed, with LLC and MC38-luc tumor cells expressing significantly greater levels of CD44 compared to MOC1. Expression of CD44 on immune cell subsets was more homogeneous between MOC1, LLC and MC38-luc tumors and was greater than CD44 expression on tumor and stromal cells on a cell-by-cell basis as measured by mean fluorescence intensity (MFI). Whole tumor accumulation of CD44-IR700 one day after injection, which is dependent upon multiple factors including target antigen expression and vascularity, was significantly greater in MC38-luc tumors (p<0.001) compared to LLC or MOC1 tumors (FIGS. 2B, 2C).

### Example 4

### In Vivo Effects of NIR-PIT and PD-1 mAb on Tumors

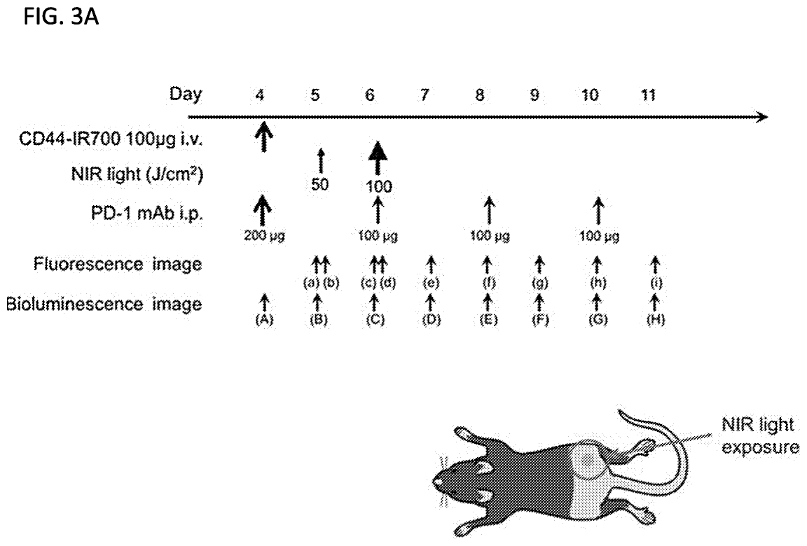

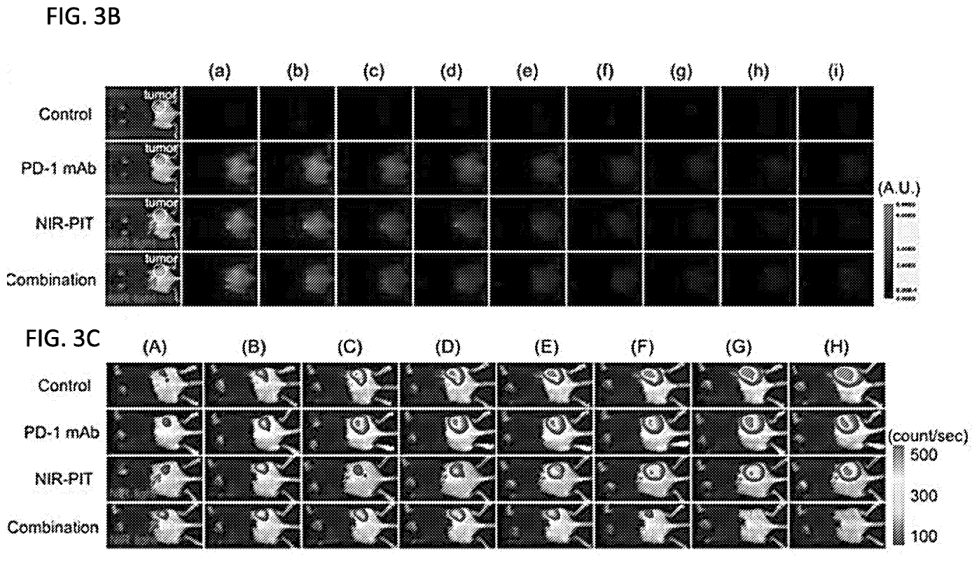

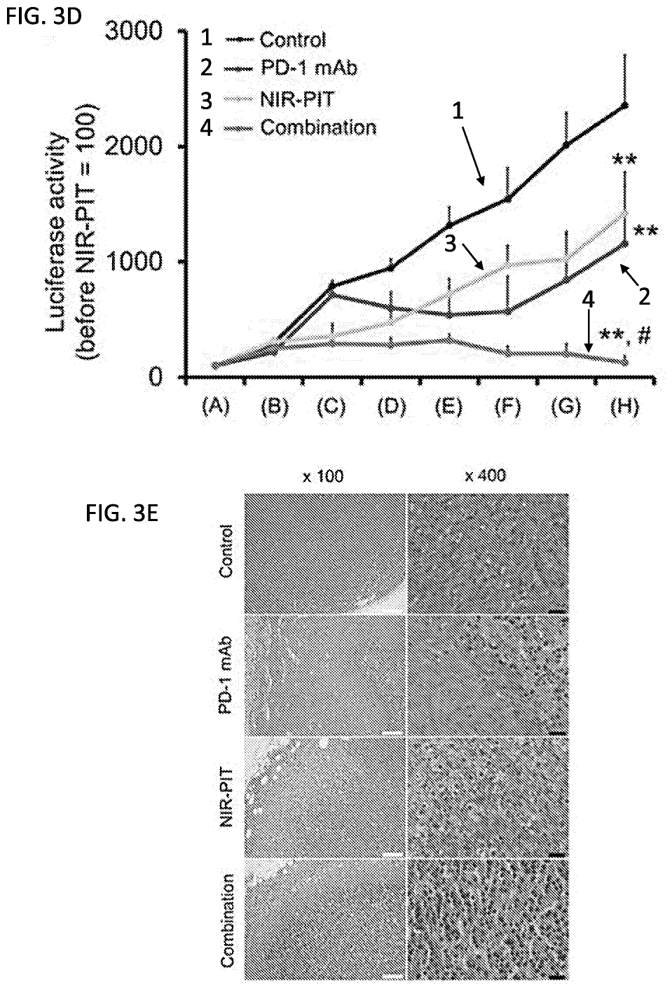

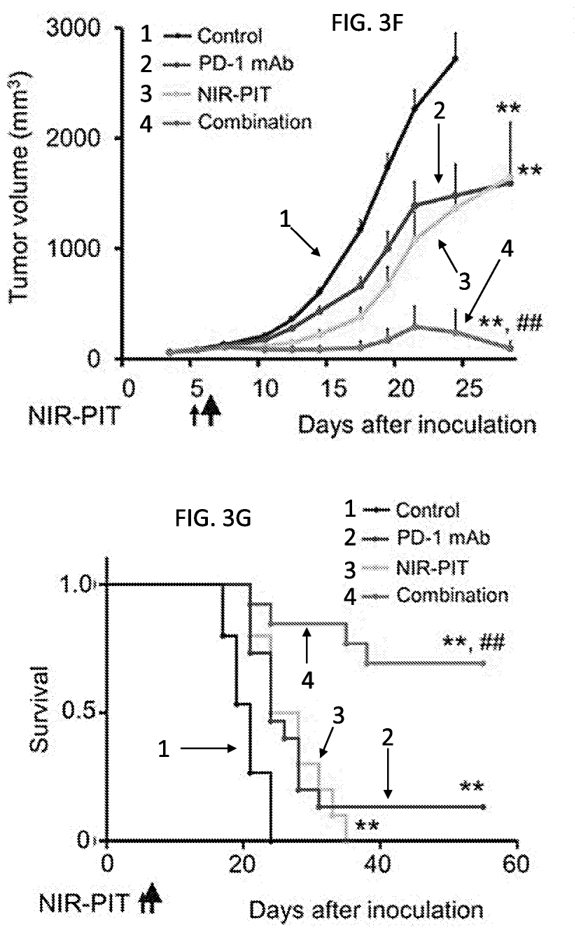

The effect of a combination therapy with anti-CD44-IR700 and anti-PD1 was tested in unilateral, bilateral, and multiple tumor models in mice. FIG. 3A shows the treatment scheme for a unilateral MC38-luc tumor in mice (10-13 mice in each group). Mice were unilaterally injected in the flank with 6 million tumor cells (day 0). Established tumors were treated at volumes of approximately 50 mm³ (4 to 5 mm in diameter; day 4 for MC38-luc and LLC tumors; day 18 for MOC1 tumors). On day 4, the mice were administered 100 μg anti-CD44-IR700 i.v. (tail vein) alone, or in combination with 200 μg anti-PD1 i.p. (within 1 hour of one another) (anti-mouse PD-1 (CD279) specific mAb (clone RMP1-14) from BioXCell (West Lebanon, N.H., USA)) and were subsequently administered 100 μg anti-PD1 i.p. on days 6, 8, and 10. NIR-PIT was performed on days 5 (50 J/cm²) and 6 (100 J/cm²). For mice bearing multiple tumors, tumors not exposed to NIR were shielded from NIR light exposure with aluminum foil.

Tumors were monitored by fluorescence imaging and bioluminescence imaging (FIG. 3A). in vivo IR700 fluorescence images were obtained with a Pearl Imager (LI-COR Biosciences) with a 700-nm fluorescence channel A ROI was placed on the tumor and the average fluorescence intensity of IR700 signal was calculated for each ROI using a Pearl Cam Software (LICOR Biosciences). For in vivo BLI, D-luciferin (15 mg/mL, 200 μL) was injected i.p., and the mice were analyzed on a BLI system (Photon Imager) for luciferase activity. ROIs were set to include the entire tumor in order to quantify BLI. ROIs were also placed in the adjacent non-tumor region as background (photons/min/cm²). Average luciferase activity of each ROI was calculated.

To detect the antigen-specific microdistribution in the tumor, fluorescence microscopy was performed. Tumor xenografts were excised (day 10 for MC38-luc and LLC tumors, day 24 for MOC1 tumors) from the right flank xenograft without treatment. Extracted tumors were frozen with optimal cutting temperature (OCT) compound (SAKURA Finetek Japan Co.) and frozen sections (10 μm thick) prepared. Fluorescence microscopy was performed using an Olympus BX61 microscope with the following filters: excitation wavelength 590 to 650 nm, emission wavelength 665 to 740 nm long pass for IR700 fluorescence. DIC images were also acquired. To evaluate histological changes, light microscopy was performed using Olympus BX61. Tumor xenografts were excised from mice without treatment, 24 hours after injection of anti-CD44-IR700 (i.v.) and 24 hours after NIR-PIT. Tumors were also excised from mice with bilateral flank tumors (both treated right-sided tumors and untreated left-sided tumors) 24 hours after NIR-PIT of the right tumor. Extracted tumors were also placed in 10% formalin, and serial 10-mm slice sections were fixed on a glass slide with H&E staining.

Compared to control or PD-1 mAb alone groups, NIR-PIT resulted in a near-immediate decrease in tumor fluorescence signal, likely due to dispersion of IR700 from dying cells (FIG. 3B). Combination NIR-PIT and PD-1 mAb treatment resulted in dramatically decreased bioluminescence compared to control or single treatment groups (FIG. 3C, quantified in FIG. 3D). Histologic (H&E) analysis of treated tumors revealed extensive tumor necrosis and micro-hemorrhage in tumors treated with NIR-PIT, while groups treated with PD-1 mAb demonstrated greater leukocyte infiltration (FIG. 3E). While primary tumor growth was inhibited following NIR-PIT or PD-1 mAb alone compared to control (FIG. 3F), combination treatment resulted in significant tumor control and complete rejection of established MC38-luc tumors in 9 of 13 (70%) mice. This response resulted in significantly prolonged survival in mice receiving combination treatment (FIG. 3G). While antibody treatment or anti-CD44-IR700 NIR-PIT increased survival time compared to control, none of the animals in the NIR-PIT group survived to 40 days and only 9% of those in the antibody only group (anti-CD44-IR700+anti-PD1 without NIR-PIT) survived to the end of the study (FIG. 3G). In contrast, 80% of the animals in the combination treatment group survived to the end of the study. Neither skin necrosis nor systemic toxicity was observed within any treatment group.

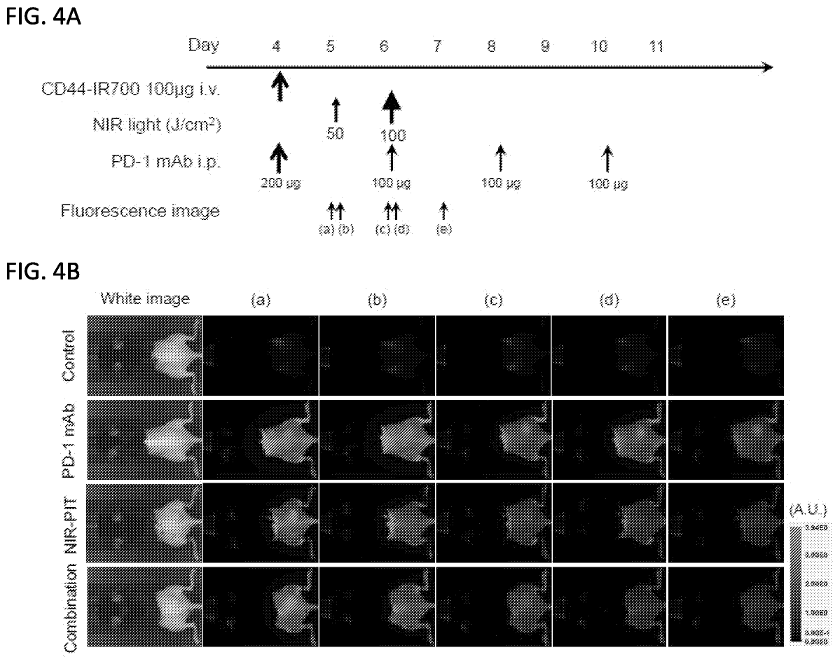

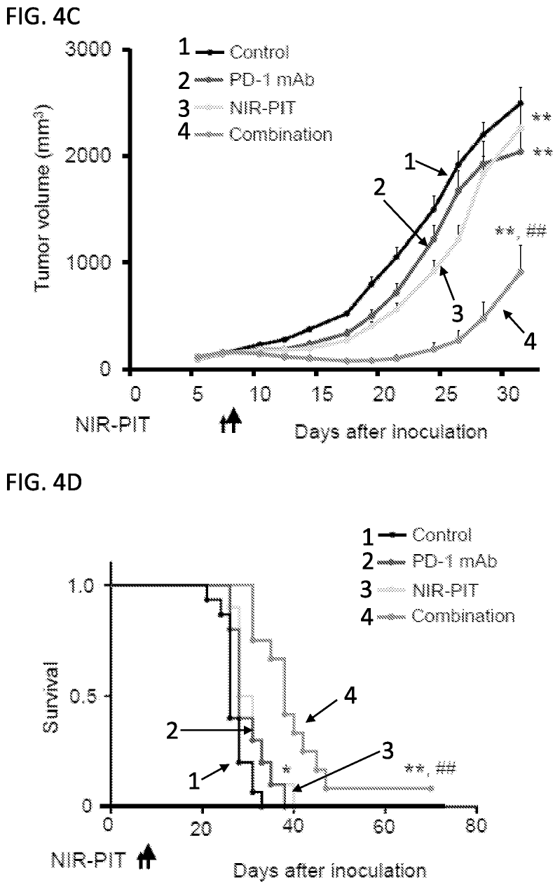

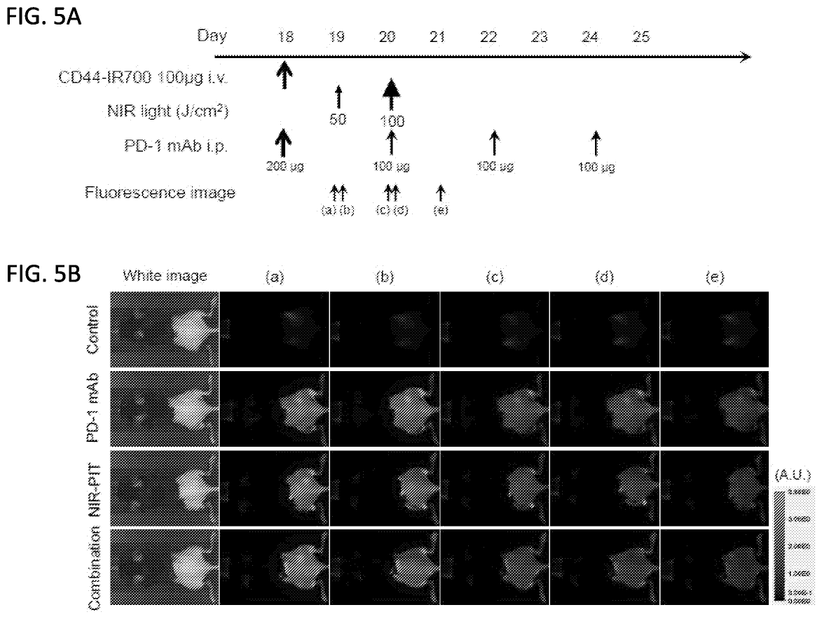

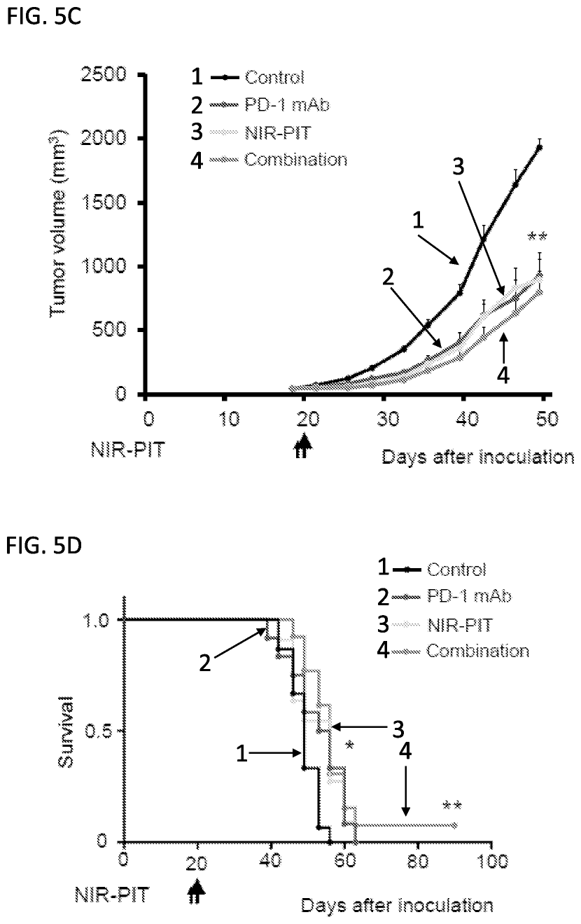

Similar approaches were taken in mice bearing established unilateral LLC or MOC1 tumors using similar treatment regimens and imaging protocols (FIGS. 4A, 5A). Similar to MC38-luc tumors, treatment of LLC or MOC1 tumors with NIR-PIT resulted in near-immediate loss of IR700 fluorescent signal (FIGS. 4B, 5B) indicating on-target effects. Treatment of LLC tumor-bearing mice with combination NIR-PIT and PD-1 mAb significantly enhanced primary tumor control (FIG. 4C) and survival (FIG. 4D) over control or either treatment alone, and resulted in rejection of 1 of 12 (8%) established tumors. Treatment of MOC1 tumor-bearing mice with combination NIR-PIT and PD-1 mAb induced rejection of 1 of 13 (8%) established tumors and resulted in statistically enhanced survival compared to control, but cumulative primary tumor growth following combination treatment was not enhanced over either treatment alone (FIGS. 5C, 5D).

Taken together, these results demonstrate CD44 on-target effects of NIR-PIT in MC38-luc, LLC and MOC1 tumor-bearing mice, with significant enhancement of primary tumor control and survival with the addition of PD-1 immune checkpoint blockade (ICB) in the MC38-luc and LLC models.

### Example 5

### Enhancement of Antigen-Specific Immunity Induction with NIR-PIT by PD-1 ICB

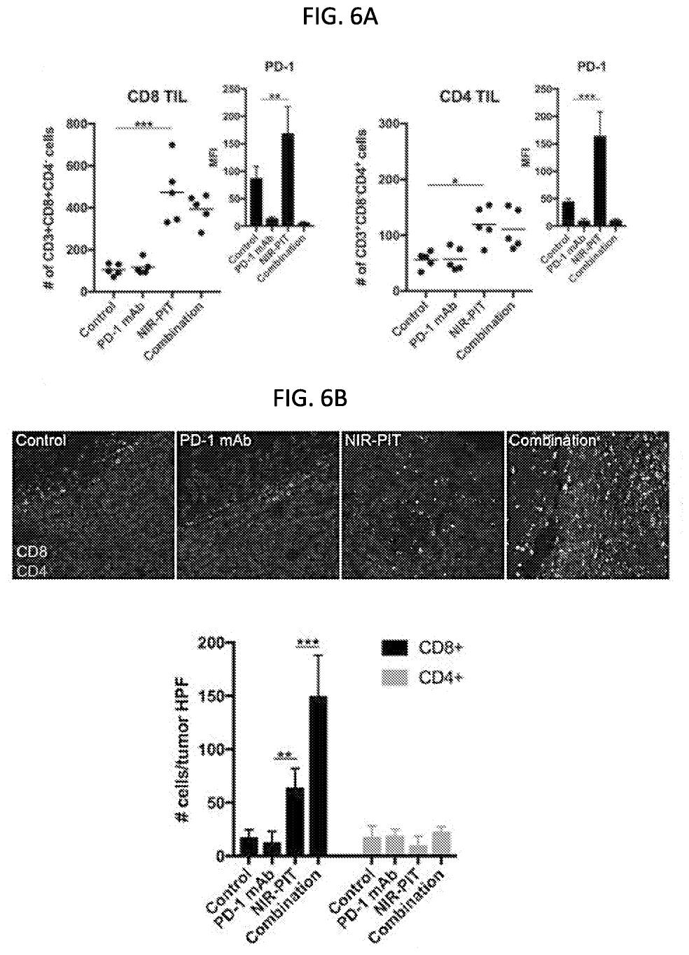

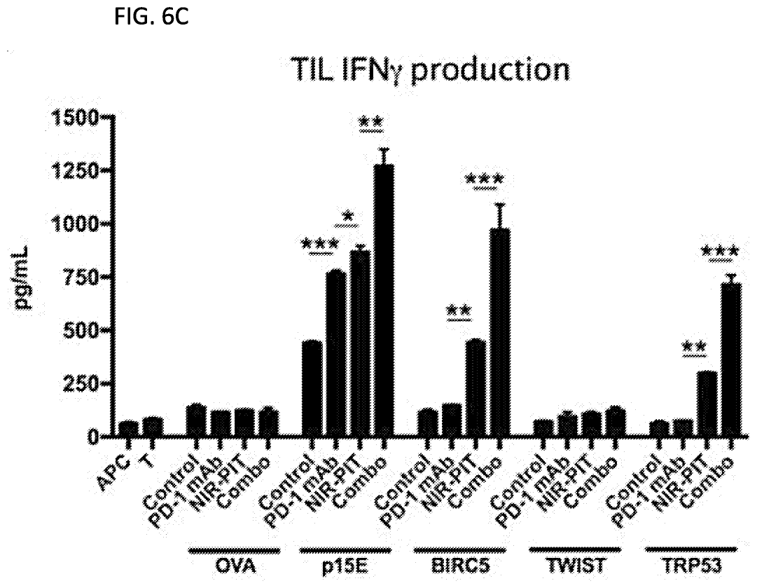

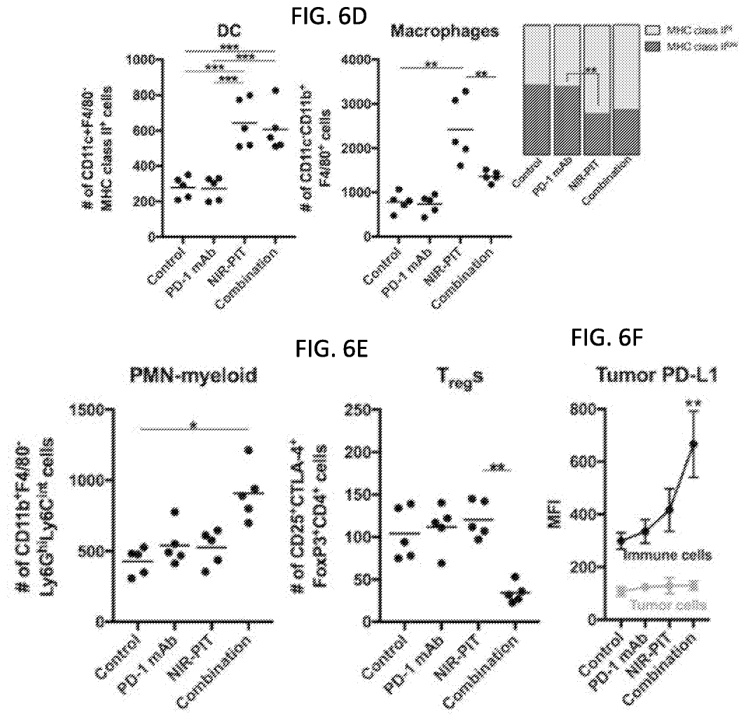

Following completion of treatment, some MC38-luc tumors were processed into single cell suspensions and assessed for infiltration of immune cells with flow cytometry. Tumors treated with NIR-PIT demonstrated significantly enhanced infiltration by CD8 and CD4 tumor infiltrating lymphocytes (TIL) (FIG. 6A) that expressed greater levels of PD-1. Mice treated with systemic PD-1 mAb demonstrated PD-1 target saturation as very low levels of PD-1 were detectible on the surface of TIL from these tumors by flow cytometry after staining with the same Ab clone (RMP1-14). This enhanced CD8 and CD4 TIL infiltration was verified by multiplex immunofluorescence (IF). In control or PD-1 mAb treated tumors, few CD8+ TIL nested along the tumor-stromal interface but did not infiltrate the tumor (FIG. 6B, left panels). Following NIR-PIT, more CD8+ TIL infiltrated throughout the tumor but many TIL were still arrested at the tumor-stromal interface. Infiltration into the tumor was significantly enhanced with the addition of PD-1 mAb (FIG. 6B, right panels). In additional experiments, TIL were extracted from control or treated MC38-luc tumors via IL-2, and assessed for antigen-specific IFNγ responses to multiple H-2K^{b} or H-2K^{d}-restricted TAA (FIG. 6C). TIL from control tumors demonstrated measurable responses to H-2K^{b}-restricted p15E₆₀₄₋₆₁₁ (KSPWFTTL) but lacked responses to other antigens. PD-1 mAb treatment enhanced the baseline p15E_{604_611} responses but did not induce responses against other antigens. NIR-PIT treatment induced de novo responses that were absent at baseline to H-2K^{b}-restricted Survivin/Birc5₅₇₋₆₄ (QCFFCFKEL) and H-2D^{b}-restricted Trp53₂₃₂₋₂₄₀ (KYMCNSSCM) and enhanced baseline responses to p15E₆₀₄₋₆₁₁. Treatment with PD-1 mAb enhanced these NIR-PIT induced or enhanced antigen-specific responses. NIR-PIT also enhanced tumor infiltration of MHC class II-positive dendritic cells (DCs) and F4/80+ macrophages polarized to express greater levels of MHC class II (FIG. 6D) Immunosuppressive neutrophilic-myeloid (PMN-myeloid) and regulatory CD4+T-lymphocytes (T_{reg}s) were variably altered by combination treatment (FIG. 6E). MC38-luc tumor cell specific PD-L1 expression was verified but did not change with treatment, while infiltrating immune cell PD-L1 was significantly greater than tumor cell expression, and increased with combination treatment (FIG. 6F).

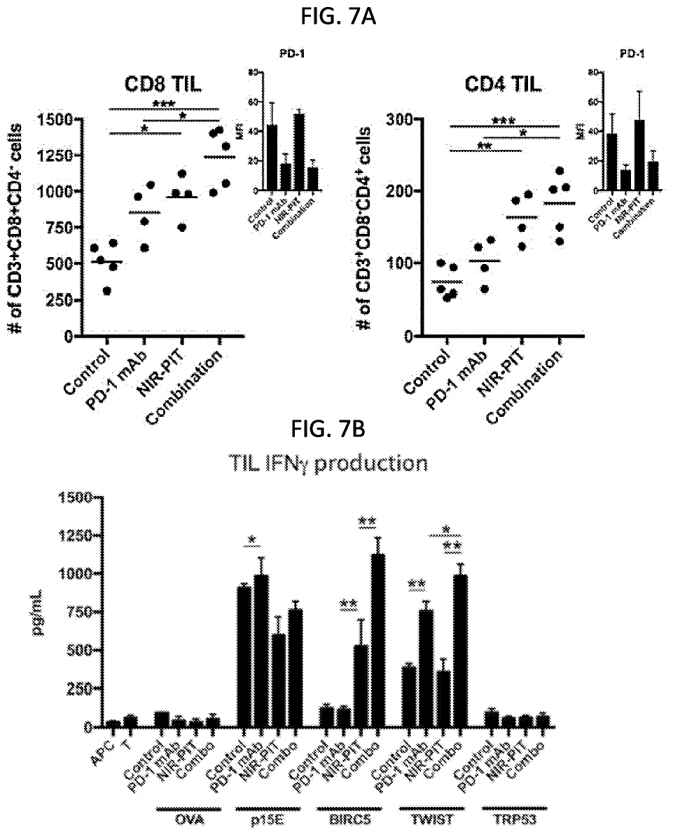

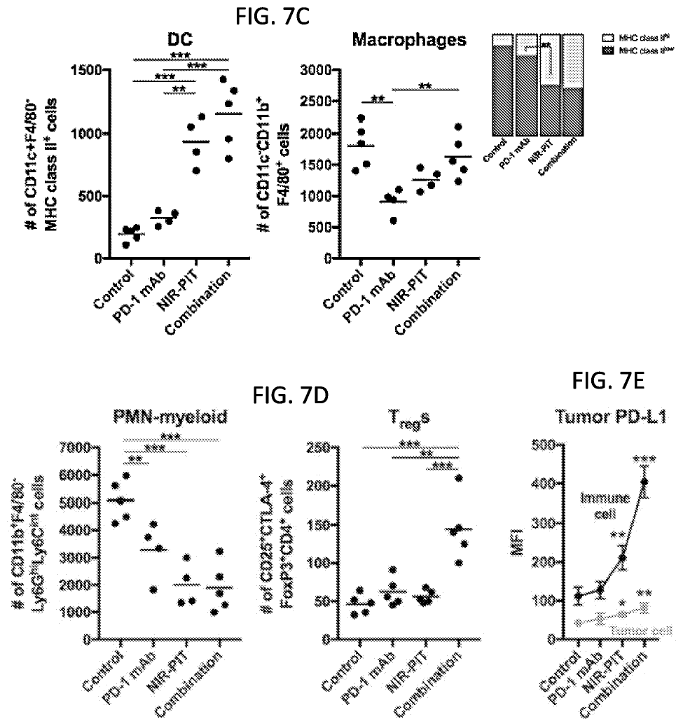

Similar immune correlative experiments were carried out in LLC and MOC1 tumors. LLC tumors treated with PD-1 mAb and NIR-PIT alone or in combination demonstrated enhanced TIL infiltration (FIG. 7A). Antigen-specific LLC TIL demonstrated measureable baseline responses to p15E₆₀₄₋₆₁₁ and H-2D^{b}-restricted Twist₁₂₅₋₁₃₃ (TQSLNEAFA). Similar to MC38-luc tumors, NIR-PIT treatment induced responses to Survivin/Birc5₅₇₋₆₄. Responses to Birc5 and Twist but not p15E were enhanced with PD-1 mAb treatment (FIG. 7B). NIR-PIT treatment of LLC tumors enhanced infiltration of MHC class II-positive DCs and MHC class II expression on macrophages (FIG. 7C). PMN-myeloid cells and L_{reg}s were variably altered following treatments FIG. 7D), and LLC tumor and immune cell-specific PD-L1 expression was enhanced with treatment (FIG. 7E).

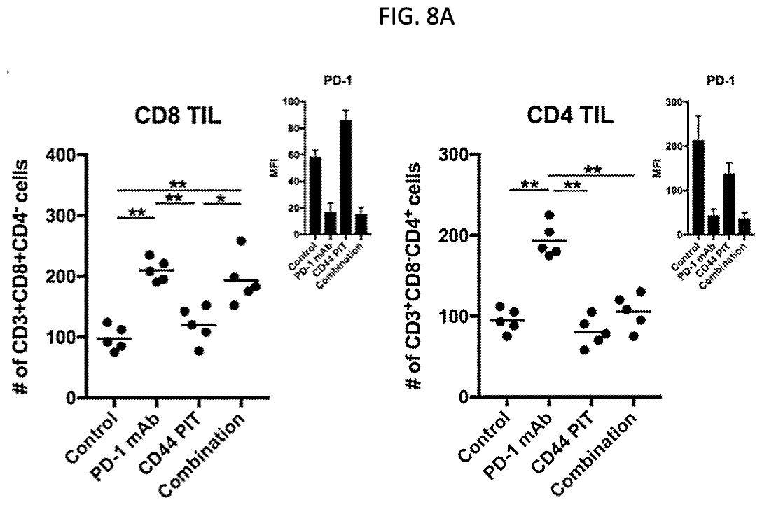

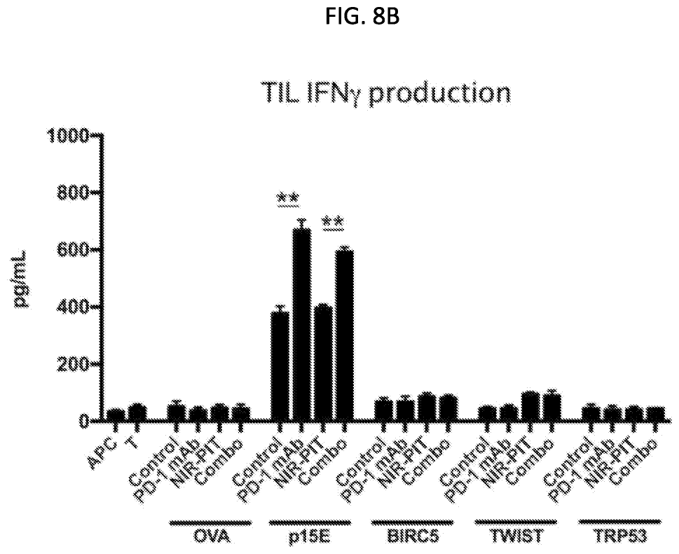

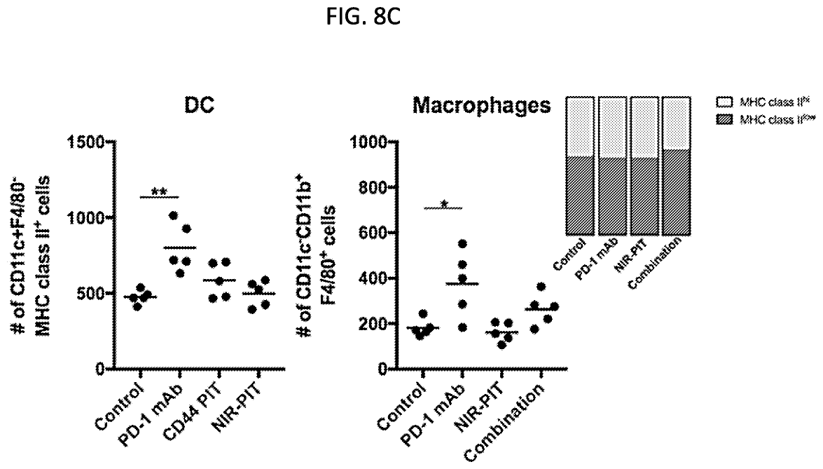

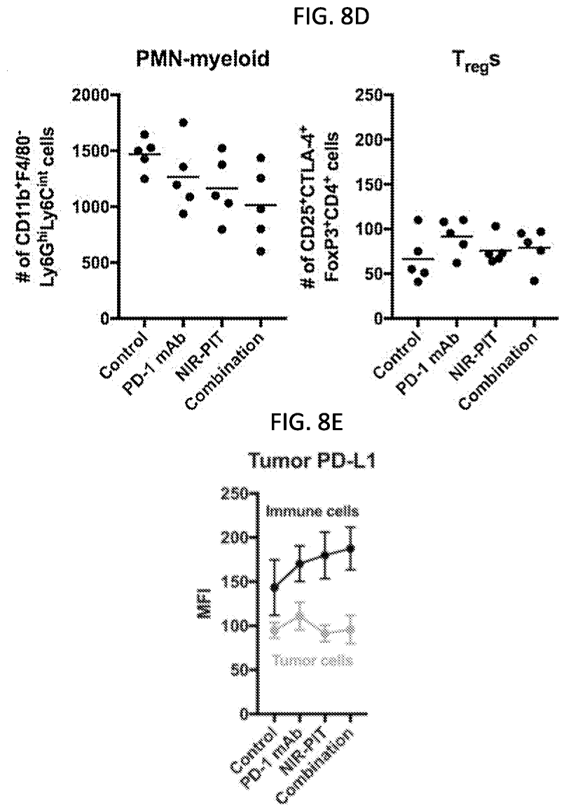

In contrast to MC38-luc or LLC tumors, MOC1 tumors treated with NIR-PIT demonstrated few immune correlative alterations. CD8 and CD4 TIL infiltration was modestly enhanced with PD-1 mAb but not NIR-PIT (FIG. 8A). Baseline TIL antigen-specific responses to p15E₆₀₄₋₆₁₁ were enhanced with systemic PD-1 mAb treatment, but responses to other shared tumor antigens were not induced with NIR-PIT treatment (FIG. 8B). MOC1 tumor infiltration of MHC class II+DCs and macrophages was modestly enhanced, indicating a lack of myeloid cell priming and activation in this model. No significant changes were observed in infiltration of PMN-myeloid cells or Tregs or MOC1 tumor or immune cell-specific PD-L1 expression (FIGS. 8C, 8D).

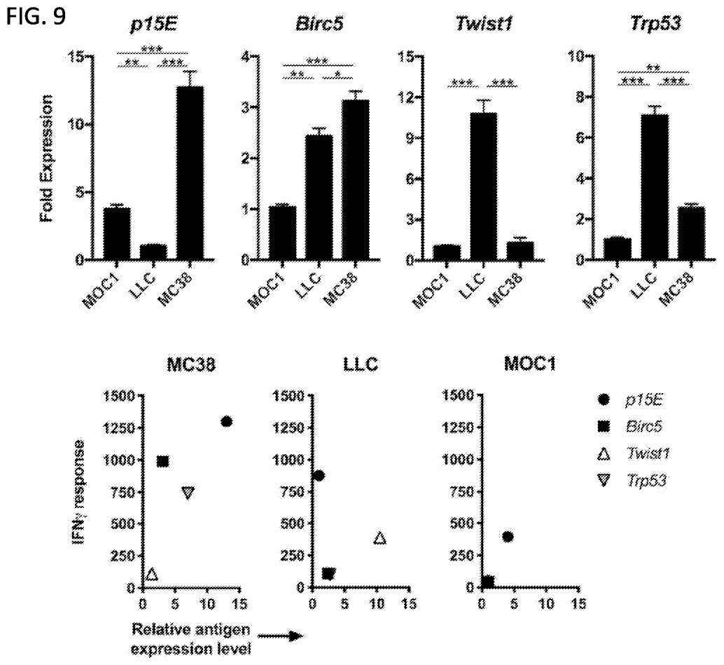

To investigate possible explanations for the lack of TIL responses against tumor associated antigens in MOC1, relative expression of each antigen was measured within MC38-luc, LLC and MOC1 cells. Using primers designed to flank the MHC class I-restricted epitope coding region, PCR results indicated low expression of Birc5, Twist1 and Trp53 gene transcripts in MOC1 relative to MC38-luc and LLC (FIG. 9). Greater antigen expression generally correlated with baseline TIL responses. Interestingly, higher relative Trp53 expression in MC38-luc cells and Twist1 expression in LLC cells correlated to enhanced TIL responses against the class I-restricted epitopes from these genes after combination NIR-PIT and PD-1 mAb treatment. Thus enhanced TIL responses after treatment may be dependent on baseline tumor antigen expression.

These results indicate that NIR-PIT can induce de novo, polyclonal antigen-specific TIL responses against MHC class I-restricted tumor antigens in MC38-luc and LLC tumor bearing mice, and that these responses can be enhanced with systemic PD-1 ICB.

### Example 6

### Combination NIR-PIT and PD-1 ICB Induced Abscopal Anti-Tumor Effect in Mice Bearing Bilateral MC38-Luc Tumors

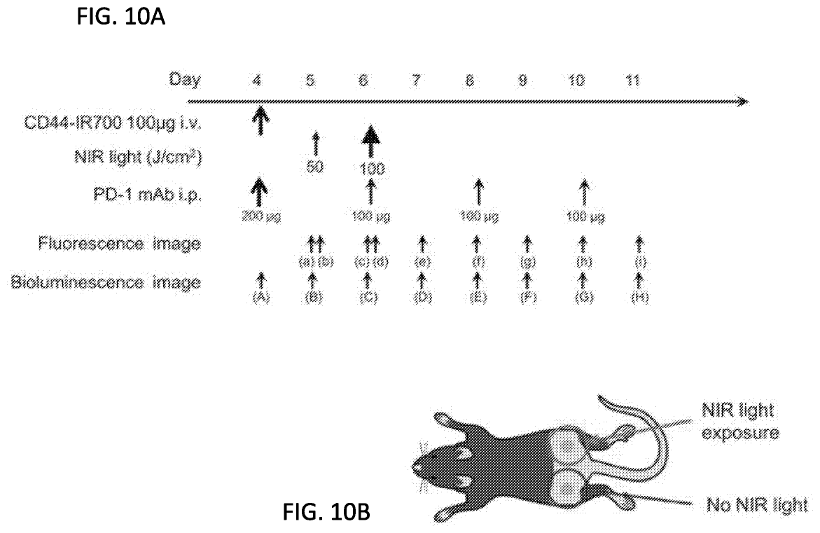

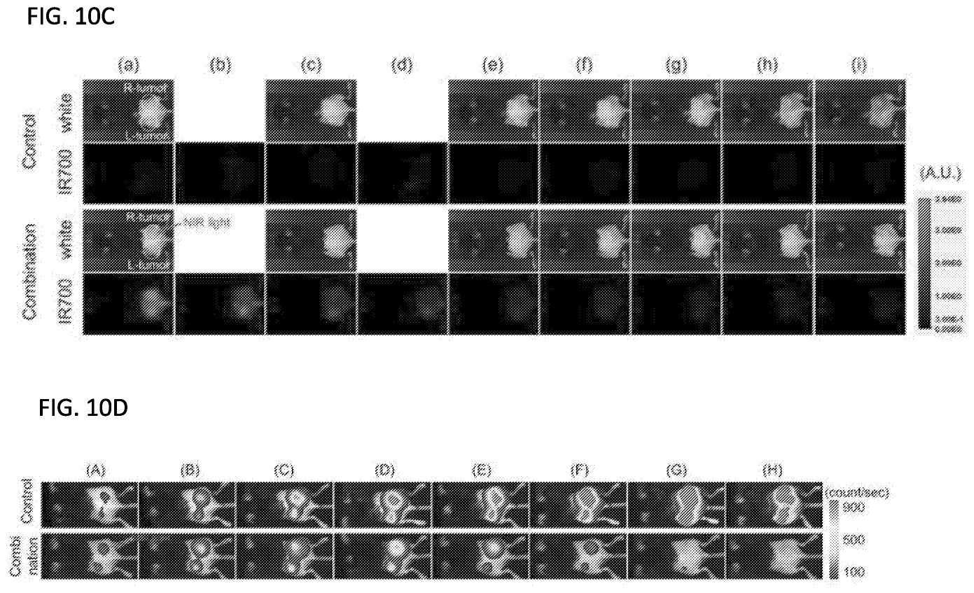

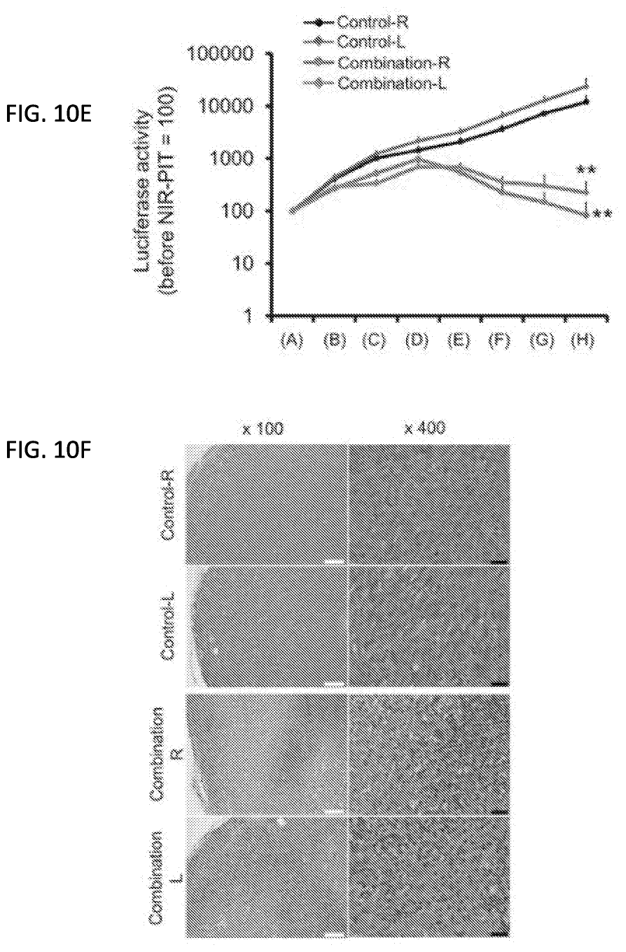

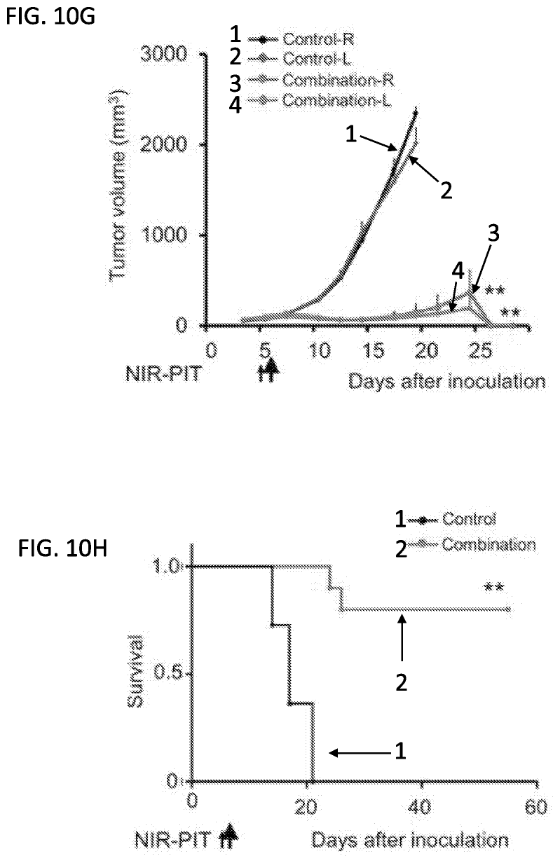

Given evidence of induction of tumor antigen-specific immunity following NIR-PIT in MC38-luc tumor bearing mice, whether local NIR-PIT combined with systemic PD-1 mAb could induce anti-tumor immunity in a separate, distant tumor not treated with NIR-PIT was determined. Treatment and imaging regimens (FIG. 10A) were similar for mice bearing bilateral MC38-luc tumors as described above, but only the right flank tumor was treated with NIR-PIT (FIG. 10B).

NIR-PIT induced near-immediate loss of IR700 fluorescent signal in the treated tumor, whereas loss of IR700 signal intensity in the untreated tumor was delayed for several days (FIG. 10C). Conversely, bioluminescence of both right (treated with NIR-PIT) and left (untreated) MC38-luc tumors decreased concurrently after combination treatment (FIG. 10D, quantified in FIG. 10E). Histologic analysis of both right and left tumors revealed similar patterns of necrosis and micro-hemorrhage and increase leukocyte infiltration (FIG. 10F). Combination treatment resulted in significant primary tumor control and complete tumor rejection of both right and left tumors in 8 of 10 mice (80%; FIG. 10G), leading to enhanced survival compared to untreated mice (FIG. 10H).

### Example 7

### Induction of Antigen-Specific Immunity in Distant Tumors not Treated with NIR-PIT

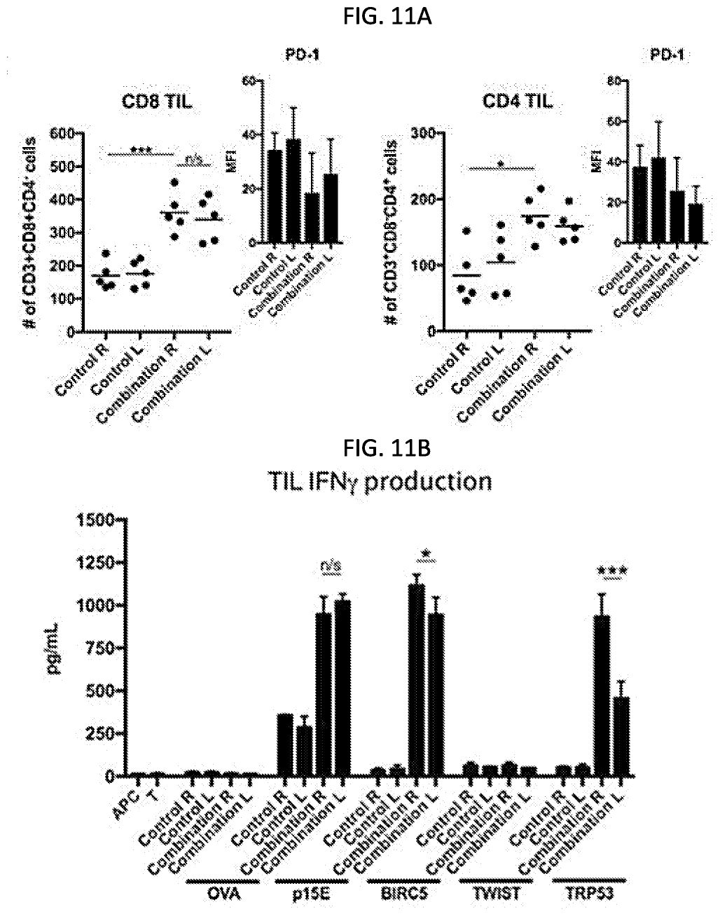

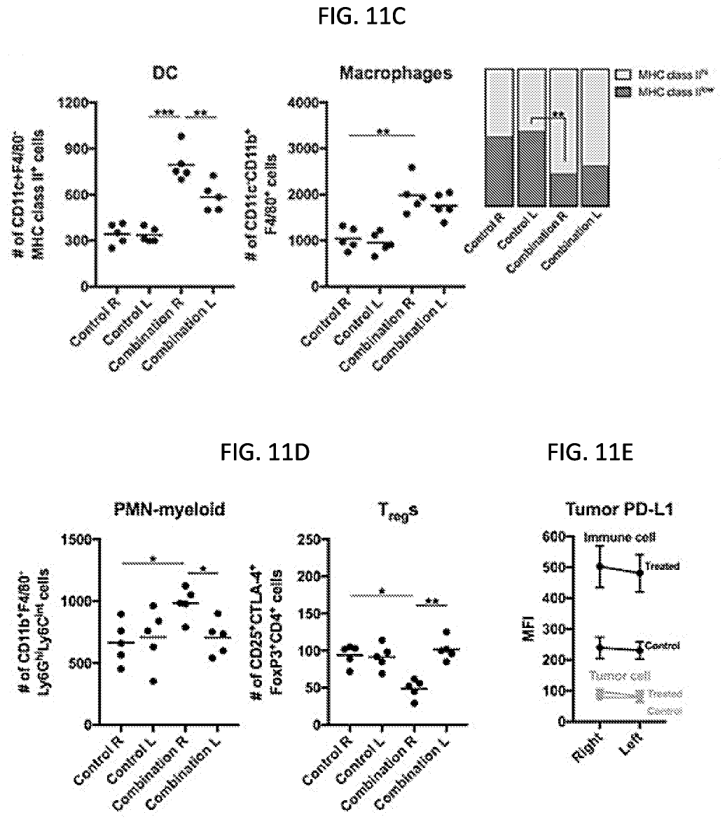

Flow cytometric analysis of single cell suspensions from both right (treated with NIR-PIT) and left (untreated) tumors revealed similar levels of enhanced CD8 and CD4 TIL accumulation (FIG. 11A). Assessment of antigen-specific reactivity demonstrated that TIL from both treated and untreated tumors reacted to the same MHC class I-restricted antigens (FIG. 11B), indicating the presence of systemic antigen-specific immunity. TIL responses were similar in magnitude to p15E₆₀₄₋₆₁₁ and Survivin/Birc5₅₇₋₆₄, but responses to Trp53₂₃₂₋₂₄₀ were diminished in tumors not treated with NIR-PIT compared to those treated. Increased MHC class II-positive DC and macrophages (FIG. 11C), increased PMN-myeloid cells and decreased T_{reg}s (FIG. 11D) were observed in treated but not untreated tumors, indicating these changes are a direct result of NIR-PIT and not a result of systemic anti-tumor immunity. MC38-luc infiltrating immune cell PD-L1 expression FIG. 11E) was enhanced in both right treated and left untreated tumors in mice receiving combination treatment, indicating that immune cell PD-L1 expression may be independent of NIR-PIT.

Thus, combination NIR-PIT and PD-1 ICB can lead to the development of systemic tumor antigen-specific immunity capable of eliminating an established untreated tumor, but enhanced innate immunity and alterations in immunosuppressive cell subsets appear to occur locally as a more direct effect of NIR-PIT.

### Example 8

### Combination NIR-PIT and PD-1 ICB Controls Multiple Distant Tumors in Mice with High Disease Burden

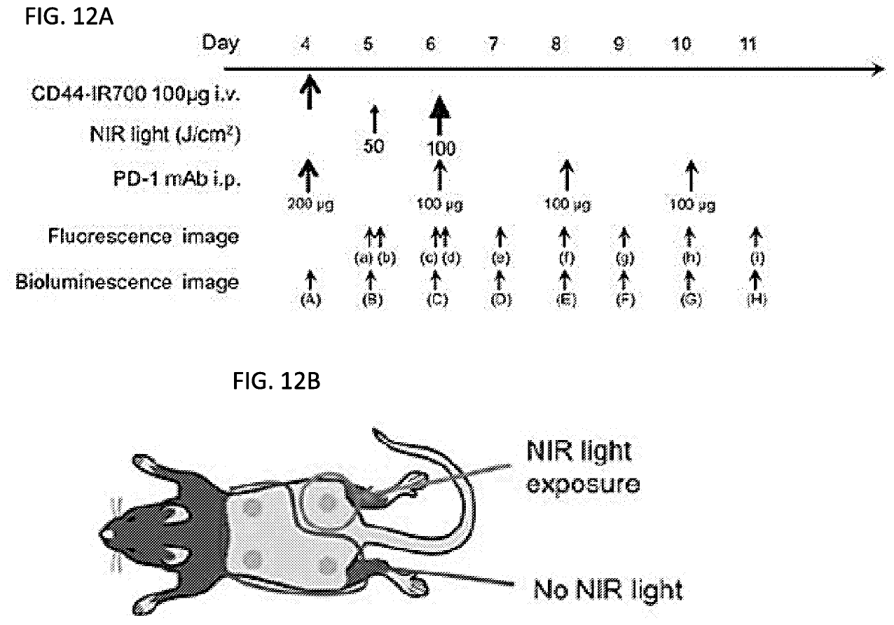

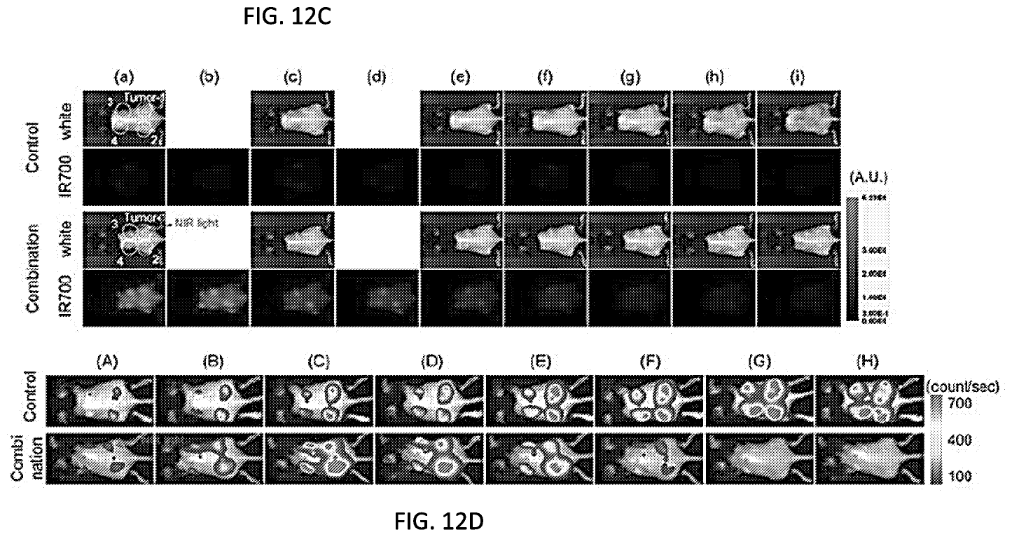

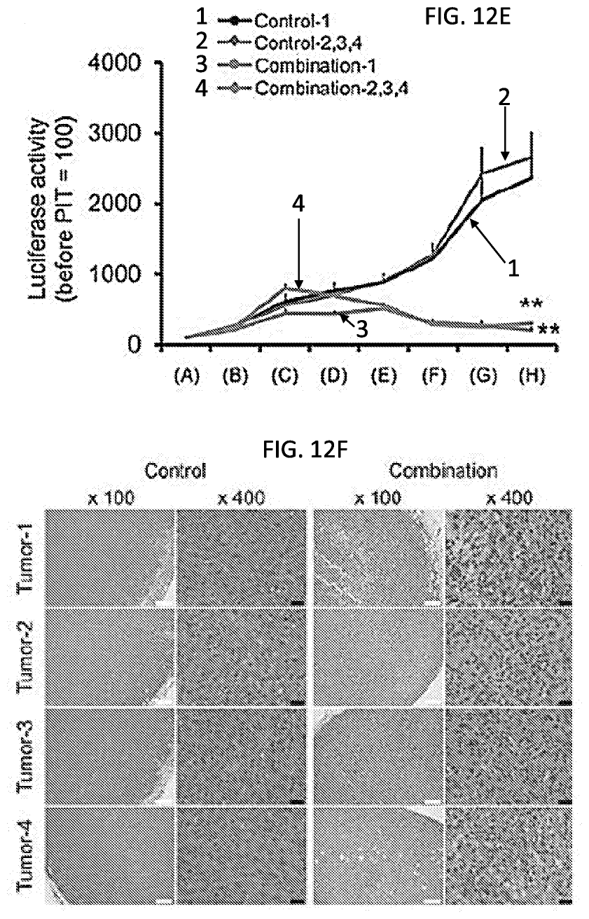

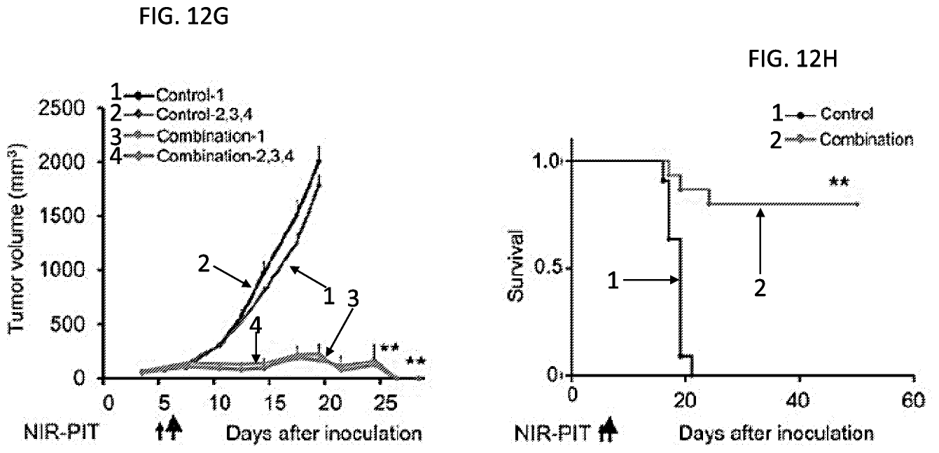

To demonstrate that treatment of a single MC38-luc tumor could lead to rejection of multiple established distant tumors within an individual mouse, the following methods were used. Similar treatments (FIG. 12A) were used to deliver NIR-PIT to one of four established MC38 tumors (FIG. 12B). NIR-PIT induced near-immediate loss of IR700 fluorescent signal in the single treated tumor, whereas resolution of IR700 signal intensity in the three untreated tumors was delayed for several days (FIG. 12C). Conversely, bioluminescence of both the single treated and three untreated MC38-luc tumors decreased concurrently after combination treatment (FIG. 12D, quantified in FIG. 12E). Histologic analysis revealed necrosis and increased leukocyte infiltration in all tumors from treated mice but not tumors from control mice (FIG. 12F). Systemic PD-1 mAb and treatment of a single MC38-luc tumor with NIR-PIT resulted in dramatic growth control multiple MC38-luc tumors. Twelve of 15 (80%) treated mice (FIG. 12G) completely rejected all four tumors, resulting in enhanced survival compared to control (FIG. 12H).

Thus, treatment of a single focus of tumor with local NIR-PIT plus systemic PD-1 ICB is sufficient to induce systemic immunity capable of eliminating multiple sites of distant disease not treated with NIR-PIT.

### Example 9

### Mice that Rejected Tumors after Combination NIR-PIT and PD-1 ICB Developed Immunologic Memory

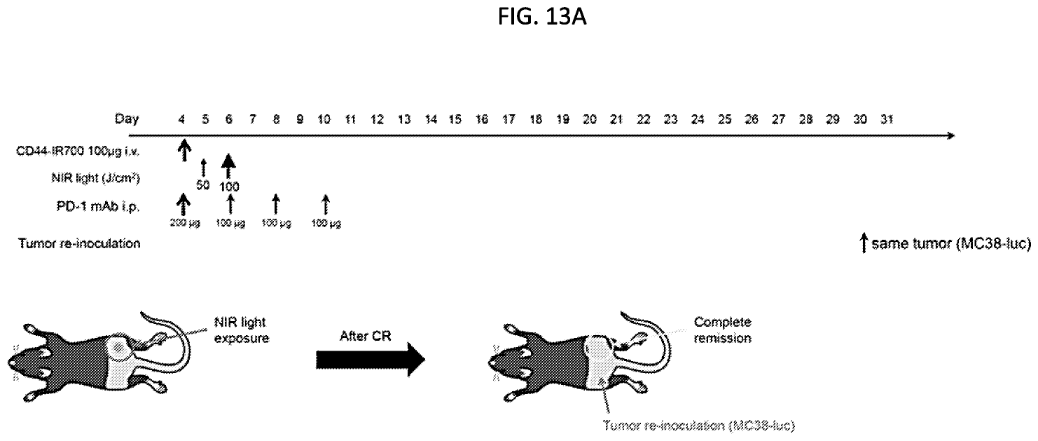

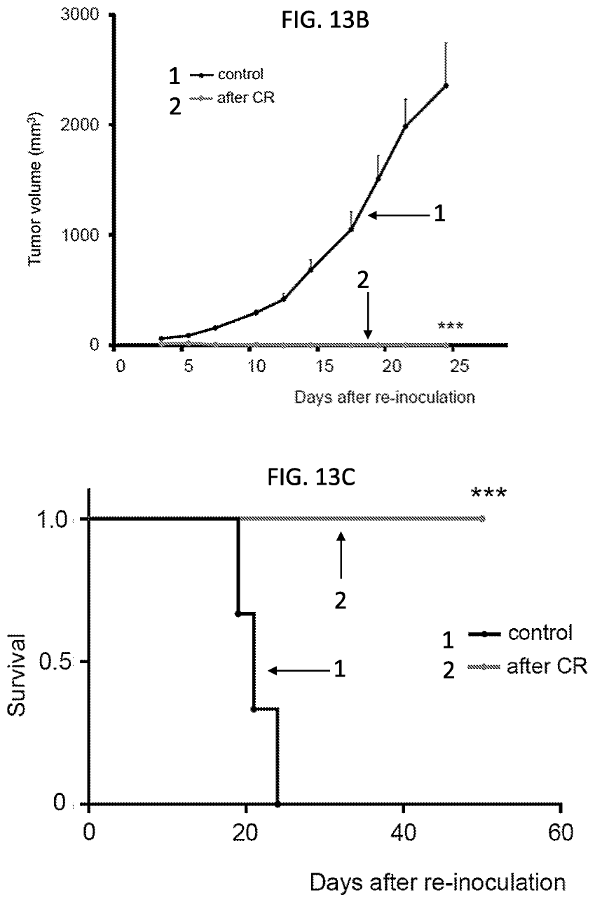

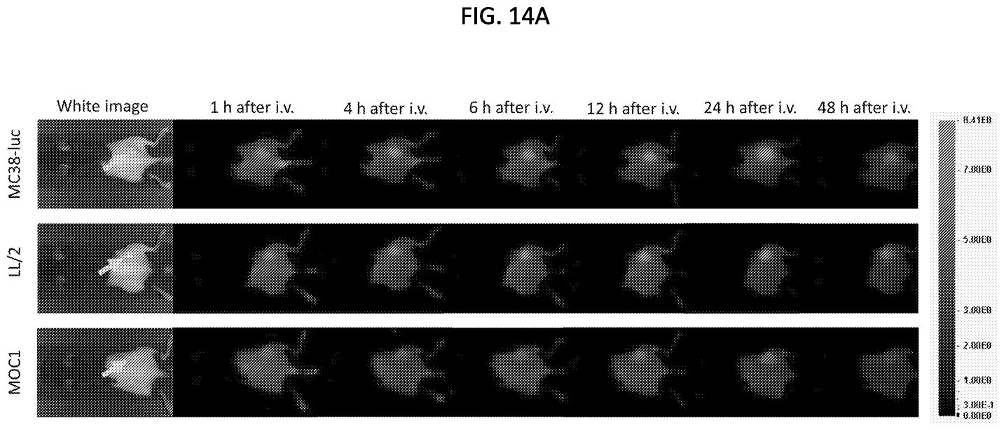

To assess for the presence of immunologic memory, mice were treated with NIR-PIT and PD-1 mAb as described above (FIG. 13A). Mice that demonstrated a complete response to combination treatment were challenged 30 days later with injection of MC38-luc cells in the contralateral flank (FIG. 13A). Whereas control mice readily were engrafted with MC38-luc tumors, mice that previously rejected established MC38-luc tumors resisted engraftment and did not grow tumors (FIG. 13C, survival in FIG. 13D), demonstrating the presence of immunologic memory.

As depicted in FIG. 18, the results in the examples above demonstrate that NIR-PIT induces CD44-specific tumor cell death, leading to the release of multiple tumor antigens. NIR-PIT also promotes a pro-inflammatory tumor microenvironment, resulting in cross priming of multiple antigens and the development of a polyclonal antigen-specific T-cell response. This effector response is limited by PD-1/PD-L1 expression and adaptive immune resistance, which is effectively reversed with the addition of PD-1 ICB.

### Example 10

### Materials and Methods

This example provides the materials and methods used to obtain the results described in Examples 11-14.

### Cell Culture

MC38-luc cells expressing CD44 and luciferase, LL/2 cells and MOC1 cells stably expressing CD44 antigen were cultured in RPMI1640 supplemented with 10% fetal bovine serum and 1% penicillin-streptomycin in tissue culture flasks in a humidified incubator at 37° C. in an atmosphere of 95% air and 5% carbon dioxide.

### Reagents

Water soluble, silica-phthalocyanine derivative, IRDye700DX NHS ester was from LI-COR Bioscience (Lincoln, Nebr., USA). An anti-mouse/human CD44 mAb (IM7) and anti-mouse CD25 mAb (PC-61.5.3) were from Bio X Cell. All other chemicals were of reagent grade.

### Synthesis of IR700-Conjugated Anti-CD25 mAb and Anti-CD44 mAb

Anti-CD25 mAb (1 mg, 6.7 nmol/L) and Anti-CD44 mAb (1 mg, 6.7 nmol/L) were respectively incubated with IR700 (65.1 μg, 33.3 nmol, 10 mmol/L in DMSO) and 0.1 mol/L Na₂HPO₄ (pH 8.5) at room temperature for 1 hour. The mixture was purified with a gel filtration column (Sephadex G 25 column, PD-10, GE Healthcare, Piscataway, N.J., USA). The protein concentration was determined with Coomassie Plus protein assay kit (Thermo Fisher Scientific Inc, Rockford, Ill., USA) by measurement of the absorption at 595 nm with spectroscopy (8453 Value System; Agilent Technologies, Santa Clara, Calif., USA). Herein, IR700-conjugated anti-CD25 mAb and anti-CD44 mAb are abbreviated as anti-CD25-mAb-IR700 and anti-CD44-mAb-IR700, respectively.

### Animal Model

Six- to eight-week-old female C57BL/6 mice (strain #000664) were purchased from the Jackson laboratory. The lower part of the body of the mice was shaved for irradiation and image analysis. Mice with tumors reaching approximately 150 mm·in in volume were used for the experiments. Tumor volumes were calculated from the greatest longitudinal diameter (length) and the greatest transverse diameter (width) using the following formula; tumor volume=length×width²×0.5, based on caliper measurements. Mice were monitored each day and tumor volumes were measured three times a week for MC38-luc and LL/2 tumors and twice a week for MOC1 tumors until the tumor volume reached 2,000 mm³, whereupon the mice were euthanized with inhalation of carbon dioxide gas.

### In Vivo Bioluminescence Imaging (BLI) and IR700 Fluorescence Imaging

To obtain bioluminescence images in MC38-luc tumor-bearing mice, D-luciferin (15 mg/mL, 150 μL) was intraperitoneally injected to mice. Luciferase activity was analyzed with a BLI system (Photon Imager; Biospace Lab, Paris, France) using relative light units (RLU). Regions of interest (ROI) were placed over the entire tumor. The counts per minute of RLU were calculated using M3 Vision Software (Biospace Lab) and converted to the percentage based on RLU before NIR-PIT (% RLU). BLI was performed before and after NIR-PIT on day 0 to day 7. In vivo IR700 fluorescence images were obtained with a Pearl Imager (LI-COR Biosciences) with a 700-nm fluorescence channel

### In Vivo Fluorescence Imaging Studies

MC38-luc cells (8 million), LL/2 cells (8 million) and MOC1 cells (4 million) were subcutaneously injected in the dorsum of the mice. Mice with tumors were studied after they reached volumes of approximately 150 mm³. Serial dorsal fluorescence images of IR700 were obtained with a Pearl Imager using a 700-nm fluorescence channel 1, 4, 6, 12, 24, and 48 hours after intravenous injection of 100 μg of anti-CD25-mAb-IR700 via the tail vein. Regions of interest (ROI) were placed on the tumor and the adjacent non-tumor region as background. The mean value of fluorescence intensity (MFI) was calculated for each ROI. Target-to-background ratio (TBR) was calculated from fluorescence intensities of tumors and fluorescence intensity of background by the following formula; (fluorescence intensity of tumor)-(fluorescence intensity of background)/(fluorescence intensity of background).

### NIR-PIT

MC38-luc cells (8 million), LL/2 cells (8 million) and MOC1 cells (4 million) were subcutaneously injected in the dorsum of mice. The mice with tumors which reached volumes of approximately 150 mm³ were selected and divided randomly into 4 experimental groups for the following treatments: (1) no treatment (control); (2) intravenous injection of 100 μg anti-CD25-mAb-IR700 followed by external NIR light irradiation at 100 J/cm·on day 0 (CD25-targeted NIR-PIT); (3) intravenous injection of 100 μg anti-CD44-mAb-IR700 followed by external NIR light irradiation at 100 J/cm^{·}on day 0 (CD44-targeted NIR-PIT); and (4) intravenous injection of 100 μg anti-CD25-mAb-IR700 and 100 μg anti-CD44-mAb-IR700 (combined NIR-PIT).

For the mice with MC38-luc tumor, LL/2 tumor, and MOC1 tumor in the NIR-PIT treated groups, intravenous injection of the APCs was performed 5, 5, and 28 days after tumor inoculation, respectively, followed by external NIR light irradiation at 100 J/cm·1 day after APC injection. NIR light was irradiated from above a targeted tumor in tumor-bearing mice using a red light emitting diode (LED), which emits light in the range of 670 to 710 nm wavelength (L690-66-60; Marubeni America Co.) at a power density of 50 mW/cm² as measured with an optical power meter (PM 100, Thorlabs). IR700 absorbs light at approximately 690 nm. IR700 fluorescence images were obtained before and after therapy.

### Statistical Analysis

Quantitative data were expressed as means±SEM. For multiple comparisons (≥3 groups), a one-way analysis of variance followed by the Tukey-Kramer test was used. The cumulative probability of survival was analyzed by the Kaplan-Meier survival curve analysis, and the results were compared with the Log-rank test. Statistical analysis was performed with JMP 13 software (SAS Institute, Cary, N.C.). A p value of less than 0.05 was considered significant.

### Example 11

### In Vivo Fluorescence Imaging after Administration of Anti-CD25-mAb-IR700

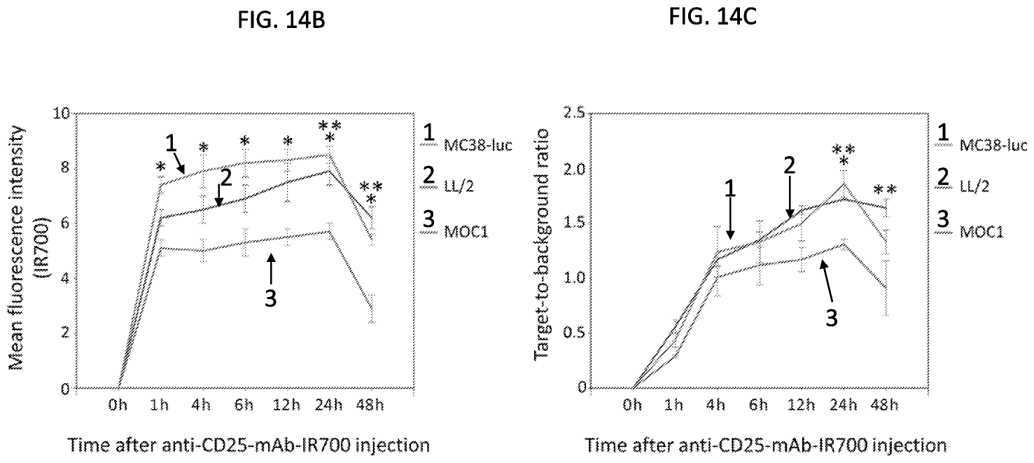

High fluorescence MFI was observed in MC38-luc, LL/2, and MOC1 1 hour after anti-CD25-mAb-IR700 (APC) injection, and fluorescence in all cell types gradually increased until 24 hours post injection (FIGS. 14A and 14B). The fluorescence 48 hours after APC injection decreased compared to the fluorescence at 24 hours. The TBR of anti-CD25-mAb-IR700 in all cell types also gradually increased until 24 hours followed by a decrease in TBR 48 hours after injection of the APC (FIG. 14C). The highest MFI and TBR were observed 24 hours after APC injection; MC38-luc and LL/2 tumors showed higher value in MFI and TBR than MOC1 tumors (FIGS. 14B and 14C).

These data demonstrate the rationale for delivery of therapeutic NIR light exposure 1 day after APC injection for both CD25- and/or CD44-targeted NIR-PIT in the examples below.

### Example 12

### Efficacy of Combined CD25- and CD44-Targeted NIR-PIT for MC38-Luc Tumor

FOXP3⁺CD25⁺CD4⁺ Treg cells are frequently found within tumors. In several types of cancers, decreased ratios of CD8⁺ T cells to FOXP3⁺CD25⁺CD4⁺ Treg cells in tumor-infiltrating lymphocytes (TILs) can be associated with poor prognosis. CD25-targeted NIR-PIT was used to deplete tumor-infiltrating Treg cells within the tumor without eliminating local effector cells or Treg cells in other organs, resulting in reversal of the permissive tumor microenvironment (TME) by removing immunosuppressive cells in the TME and subsequent tumor killing to enhance tumor directed NIR-PIT (achieved with the CD44-targeted NIR-PIT).

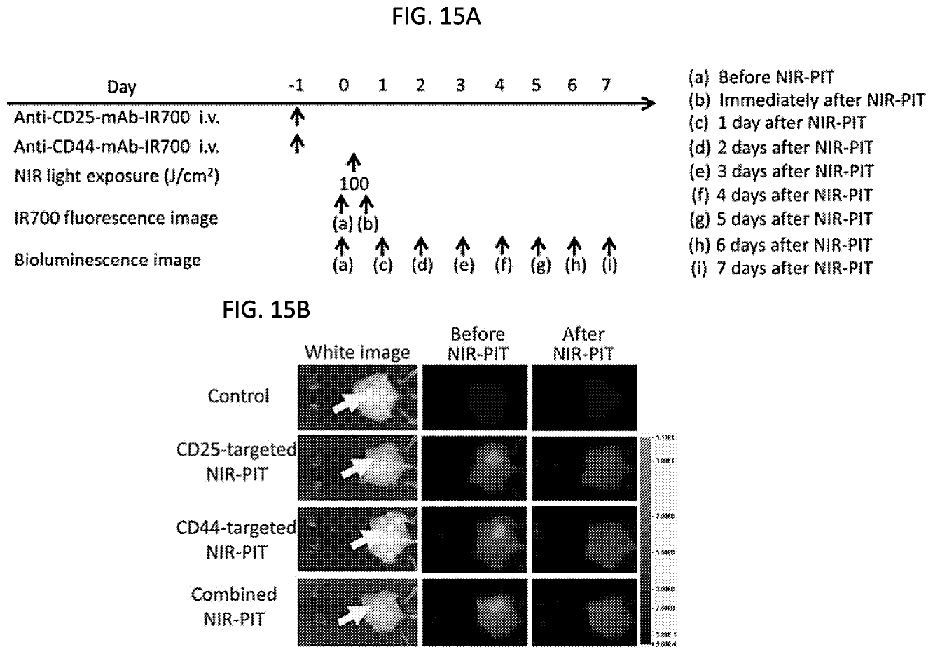

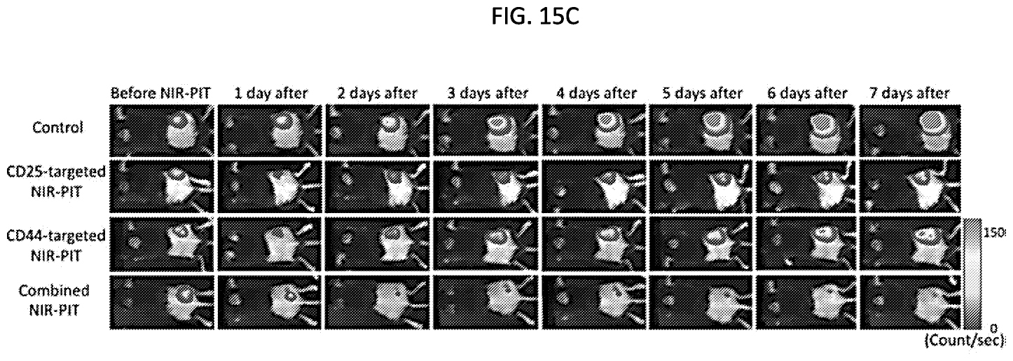

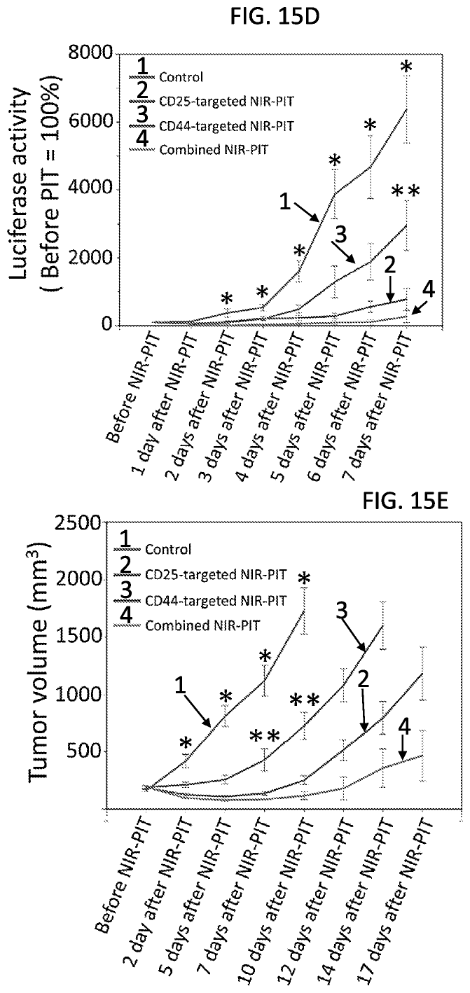

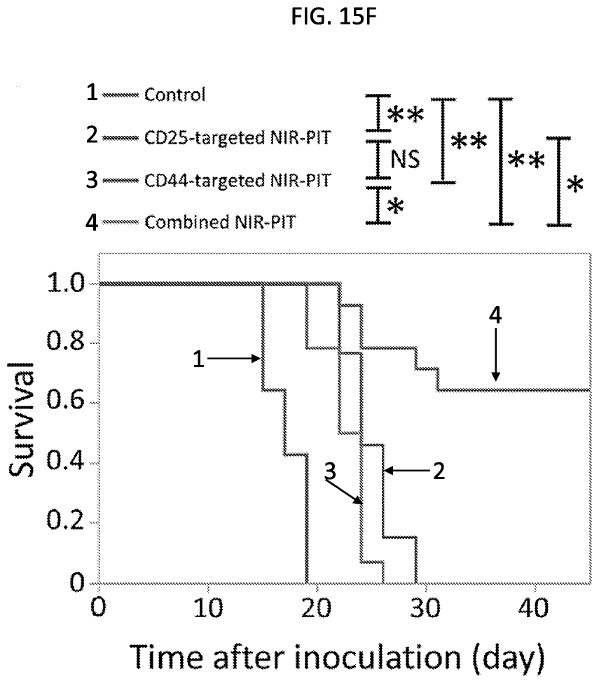

The NIR-PIT regimen and imaging protocol are depicted in FIG. 15A. One day after injection of anti-CD25- and/or anti-CD44-mAb-IR700, the tumors were exposed to 100 J/cm² of NIR light via LED light. IR700 tumor fluorescence signal decreased due to dispersion of fluorophore from dying cells and partial photo-bleaching in all cases (FIG. 15B).

To investigate tumor-killing efficacy after NIR-PIT, bioluminescence imaging (BLI) was performed before and after NIR-PIT up to day 7 (FIG. 15C). BLI was quantitatively evaluated as the percentage of RLU based on pre-treatment RLU (RLU Post/RLU Pre×100=% RLU). BLI is a highly sensitive tool for evaluating tumor cells after NIR-PIT and its intensity depends on the catalysis of luciferin by luciferase mediated by oxygen, Mg²⁺ and ATP.

In most mice in the NIR-PIT-treated groups, % relative light units (% RLU) greatly decreased shortly after NIR-PIT and then gradually increased (FIG. 15C). This pattern of % RLU change is likely due to a large amount of initial cell killing followed by slower regrowth of cells not originally killed. In contrast, in some mice undergoing CD25-targeted NIR-PIT and in the combined NIR-PIT groups, luciferase activity greatly decreased shortly after NIR-PIT and thereafter disappeared (FIG. 15C). This pattern of % RLU change is likely due to a large amount of initial cell killing followed by complete remission of treated tumors due to an enhanced immune response.

Post-treatment % RLU in all the NIR-PIT treated groups was significantly lower at all time points after NIR-PIT than in the control group (p<0.05, Tukey-Kramer test) (FIG. 15D). In addition, combined CD25- and CD44-targeted NIR-PIT showed significantly lower % RLU 7 days after NIR-PIT compared with CD44-targeted NIR-PIT alone (p<0.05, Tukey-Kramer test) (FIG. 15D). These data indicate that combined CD25- and CD44-targeted NIR-PIT can induce superior in vivo tumor-killing effects compared to either APC alone. Tumor volume in all the NIR-PIT treated groups was significantly inhibited 5, 7 and 10 days after NIR-PIT compared with that in the control group (p<0.05, Tukey-Kramer test) (FIG. 15E) but the combined CD25- and CD44-targeted NIR-PIT showed significantly greater tumor reduction compared to CD44-targeted NIR-PIT alone at 7 and 10 days after NIR-PIT (p<0.05, Tukey-Kramer test) (FIG. 15E). No significant tumor inhibition was observed in the other groups.

These data indicate that combined CD25- and CD44-targeted NIR-PIT led to the slowest rate of tumor regrowth compared with other NIR light exposure groups. Combined CD25- and CD44-targeted NIR-PIT also was associated with significantly prolonged survival after NIR-PIT compared with CD25-targeted NIR-PIT alone (p<0.05, Log-rank test) and CD44-targeted NIR-PIT alone (p<0.01, Log-rank test) (FIG. 15F). Moreover, 8 of 14 mice in the combined NIR-PIT group achieved complete remission after a single round of NIR-PIT.

These results show that combined CD25- and CD44-targeted NIR-PIT enables superior in vivo therapeutic responses compared to the other two types of NIR-PIT for MC38-luc tumors.

### Example 13

### Efficacy of Combination with CD25- and CD44-Targeted NIR-PIT for LL/2 Tumor

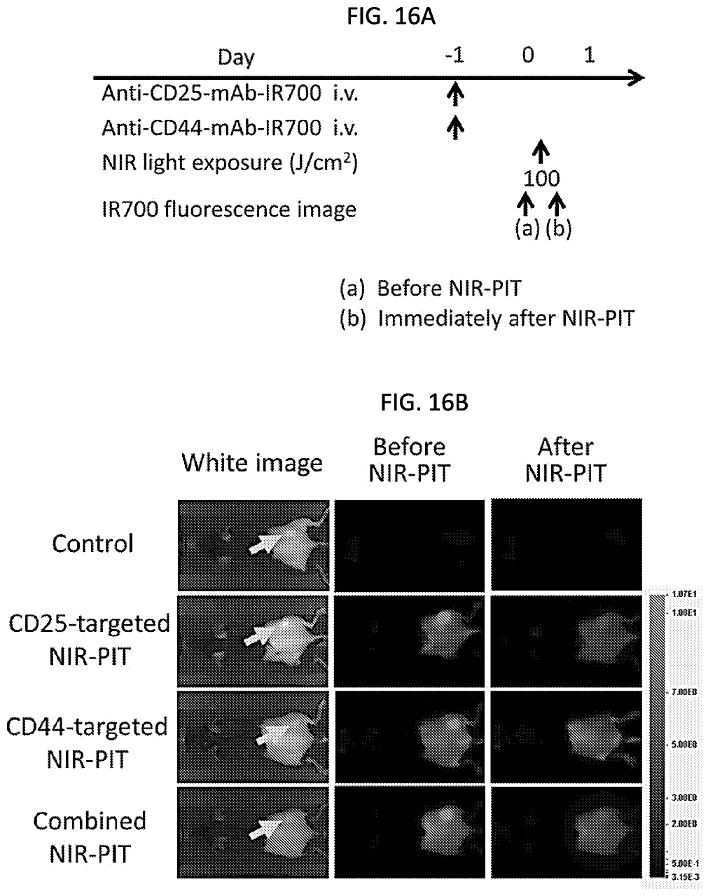

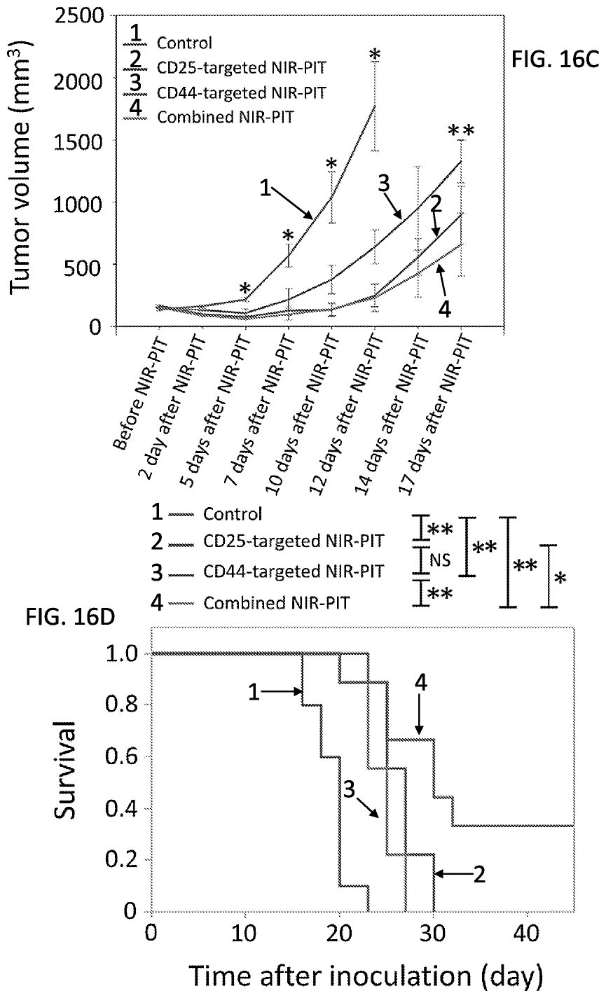

The NIR-PIT regimen and imaging protocol are depicted in FIG. 16A. One day after injection of anti-CD25- and/or anti-CD44-mAb-IR700, the tumors were exposed to 100 J/cm² of NIR light. IR700 tumor fluorescence signal decreased due to dispersion of fluorophore from dying cells and partial photo-bleaching (FIG. 16B). Tumor volume in all the NIR-PIT treated groups was significantly inhibited 5, 7, 10 and 12 days after NIR-PIT compared to that in the control group (p<0.05, Tukey-Kramer test) (FIG. 16C). Among the three NIR-PIT treated groups, combined CD25- and CD44-targeted NIR-PIT showed significantly greater tumor reduction compared to CD44-targeted NIR-PIT alone 17 days after NIR-PIT (p<0.05, Tukey-Kramer test) (FIG. 16C). In the long-term follow-up, combined CD25- and CD44-targeted NIR-PIT had significantly prolonged survival after NIR-PIT compared with CD25-targeted NIR-PIT alone or CD44-targeted NIR-PIT alone (p<0.05, Log-rank test) (FIG. 16D). In 3 of 9 mice in the combined NIR-PIT group complete remission of tumor was achieved after only a single round of NIR-PIT.

Thus, combined CD25- and CD44-targeted NIR-PIT was therapeutically superior to the other 2 types of NIR-PIT in LL/2 tumors.

### Example 14

### Efficacy of Combined CD25- and CD44-Targeted NIR-PIT for MOC1 Tumor

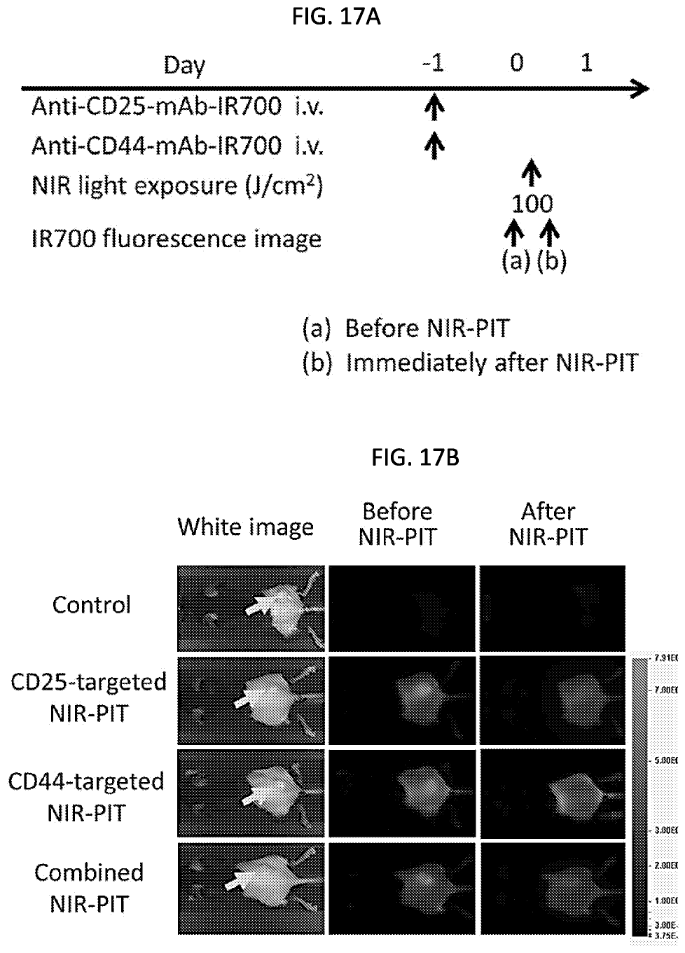

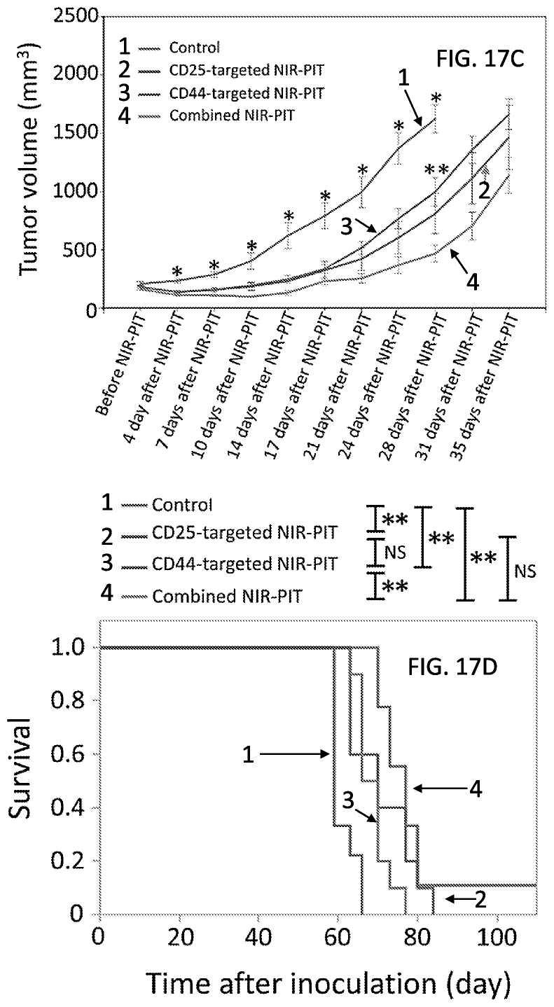

The NIR-PIT regimen and imaging protocol are depicted in FIG. 17A. One day after injection of anti-CD25- and/or anti-CD44-mAb-IR700, the tumors were exposed to 100 J/cm² of NIR light. IR700 tumor fluorescence signal decreased due to dispersion of fluorophore from dying cells and partial photo-bleaching. (FIG. 17B). Tumor volume in all the NIR-PIT treated groups was significantly inhibited at all time points after NIR-PIT compared to the control group (p<0.05, Tukey-Kramer test) (FIG. 17C). Combined CD25- and CD44-targeted NIR-PIT showed significantly greater tumor reduction 28 days after NIR-PIT compared to CD44-targeted NIR-PIT (p<0.05, Tukey-Kramer test).

In the long-term follow-up, combined CD25- and CD44-targeted NIR-PIT showed significantly prolonged survival compared to CD44-targeted NIR-PIT (p<0.05, Log-rank test) (FIG. 17D). On the other hand, there was no significant difference in tumor volume and survival between CD25-targeted NIR-PIT alone and CD44-targeted NIR-PIT alone, and between CD25-targeted NIR-PIT alone and the combined NIR-PIT (p>0.05, Tukey-Kramer test) (FIG. 17D). One of 9 mice in the combined NIR-PIT group achieved complete remission after a single round of NIR-PIT. Thus, combined CD25- and CD44-targeted NIR-PIT was superior therapeutically to the other two types of NIR-PIT in MOC1 tumors.

### Example 15

### Methods of Treating a Tumor

In one example, an antibody-IR700 molecule (such as anti-CD44-IR700) and an immunomodulator (such as an anti-PD1 antibody, anti-PD-L1 antibody, or anti-CD25-IR700) are administered to a subject with a tumor (day 1), such as a subject with cancer. The subject is then irradiated about 24 hours later with 50 J/cm² NIR light (day 2), and optionally with 100 J/cm² NIR light 24 hours after the first irradiation (day 3). The immunomodulator is also administered to the subject on days 3, 5, and 7, at the same or a different (for example, lower) dose.

The subject is monitored periodically for reduction of tumor size (such as tumor weight or volume), reduction in size or number of metastases, and/or survival (such as overall survival, progression-free survival, and/or disease-free survival).

In view of the many possible embodiments to which the principles of the disclosure may be applied, it should be recognized that illustrated embodiments are only examples of the disclosure and should not be considered a limitation on the scope of the invention. Rather, the scope of the invention is defined by the following claims. We therefore claim as our invention all that comes within the scope and spirit of these claims.

1. A method for treating a subject with cancer, comprising:
administering to the subject a therapeutically effective amount of one or more antibody-IR700 molecules, wherein the antibody specifically binds to a tumor-specific protein on the surface of a cancer cell;
irradiating the subject and/or irradiating cancer cells in the subject at a wavelength of 660 to 740 nm and at a dose of at least 1 J/cm²; and
administering to the subject a therapeutically effective amount of one or more immunomodulators,
wherein the one or more antibody-IR700 molecules and the one or more immunomodulators are administered sequentially or concurrently, and wherein the one or more antibody-IR700 molecules are administered prior to the irradiating step,
thereby treating the subject with cancer.

2. The method of claim 1, wherein the cancer cell is a cancer cell of the breast, liver, colon, ovary, prostate, pancreas, brain, cervix, kidney, bone, skin, head and neck, lung, or blood.

3. The method of claim 1, wherein the tumor-specific protein comprises CD44, HER1, HER2, CD20, CD25, CD33, CD52, CD44, CD133, Lewis Y, mesothelin, CEA, or prostate specific membrane antigen (PSMA).

4. The method of claim 1, wherein the subject and/or the cancer cells are irradiated at a wavelength of 680 nm.

5. The method of claim 1, wherein the cancer cells are in a subject's blood, and wherein irradiating the cancer cells comprises irradiating the blood by using a device worn by the subject, wherein the device comprises a near infrared (NIR) light emitting diode (LED).

6. The method of claim 1, wherein the method further comprises:
selecting a subject with a cancer that expresses the tumor-specific protein that specifically binds to the antibody-IR700 molecule.

7. The method of claim 1, wherein the method
reduces the volume or size of the cancer by at least 25% relative to the absence of treatment;
increases survival time of the subject relative to the absence of treatment, and/or
reduces the weight, volume, or size of a cancer and/or a metastasis not irradiated at a wavelength of 660 to 740 nm by at least 25%.

8.-10. (canceled)

11. The method of claim 1, wherein the one or more immunomodulators is an immune system activator and/or is an inhibitor of immuno-suppressor cells.

12. The method of claim 11, wherein the inhibitor of immuno-suppressor cells decreases activity of regulatory T (Treg) cells.

13. The method of claim 11, wherein the inhibitor of immuno-suppressor cells is daclizumab, denileukin difitox, cyclophosphamide, sorafenib, imatinib, an anti-PL-1 antibody, an anti-PD-L1 antibody, an anti-LAG-3 antibody, an anti-OX40 antibody, an anti-GITR antibody, or a combination of two or more thereof.

14. The method of claim 13, wherein
the anti-PL-1 antibody is nivolumab, pembrolizumab, pidilizumab, or cemiplimab; or
the anti-PL-L1 antibody is atezolizumab, avelumab, durvalumab, or BMS-936559.

15. (canceled)

16. The method of claim 12, wherein the decrease in Treg cell activity comprises killing Treg cells.

17. The method of claim 16, wherein killing Treg cells comprises administering to the subject a therapeutically amount of one or more antibody-IR700 molecules, wherein the antibody specifically binds to the suppressor cell surface protein,
wherein the antibody does not include a functional Fc region; and/or
wherein the suppressor cell surface protein is one or more of cluster of differentiation 4 (CD4), C-X-C chemokine receptor type 4 (CXCR4), C-C chemokine receptor type 4 (CCR4), cytotoxic T-lymphocyte-associated protein 4 (CTLA4), glucocorticoid induced TNF receptor (GITR), OX40, folate receptor 4 (FR4), CD25, CD16, CD56, CD8, CD122, CD23, CD163, CD206, CD11b, Gr-1, CD14, interleukin 4 receptor alpha chain (IL-4Ra), interleukin-1 receptor alpha (IL-1Ra), interleukin-1 decoy receptor, fibroblast activation protein (FAP), CD103, CXCR2, CD33, and CD66b; and/or
irradiating the suppressor cell at a wavelength of 660 to 740 nm and at a dose of at least 4 J/cm²; thereby killing the suppressor cell.

18. (canceled)

19. The method of claim 17, wherein the antibody that specifically binds to CD25
is daclizumab or basiliximab; and/or
does not include a functional Fc region.

20. (canceled)

21. The method of claim 11, wherein the immune system activator comprises one or more interleukins.

22. (canceled)

23. The method of claim 1, wherein irradiating the subject and/or irradiating cancer cells in the subject comprises
irradiating the subject and/or irradiating the cancer cells about 0 to 48 hours, such as about 24 hours, after administering the one or more antibody-IR700 molecules that specifically bind to the cancer cell surface protein; and/or
two or more doses of irradiation at a wavelength of 660 to 740 nm and at a dose of at least 1 J/cm².

24. (canceled)

25. The method of claim 23, wherein the two or more doses of irradiation are administered within about 12 to 36 hours of one another.

26. The method of claim 1, wherein the subject is administered two or more doses of the one or more immunomodulators.

27. (canceled)

28. The method of claim 1, further comprising:
detecting the cancer cell with fluorescence lifetime imaging about 0 to 48 hours after the irradiating step.

29. A method for treating a subject with cancer, comprising:
administering to the subject a therapeutically effective amount of an anti-CD44-IR700 molecule;
irradiating the subject and/or irradiating cancer cells in the subject at a wavelength of 660 to 740 nm and at a dose of at least 1 J/cm²; and
administering to the subject a therapeutically effective amount of an anti-PD-1 antibody, an anti-PD-L1 antibody, or both,
wherein the anti-CD44-IR700 molecule and the anti-PD-1 antibody, an anti-PD-L1 antibody, or both, are administered sequentially or concurrently, and wherein the anti-CD44-IR700 molecule is administered prior to the irradiating step,
thereby treating the subject with cancer.

30. A method for treating a subject with cancer, comprising:
administering to the subject a therapeutically effective amount of an anti-CD44-IR700 molecule;
administering to the subject a therapeutically effective amount of an anti-CD25-IR700 molecule; and
irradiating the subject and/or irradiating cancer cells in the subject at a wavelength of 660 to 740 nm and at a dose of at least 1 J/cm²;
wherein the anti-CD44-IR700 molecule and the anti-CD25-IR700 molecule are administered sequentially or concurrently, and wherein the anti-CD44-IR700 molecule and the CD25-IR700 molecule are administered prior to the irradiating step, thereby treating the subject with cancer.

31. A method of producing memory T cells, comprising:
administering to a subject a therapeutically effective amount of one or more antibody-IR700 molecules, wherein the antibody specifically binds to a tumor-specific protein on the surface of a cancer cell;
irradiating the subject and/or irradiating cells in the subject at a wavelength of 660 to 740 nm and at a dose of at least 1 J/cm²; and
administering to the subject a therapeutically effective amount of one or more immunomodulators,
wherein the one or more antibody-IR700 molecules and the one or more immunomodulators are administered sequentially or concurrently, and wherein the one or more antibody-IR700 molecule is administered prior to the irradiating step,
thereby producing memory T cells.

32. A method of killing a cancer cell in a subject's blood, comprising:
administering to the subject a therapeutically effective amount of one or more antibody-IR700 molecules, wherein the antibody specifically binds to a tumor-specific protein on the surface of a cancer cell;
irradiating the cancer cell with a NIR LED at a wavelength of 660 to 740 nm at a dose of at least 20 J/cm2, wherein the NIR LED is present in a wearable device worn by the subject; and
administering to the subject an effective amount of one or more immunomodulators,
wherein the one or more antibody-IR700 molecules and the one or more immunomodulators are administered sequentially or concurrently, and wherein the one or more antibody-IR700 molecule is administered prior to the irradiating step,
thereby killing the cancer cell.
